(19) United States
(12) Patent Application Publication
(10) Pub. No.: US 2026/0022144 A1
(43) Pub. Date: Jan. 22, 2026

(54) ANALGESIC POLYPEPTIDE

(71) Applicant: Shanghai Puyou Biomedical Co., Ltd., Shanghai (CN)

(72) Inventors: Yinghao Zhang, Shanghai (CN); Guirui Yan, Shanghai (CN); Yao Wang, Shanghai (CN)

(21) Appl. No.: 19/080,460

(22) Filed: Mar. 14, 2025

Related U.S. Application Data
(63) Continuation of application No. PCT/CN2023/118814, filed on Sep. 14, 2023.

(30) Foreign Application Priority Data
Sep. 15, 2022 (WO) PCT/CN2022/119040
Mar. 14, 2023 (WO) PCT/CN2023/081352

(51) Int. Cl. C07K 14/00; A61K 9/00; A61K 38/00; A61P 25/04

(52) U.S. Cl. CPC C07K 14/001; A61K 9/0019; A61K 9/0048; A61P 25/04; A61K 38/00

(57) ABSTRACT

The present disclosure provides analgesic peptides. Also provided is a formulation comprising any one of the analgesic peptides and a pharmaceutically acceptable excipient. Also provided is a method for treating or preventing pain in a subject in need thereof, comprising administering to the subject an effective amount of any one of the analgesic peptides.

### CROSS-REFERENCE

This application is a continuation of International Application No. PCT/CN2023/118814 filed on Sep. 14, 2023, which claims priority to International Application No. PCT/CN2022/119040 filed on Sep. 15, 2022, and to International Application No. PCT/CN2023/081352 filed on Mar. 14, 2023, the contents of which are herein incorporated by reference in their entirety.

### SEQUENCE LISTING

The instant application contains a Sequence Listing which has been submitted electronically in XML format and is hereby incorporated by reference in its entirety. Said XML copy, created on Mar. 14, 2025, is named 64982-711.301_SL.xml and is 102,409 bytes in size.

### BACKGROUND

According to the definition as provided by the international association for the study of pain (IASP) in 2020, pain is known as an unpleasant sensory and emotional experience associated with, or resembling that associated with, actual or potential tissue damage. Pain is always a personal experience that is influenced to varying degrees by biological, psychological, and social factors. Although pain usually serves an adaptive role, it may have adverse effects on function and social and psychological well-being. Pain may be a symptom caused by a disease, or alternatively, regarded as a separate disease.

The classification of pain has always been very complex. Generally, pain may be divided into 3 main categories: acute pain (for a duration of less than 3 months), chronic pain (for a duration of more than 3 to 6 months) and transitional pain (for a duration of 3 to 6 months) according to the course of the disease. The chronic pain may be further divided into nociceptive pain (caused by persistent inflammation and tissue damage), neuropathic pain (caused by nerve damage) and mixed pain (for a pain condition having the characteristics of both nociceptive pain and neuropathic pain).

The current clinical measures for pain management include drug therapy, minimally invasive intervention, neuromodulation, surgery and other treatment methods. Among them, drug therapy is regarded as the most basic and commonly used treatment method for pain. To date, there is still an unmet need in the medical community for drugs that effectively manage pain.

### SUMMARY

The present disclosure provides analgesic peptides. Also provided is a formulation comprising any one of the analgesic peptides and a pharmaceutically acceptable excipient. Also provided is a method for treating or preventing pain in a subject in need thereof, comprising administering to the subject an effective amount of any one of the analgesic peptides.

In one aspect, provided is an artificial polypeptide of formula (VI):

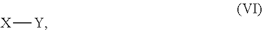

wherein X is a moiety comprising a mutant of the sequence SEQ ID NO: 1, characterized in that the mutant has at least 1, 2, 3, 4, 5, 6, 7, 8, or 9 D in SEQ ID NO: 1 mutated to S, and/or at least 1, 2, 3, 4, 5, or 6 E in SEQ ID NO: 1 mutated to T; and

Y is a moiety comprising a mutant of the sequence SEQ ID NO: 2, characterized in that the mutant has at least 1, 2, 3, 4, or 5 C in SEQ ID NO: 2 mutated to A.

In some embodiments. X comprises a sequence having at least 70% identity with SEQ ID NO: 1 and/or X comprises a sequence having at most 95% identity with SEQ ID NO: 1. In some embodiments. X comprises a sequence having at least 70% identity with SEQ ID NO: 1. In some embodiments. X comprises a sequence having at most 95% identity with SEQ ID NO: 1. In some embodiments, a total number of R, K, T. A. N. Q. D. E. S, and G in X is more than 30 and/or a total number of W, Y, F, M, L, I, and V in X is no more than 20. In some embodiments, a total number of R, K, T, A, N, Q, D, E, S, and G in X is more than 30. In some embodiments, a total number of W, Y, F, M, L, I, and V in X is no more than 20. In some embodiments. X comprises a sequence having at least 80% identity with any one selected from SEQ ID NOs: 80-83. X comprises a sequence that is any one selected from SEQ ID NOs: 80-83, or X is a sequence that is any one selected from SEQ ID NOs: 80-83. In some embodiments. X comprises a sequence having at least 80% identity with any one selected from SEQ ID NOs: 80-83. In some embodiments. X comprises a sequence that is any one selected from SEQ ID NOs: 80-83. In some embodiments. X is a sequence that is any one selected from SEQ ID NOs: 80-83.

In some embodiments. Y comprises a sequence having at least 80% identity with SEQ ID NO: 2 and/or Y comprises a sequence having at most 95% identity with SEQ ID NO: 2. In some embodiments. Y comprises a sequence having at least 80% identity with SEQ ID NO: 2. In some embodiments. Y comprises a sequence having at most 95% identity with SEQ ID NO: 2. In some embodiments, at least 50% amino acids of Y are selected from I, V, L, F, C, M, and A. In some embodiments, a total number of R, K, T, A, N, Q, D, E, S, and G in Y is more than 10 and/or a total number of W, Y, F, M, L, I, and V in Y is no more than 20. In some embodiments, a total number of R, K, T, A, N, Q, D, E, S, and G in Y is more than 10. In some embodiments, a total number of W, Y, F, M, L, I, and V in Y is no more than 20. In some embodiments. Y comprises a sequence having at least 80% identity with SEQ ID NO: 3. Y comprises a sequence of SEQ ID NO: 3, or Y is a sequence of SEQ ID NO: 3. In some embodiments. Y comprises a sequence having at least 80% identity with SEQ ID NO: 3. In some embodiments. Y comprises a sequence of SEQ ID NO: 3. In some embodiments. Y is a sequence of SEQ ID NO: 3.

In some embodiments, the artificial polypeptide comprises a sequence having at least 80% identity with any one selected from SEQ ID NOs: 90-93, the artificial polypeptide comprises a sequence that is any one selected from SEQ ID NOs: 90-93, or the artificial polypeptide is a sequence that is any one selected from SEQ ID NOs: 90-93. In some embodiments, the artificial polypeptide comprises a sequence having at least 80% identity with any one selected from SEQ ID NOs: 90-93. In some embodiments, the artificial polypeptide comprises a sequence that is any one selected from SEQ ID NOs: 90-93. In some embodiments, the artificial polypeptide is a sequence that is any one selected from SEQ ID NOs: 90-93.

In another aspect, provided is an artificial polypeptide of formula (VII):

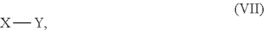

wherein X is a moiety comprising a mutant of the sequence SEQ ID NO: 1, characterized in that the mutant has at least 1, 2, 3, 4, or 5 amino acid insertions relative to SEQ ID NO: 1, and
Y is a moiety comprising 10 to 30 amino acids, wherein Y comprises a sequence having at least 15 continuous AAs of SEQ ID NO: 2.

In some embodiments, the amino acid insertions are located at the N-terminal of X and/or the inserted amino acid is M. In some embodiments, the amino acid insertions are located at the N-terminal of X. In some embodiments, the inserted amino acid is M.

In some embodiments, at least 50% amino acids of X are selected from R, K, N, D, Q, E, and H and/or at most 60% amino acids of X are selected from R, K, N, D, Q, E, and H. In some embodiments, at least 50% amino acids of X are selected from R, K, N, D, Q, E, and H. In some embodiments, at most 60% amino acids of X are selected from R, K, N, D, Q, E, and H. In some embodiments, a total number of E or D in X is at least 8 and/or a total number of E or D in X is at most 15. In some embodiments, a total number of E or D in X is at least 8. In some embodiments, a total number of E or D in X is at most 15. In some embodiments, a total number of R, K, T, A, N, Q, D, E, S, and G in X is more than 30 and/or a total number of W, Y, F, M, L, I, and V in X is no more than 20. In some embodiments, a total number of R, K, T, A, N, Q, D, E, S, and G in X is more than 30. In some embodiments, a total number of W, Y, F, M, L, I, and V in X is no more than 20. In some embodiments. X comprises a sequence having at least 80% identity with SEQ ID NO: 96. X comprises a sequence of SEQ ID NO: 96, or X is a sequence of SEQ ID NO: 96. In some embodiments. X comprises a sequence having at least 80% identity with SEQ ID NO: 96. In some embodiments. X comprises a sequence of SEQ ID NO: 96. In some embodiments. X is a sequence of SEQ ID NO: 96.

In some embodiments. Y is a moiety comprising a mutant of the sequence SEQ ID NO: 2, characterized in that the mutant has at least 1, 2, 3, 4, 5, 6, 7, 8, 9, 10, 11, 12, 13, 14, 15, 16, 17, 18, 19, or 20 amino acid truncations relative to SEQ ID NO: 2 and optionally the at least 1, 2, 3, 4, 5, 6, 7, 8, 9, 10, 11, 12, 13, 14, 15, 16, 17, 18, 19, or 20 amino acid truncations are located at the C-terminal of Y. In some embodiments. Y is a moiety comprising a mutant of the sequence SEQ ID NO: 2, characterized in that the mutant has at least 1, 2, 3, 4, 5, 6, 7, 8, 9, 10, 11, 12, 13, 14, 15, 16, 17, 18, 19, or 20 amino acid truncations relative to SEQ ID NO: 2. In some embodiments, the at least 1, 2, 3, 4, 5, 6, 7, 8, 9, 10, 11, 12, 13, 14, 15, 16, 17, 18, 19, or 20 amino acid truncations are located at the C-terminal of Y. In some embodiments. Y comprises 15 to 25 amino acids. Y comprises 18 to 25 amino acids, or Y comprises 20 to 25 amino acids. In some embodiments. Y comprises 15 to 25 amino acids. In some embodiments. Y comprises 18 to 25 amino acids. In some embodiments. Y comprises 20 to 25 amino acids. In some embodiments. 40-65% amino acids of Y are selected from I, V, L, F, C. M, and A. In some embodiments, a total number of R, K, T, A, N, Q, D, E, S, and G in Y is no more than 10 and/or a total number of W, Y, F, M, L, I, and V in Y is no more than 15. In some embodiments, a total number of R, K, T, A, N, Q, D, E, S, and G in Y is no more than 10. In some embodiments, a total number of W, Y, F, M, L, I, and V in Y is no more than 15. In some embodiments. Y comprises a sequence having at least 80% identity with any one selected from SEQ ID NOs: 97-103. Y comprises a sequence that is any one selected from SEQ ID NOs: 97-103, or Y is a sequence that is any one selected from SEQ ID NOs: 97-103. In some embodiments. Y comprises a sequence having at least 80% identity with any one selected from SEQ ID NOs: 97-103. In some embodiments. Y comprises a sequence that is any one selected from SEQ ID NOs: 97-103. In some embodiments. Y is a sequence that is any one selected from SEQ ID NOs: 97-103.

In some embodiments, the artificial polypeptide comprises a sequence having at least 80% identity with any one selected from SEQ ID NOs: 104-110, the artificial polypeptide comprises a sequence that is any one selected from SEQ ID NOs: 104-110, or the artificial polypeptide is a sequence that is any one selected from SEQ ID NOs: 104-110. In some embodiments, the artificial polypeptide comprises a sequence having at least 80% identity with any one selected from SEQ ID NOs: 104-110. In some embodiments, the artificial polypeptide comprises a sequence that is any one selected from SEQ ID NOs: 104-110. In some embodiments, the artificial polypeptide is a sequence that is any one selected from SEQ ID NOs: 104-110.

In another aspect, provided is mutant of artificial polypeptide of formula (I).

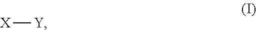

wherein X is a moiety comprising a sequence having at least 80% identity with SEQ ID NO: 1.
Y is a moiety comprising 10 to 50 amino acids, at least 50% of which are selected from I, V, L, F, C, M, and A.
Y comprises a total number of Cysteine (C) of less than 5.
characterized in that the mutant has at least 1, 2, 3, 4, 5, 6, 7, 8, or 9 D in X mutated to S, and/or at least 1, 2. 3, 4, 5, or 6 E in X mutated to T.

In some embodiments, at least 20% amino acids of X are selected from R, K, N, D, Q, E, and H, at least 30% amino acids of X are selected from R, K, N, D, Q, E, and H, at most 90% amino acids of X are selected from R, K, N, D, Q, E, and H, and/or at most 80% amino acids of X are selected from R, K, N, D, Q, E, and H. In some embodiments, at least 20% amino acids of X are selected from R, K, N, D, Q, E, and H. In some embodiments, at least 30% amino acids of X are selected from R, K, N, D, Q, E, and H. In some embodiments, at most 90% amino acids of X are selected from R, K, N, D, Q, E, and H. In some embodiments, at most 80% amino acids of X are selected from R, K, N, D, Q, E, and H. In some embodiments. X comprises a sequence having at least 70% identity with SEQ ID NO: 1.

In some embodiments. Y comprises a total number of Cysteine (C) of less than 4, or Y comprises a total number of Cysteine (C) of less than 3. In some embodiments. Y comprises a total number of Cysteine (C) of less than 4. In some embodiments. Y comprises a total number of Cysteine (C) of less than 3. In some embodiments, a total number of hydrophobic amino acids in Y is more than 8, the total number of hydrophobic amino acids in Y is more than 12, the total number of hydrophobic amino acids in Y is more than 15, and/or a total number of hydrophilic amino acids in Y is no more than 5. In some embodiments, a total number of hydrophobic amino acids in Y is more than 8. In some embodiments, the total number of hydrophobic amino acids in Y is more than 12. In some embodiments, the total number of hydrophobic amino acids in Y is more than 15. In some embodiments, a total number of hydrophilic amino acids in Y is no more than 5. In some embodiments. Y comprises a sequence having at least 90% identity with any one selected from SEQ ID NOs: 3-15, 58-61 and 84-85, or Y comprises a sequence having at least 90% identity with any one selected from SEQ ID NOs: 3-18, 58-61 and 84-85. In some embodiments. Y comprises a sequence having at least 90% identity with any one selected from SEQ ID NOs: 3-15, 58-61 and 84-85. In some embodiments. Y comprises a sequence having at least 90% identity with any one selected from SEQ ID NOs: 3-18. 58-61 and 84-85.

In another aspect, provided is mutant of artificial polypeptide of formula (II).

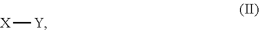

wherein X is a moiety comprising a sequence having at least 80% identity with SEQ ID NO: 1.
Y is a moiety comprising 10 to 50 amino acids, at least 50% of which are selected from I, V, L, F, C, M, and A.
Y comprises a sequence having at most 90% identity with SEQ ID NO: 2.
characterized in that the mutant has at least 1, 2, 3, 4, 5, 6, 7, 8, or 9 D in X mutated to S, and/or at least 1, 2, 3, 4, 5, or 6 E in X mutated to T.

In some embodiments, at least 20% amino acids of X are selected from R, K, N, D, Q, E, and H, at least 30% amino acids of X are selected from R, K, N, D, Q, E, and H, at most 90% amino acids of X are selected from R, K, N, D, Q, E, and H, and/or at most 80% amino acids of X are selected from R, K, N, D, Q, E, and H. In some embodiments, at least 20% amino acids of X are selected from R, K, N, D, Q, E, and H. In some embodiments, at least 30% amino acids of X are selected from R, K, N, D, Q, E, and H. In some embodiments, at most 90% amino acids of X are selected from R, K, N, D, Q, E, and H. In some embodiments, at most 80% amino acids of X are selected from R, K, N, D, Q, E, and H. In some embodiments. X comprises a sequence having at least 70% identity with SEQ ID NO: 1.

In some embodiments. Y comprises a sequence having at most 80% identity with SEQ ID NO: 2, or Y comprises a sequence having at most 70% identity with SEQ ID NO: 2. In some embodiments. Y comprises a sequence having at most 80% identity with SEQ ID NO: 2. In some embodiments. Y comprises a sequence having at most 70% identity with SEQ ID NO: 2. In some embodiments, a total number of hydrophobic amino acids in Y is more than 8, the total number of hydrophobic amino acids in Y is more than 12, the total number of hydrophobic amino acids in Y is more than 15, and/or a total number of hydrophilic amino acids in Y is no more than 5. In some embodiments, a total number of hydrophobic amino acids in Y is more than 8. In some embodiments, the total number of hydrophobic amino acids in Y is more than 12. In some embodiments, the total number of hydrophobic amino acids in Y is more than 15. In some embodiments, a total number of hydrophilic amino acids in Y is no more than 5. In some embodiments. Y comprises a sequence having at least 90% identity with any one selected from SEQ ID NOs: 16-18, or Y comprises a sequence having at least 95% identity with any one selected from SEQ ID NOs: 16-18. In some embodiments. Y comprises a sequence having at least 90% identity with any one selected from SEQ ID NOs: 16-18. In some embodiments. Y comprises a sequence having at least 95% identity with any one selected from SEQ ID NOs: 16-18.

In another aspect, provided is a mutant of artificial polypeptide of formula (III).

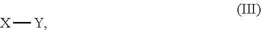

wherein X is a moiety comprising 40 to 65 amino acids, at least 50% of which are selected from H, R, K, D, Q, N and E.
Y is a moiety comprising a sequence having at least 80% identity with SEQ ID NO: 2, a total number of H, R, K, D, Q, N and E in X is less than 33.
characterized in that the mutant has at least 1, 2, 3, 4, 5, 6, 7, 8, or 9 D in X mutated to S, and/or at least 1, 2, 3, 4, 5, or 6 E in X mutated to T.

In some embodiments, at least 20% amino acids of X are selected from R, K, N, D, Q, E, and H, at least 30% amino acids of X are selected from R, K, N, D, Q, E, and H, at most 90% amino acids of X are selected from R, K, N, D, Q, E, and H, and/or at most 80% amino acids of X are selected from R, K, N, D, Q, E, and H. In some embodiments, at least 20% amino acids of X are selected from R, K, N, D, Q, E, and H. In some embodiments, at least 30% amino acids of X are selected from R, K, N, D, Q, E, and H. In some embodiments, at most 90% amino acids of X are selected from R, K, N, D, Q, E, and H. In some embodiments, at most 80% amino acids of X are selected from R, K, N, D, Q, E, and H. In some embodiments. X comprises a sequence having at least 70% identity with SEQ ID NO: 1. In some embodiments, the total number of H, R, K. D, Q, N and E in X is less than 30, the total number of H, R, K, D, Q, N and E in X is less than 25, or the total number of H, R, K, D, Q, N and E in X is less than 20. In some embodiments, the total number of H, R, K, D, Q, N and E in X is less than 30. In some embodiments, the total number of H, R, K, D, Q, N and E in X is less than 25. In some embodiments, the total number of H, R, K, D, Q, N and E in X is less than 20. In some embodiments, a total number of hydrophilic amino acids in X is more than 10, a total number of hydrophilic amino acids in X is more than 15, a total number of hydrophilic amino acids in X is more than 20, a total number of hydrophilic amino acids in X is more than 25, a total number of hydrophobic amino acids in X is no more than 15, and/or the total number of hydrophobic amino acids in X is no more than 10. In some embodiments, a total number of hydrophilic amino acids in X is more than 10. In some embodiments, a total number of hydrophilic amino acids in X is more than 15. In some embodiments, a total number of hydrophilic amino acids in X is more than 20. In some embodiments, a total number of hydrophilic amino acids in X is more than 25. In some embodiments, a total number of hydrophobic amino acids in X is no more than 15. In some embodiments, the total number of hydrophobic amino acids in X is no more than 10.

In some embodiments, Y comprises a sequence having at least 90% identity with SEQ ID NO: 2, or Y comprises a sequence of SEQ ID NO: 2. In some embodiments, Y comprises a sequence having at least 90% identity with SEQ ID NO: 2. In some embodiments, Y comprises a sequence of SEQ ID NO: 2. In some embodiments, at least 50% amino acids of Y are selected from I, V, L, F, C, M, and A.

In another aspect, provided is a mutant of artificial polypeptide of formula (IV),

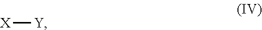

wherein X is a moiety comprising 40 to 65 amino acids, at least 50% of which are selected from H, R, K, D, Q, N and E.
Y is a moiety comprising a sequence having at least 80% identity with SEQ ID NO: 2,
X comprises a sequence having at most 90% identity with SEQ ID NO: 1, characterized in that the mutant has at least 1, 2, 3, 4, 5, 6, 7, 8, or 9 D in X mutated to S, and/or at least 1, 2, 3, 4, 5, or 6 E in X mutated to T.

In some embodiments, at least 20% amino acids of X are selected from R, K, N, D, Q, E, and H, at least 30% amino acids of X are selected from R, K, N, D, Q, E, and H, at most 90% amino acids of X are selected from R, K, N, D, Q, E, and H, and/or at most 80% amino acids of X are selected from R, K, N, D, Q, E, and H. In some embodiments, at least 20% amino acids of X are selected from R, K, N, D, Q, E, and H. In some embodiments, at least 30% amino acids of X are selected from R, K, N, D, Q, E, and H. In some embodiments, at most 90% amino acids of X are selected from R, K, N, D, Q, E, and H. In some embodiments, at most 80% amino acids of X are selected from R, K, N, D, Q, E, and H. In some embodiments, X comprises a sequence having at least 70% identity with SEQ ID NO: 1. In some embodiments, a total number of hydrophilic amino acids in X is more than 10, a total number of hydrophilic amino acids in X is more than 15, a total number of hydrophilic amino acids in X is more than 20, a total number of hydrophilic amino acids in X is more than 25, a total number of hydrophobic amino acids in X is no more than 15, and/or the total number of hydrophobic amino acids in X is no more than 10. In some embodiments, a total number of hydrophilic amino acids in X is more than 10. In some embodiments, a total number of hydrophilic amino acids in X is more than 15. In some embodiments, a total number of hydrophilic amino acids in X is more than 20. In some embodiments, a total number of hydrophilic amino acids in X is more than 25. In some embodiments, a total number of hydrophobic amino acids in X is no more than 15. In some embodiments, the total number of hydrophobic amino acids in X is no more than 10.

In some embodiments, Y comprises a sequence having at least 90% identity with SEQ ID NO: 2, or Y comprises a sequence of SEQ ID NO: 2. In some embodiments, Y comprises a sequence having at least 90% identity with SEQ ID NO: 2. In some embodiments, Y comprises a sequence of SEQ ID NO: 2. In some embodiments, at least 50% amino acids of Y are selected from I, V, L, F. C, M, and A.

In another aspect, provided is a mutant of artificial polypeptide of formula (V),

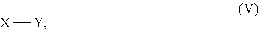

wherein X is a moiety comprising a sequence having at least 80% identity with SEQ ID NO: 1,
Y is a moiety comprising 10 to 30 amino acids, wherein Y comprises a sequence having at least 10 continuous AAs of SEQ ID NO: 2.
characterized in that the mutant has at least 1, 2, 3, 4, 5, 6, 7, 8, or 9 D in X mutated to S, and/or at least 1, 2, 3, 4, 5, or 6 E in X mutated to T.

In some embodiments, at least 20% amino acids of X are selected from R, K, N, D, Q, E, and H, at least 30% amino acids of X are selected from R, K, N, D, Q, E, and H, at most 90% amino acids of X are selected from R, K, N, D, Q, E, and H, and/or at most 80% amino acids of X are selected from R, K, N, D, Q, E, and H. In some embodiments, at least 20% amino acids of X are selected from R. K, N, D, Q, E, and H. In some embodiments, at least 30% amino acids of X are selected from R, K, N, D, Q, E, and H. In some embodiments, at most 90% amino acids of X are selected from R, K, N, D, Q, E, and H. In some embodiments, at most 80% amino acids of X are selected from R, K, N, D, Q, E, and H. In some embodiments, X comprises a sequence having at least 70% identity with SEQ ID NO: 1.

In some embodiments, Y comprises 10 to 25 amino acids, Y comprises 10 to 20 amino acids, or Y comprises 10 to 15 amino acids. In some embodiments, Y comprises 10 to 25 amino acids. In some embodiments, Y comprises 10 to 20 amino acids. In some embodiments, Y comprises 10 to 15 amino acids. In some embodiments, Y comprises a sequence having at most 80% identity with SEQ ID NO: 2, Y comprises a sequence having at most 70% identity with SEQ ID NO: 2, or Y comprises a sequence having at most 50% identity with SEQ ID NO: 2. In some embodiments, Y comprises a sequence having at most 80% identity with SEQ ID NO: 2. In some embodiments, Y comprises a sequence having at most 70% identity with SEQ ID NO: 2. In some embodiments, Y comprises a sequence having at most 50% identity with SEQ ID NO: 2.

In another aspect, provided is a formulation comprising an artificial polypeptide of formula (VI) or (VII) or a mutant of artificial polypeptide of (I), (II), (III), (IV) or (V) as described hereinbefore and a pharmaceutically acceptable excipient.

In another aspect, provided is a method for treating or preventing pain in a subject in need thereof, comprising administering to the subject an effective amount of an artificial polypeptide of formula (I):

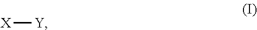

wherein X is a moiety comprising a sequence having at least 80% identity with SEQ ID NO: 1, and
Y is a moiety comprising 10 to 50 amino acids, at least 50% of which are selected from I, V, L, F. C, M, and A:
wherein Y comprises a total number of Cysteine (C) of less than 5.

In some embodiments. X comprises a sequence having at least 90% identity with SEQ ID NO: 1, X comprises a sequence having at least 95% identity with SEQ ID NO: 1, or X comprises a sequence of SEQ ID NO: 1. In some embodiments, X comprises a sequence having at least 90% identity with SEQ ID NO: 1. In some embodiments, X comprises a sequence having at least 95% identity with SEQ ID NO: 1. In some embodiments, X comprises a sequence of SEQ ID NO: 1. In some embodiments, at least 50% amino acids of X are selected from R, K, N, D, Q, E, and H. In some embodiments, X comprises a sequence having at least 90% identity with any one selected from SEQ ID NOs: 81-83.

In some embodiments, Y comprises a total number of Cysteine (C) of less than 4, Y comprises a total number of Cysteine (C) of less than 3, or Y comprises a total number of Cysteine (C) of less than 2. In some embodiments, Y comprises a total number of Cysteine (C) of less than 4. In some embodiments, Y comprises a total number of Cysteine (C) of less than 3. In some embodiments, Y comprises a total number of Cysteine (C) of less than 2. In some embodiments, a total number of hydrophobic amino acids in Y is more than 8, the total number of hydrophobic amino acids in Y is more than 12, the total number of hydrophobic amino acids in Y is more than 15, and/or a total number of hydrophilic amino acids in Y is no more than 5. In some embodiments, a total number of hydrophobic amino acids in Y is more than 8. In some embodiments, the total number of hydrophobic amino acids in Y is more than 12. In some embodiments, the total number of hydrophobic amino acids in Y is more than 15. In some embodiments, a total number of hydrophilic amino acids in Y is no more than 5. In some embodiments, Y comprises a sequence having at least 90% identity with any one selected from SEQ ID NOs: 3-15, 58-61 and 84-85, or Y comprises a sequence having at least 90% identity with any one selected from SEQ ID NOs: 3-18, 58-61 and 84-85. In some embodiments, Y comprises a sequence having at least 90% identity with any one selected from SEQ ID NOs: 3-15, 58-61 and 84-85. In some embodiments, Y comprises a sequence having at least 90% identity with any one selected from SEQ ID NOs: 3-18, 58-61 and 84-85.

In some embodiments, the polypeptide comprises a sequence having at least 90% identity with any one selected from SEQ ID NOs: 29-41, 54-57, 75 and 91-95.

In another aspect, provided is a method for treating or preventing pain in a subject in need thereof, comprising administering to the subject an effective amount of an artificial polypeptide of formula (II):

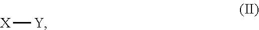

wherein X is a moiety comprising a sequence having at least 80% identity with SEQ ID NO: 1, and
Y is a moiety comprising 10 to 50 amino acids, at least 50% of which are selected from I, V, L, F, C. M, and A:
wherein Y comprises a sequence having at most 90% identity with SEQ ID NO: 2.

In some embodiments. X comprises a sequence having at least 90% identity with SEQ ID NO: 1. X comprises a sequence having at least 95% identity with SEQ ID NO: 1, or X comprises a sequence of SEQ ID NO: 1. In some embodiments. X comprises a sequence having at least 90% identity with SEQ ID NO: 1. In some embodiments. X comprises a sequence having at least 95% identity with SEQ ID NO: 1. In some embodiments. X comprises a sequence of SEQ ID NO: 1. In some embodiments, at least 50% amino acids of X are selected from R, K, N, D, Q, E, and H.

In some embodiments. Y comprises a sequence having at most 80% identity with SEQ ID NO: 2. Y comprises a sequence having at most 70% identity with SEQ ID NO: 2, or Y comprises a sequence having at most 50% identity with SEQ ID NO: 2. In some embodiments. Y comprises a sequence having at most 80% identity with SEQ ID NO: 2. In some embodiments. Y comprises a sequence having at most 70% identity with SEQ ID NO: 2. In some embodiments. Y comprises a sequence having at most 50% identity with SEQ ID NO: 2. In some embodiments, a total number of hydrophobic amino acids in Y is more than 8, the total number of hydrophobic amino acids in Y is more than 12, the total number of hydrophobic amino acids in Y is more than 15, and/or a total number of hydrophilic amino acids in Y is no more than 5. In some embodiments, a total number of hydrophobic amino acids in Y is more than 8. In some embodiments, the total number of hydrophobic amino acids in Y is more than 12. In some embodiments, the total number of hydrophobic amino acids in Y is more than 15. In some embodiments, a total number of hydrophilic amino acids in Y is no more than 5. In some embodiments. Y comprises a sequence having at least 90% identity with any one selected from SEQ ID NOs: 16-18, or Y comprises a sequence having at least 95% identity with any one selected from SEQ ID NOs: 16-18. In some embodiments. Y comprises a sequence having at least 90% identity with any one selected from SEQ ID NOs: 16-18. In some embodiments. Y comprises a sequence having at least 95% identity with any one selected from SEQ ID NOs: 16-18.

In some embodiments, the polypeptide comprises a sequence having at least 90% identity with any one selected from SEQ ID NOs: 42-44.

In another aspect, provided is a method for treating or preventing pain in a subject in need thereof, comprising administering to the subject an effective amount of an artificial polypeptide of formula (III):

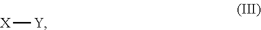

wherein X is a moiety comprising 40 to 65 amino acids, at least 50% of which are selected from H, R, K, D,

### Q, N and E; and

Y is a moiety comprising a sequence having at least 80% identity with SEQ ID NO: 2,
wherein a total number of H, R, K, D, Q, N and E in X is less than 33.

In some embodiments. Y comprises a sequence having at least 90% identity with SEQ ID NO: 2. Y comprises a sequence having at least 95% identity with SEQ ID NO: 2, or Y comprises a sequence of SEQ ID NO: 2. In some embodiments. Y comprises a sequence having at least 90% identity with SEQ ID NO: 2. In some embodiments. Y comprises a sequence having at least 95% identity with SEQ ID NO: 2. In some embodiments. Y comprises a sequence of SEQ ID NO: 2. In some embodiments, at least 50% amino acids of Y are selected from I, V, L, F, C, M, and A.

In some embodiments, wherein the total number of H, R, K, D, Q, N and E in X is less than 30, the total number of H, R, K, D, Q, N and E in X is less than 25, or the total number of H, R, K, D, Q, N and E in X is less than 20. In some embodiments, the total number of H, R, K, D, Q, N and E in X is less than 30. In some embodiments, the total number of H, R, K, D, Q, N and E in X is less than 25. In some embodiments, the total number of H, R, K, D, Q, N and E in X is less than 20. In some embodiments, a total number of hydrophilic amino acids in X is more than 10, a total number of hydrophilic amino acids in X is more than 15, a total number of hydrophilic amino acids in X is more than 20, a total number of hydrophilic amino acids in X is more than 25, a total number of hydrophobic amino acids in X is no more than 15, and/or the total number of hydrophobic amino acids in X is no more than 10. In some embodiments, a total number of hydrophilic amino acids in X is more than 10. In some embodiments, a total number of hydrophilic amino acids in X is more than 15. In some embodiments, a total number of hydrophilic amino acids in X is more than 20. In some embodiments, a total number of hydrophilic amino acids in X is more than 25. In some embodiments, a total number of hydrophobic amino acids in X is no more than 15. In some embodiments, the total number of hydrophobic amino acids in X is no more than 10. In some embodiments. X comprises a sequence having at least 90% identity with any one selected from SEQ ID NOs: 23-27, or X comprises a sequence having at least 95% identity with any one selected from SEQ ID NOs: 23-27. In some embodiments. X comprises a sequence having at least 90% identity with any one selected from SEQ ID NOs: 23-27. In some embodiments. X comprises a sequence having at least 95% identity with any one selected from SEQ ID NOs: 23-27.

In some embodiments, the polypeptide comprises a sequence having at least 90% identity with any one selected from SEQ ID NOs: 49-53.

In another aspect, provided is a method for treating or preventing pain in a subject in need thereof, comprising administering to the subject an effective amount of an artificial polypeptide of formula (IV):

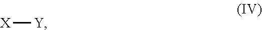

wherein X is a moiety comprising 40 to 65 amino acids, at least 50% of which are selected from H, R, K, D,

### Q, N and E; and

Y is a moiety comprising a sequence having at least 80% identity with SEQ ID NO: 2, wherein X comprises a sequence having at most 90% identity with SEQ ID NO: 1.

In some embodiments. X comprises a sequence having at most 80% identity with SEQ ID NO: 1. X comprises a sequence having at most 70% identity with SEQ ID NO: 1, or X comprises a sequence having at most 50% identity with SEQ ID NO: 1. In some embodiments. X comprises a sequence having at most 80% identity with SEQ ID NO: 1. In some embodiments. X comprises a sequence having at most 70% identity with SEQ ID NO: 1. In some embodiments. X comprises a sequence having at most 50% identity with SEQ ID NO: 1. In some embodiments, a total number of hydrophilic amino acids in X is more than 10, a total number of hydrophilic amino acids in X is more than 15, a total number of hydrophilic amino acids in X is more than 20, a total number of hydrophilic amino acids in X is more than 25, a total number of hydrophobic amino acids in X is no more than 15, and/or the total number of hydrophobic amino acids in X is no more than 10. In some embodiments, a total number of hydrophilic amino acids in X is more than 10. In some embodiments, a total number of hydrophilic amino acids in X is more than 15. In some embodiments, a total number of hydrophilic amino acids in X is more than 20. In some embodiments, a total number of hydrophilic amino acids in X is more than 25. In some embodiments, a total number of hydrophobic amino acids in X is no more than 15. In some embodiments, the total number of hydrophobic amino acids in X is no more than 10. In some embodiments. X comprises a sequence having at least 90% identity with any one selected from SEQ ID NOs: 19-22 and 76-79, or X comprises a sequence having at least 95% identity with any one selected from SEQ ID NOs: 19-22 and 76-79. In some embodiments. X comprises a sequence having at least 90% identity with any one selected from SEQ ID NOs: 19-22 and 76-79. In some embodiments. X comprises a sequence having at least 95% identity with any one selected from SEQ ID NOs: 19-22 and 76-79.

In some embodiments. Y comprises a sequence having at least 90% identity with SEQ ID NO: 2. Y comprises a sequence having at least 95% identity with SEQ ID NO: 2, or Y comprises a sequence of SEQ ID NO: 2. In some embodiments. Y comprises a sequence having at least 90% identity with SEQ ID NO: 2. In some embodiments. Y comprises a sequence having at least 95% identity with SEQ ID NO: 2. In some embodiments. Y comprises a sequence of SEQ ID NO: 2. In some embodiments, at least 50% amino acids of Y are selected from I, V, L, F, C, M, and A.

In some embodiments, the polypeptide comprises a sequence having at least 90% identity with any one selected from SEQ ID NOs: 45-48 and 86-89.

In another aspect, provided is a method for treating or preventing pain in a subject in need thereof, comprising administering to the subject an effective amount of an artificial polypeptide of formula (V):

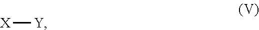

wherein X is a moiety comprising a sequence having at least 80% identity with SEQ ID NO: 1, and Y is a moiety comprising 10 to 30 amino acids, wherein Y comprises a sequence having at least 10 continuous AAs of SEQ ID NO: 2.

In some embodiments. X comprises a sequence having at least 90% identity with SEQ ID NO: 1. X comprises a sequence having at least 95% identity with SEQ ID NO: 1, or X comprises a sequence of SEQ ID NO: 1. In some embodiments. X comprises a sequence having at least 90% identity with SEQ ID NO: 1. In some embodiments. X comprises a sequence having at least 95% identity with SEQ ID NO: 1. In some embodiments. X comprises a sequence of SEQ ID NO: 1. In some embodiments, at least 50% amino acids of X are selected from R, K, N, D, Q, E, and H. In some embodiments. X comprises a sequence having at least 80% identity with SEQ ID NO: 96. X comprises a sequence having at least 90% identity with SEQ ID NO: 96. X comprises a sequence having at least 95% identity with SEQ ID NO: 96, or X comprises a sequence of SEQ ID NO: 96. In some embodiments. X comprises a sequence having at least 80% identity with SEQ ID NO: 96. In some embodiments. X comprises a sequence having at least 90% identity with SEQ ID NO: 96. In some embodiments. X comprises a sequence having at least 95% identity with SEQ ID NO: 96. In some embodiments. X comprises a sequence of SEQ ID NO: 96.

In some embodiments. Y comprises 10 to 25 amino acids. Y comprises 10 to 20 amino acids, or Y comprises 10 to 15 amino acids. In some embodiments. Y comprises 10 to 25 amino acids. In some embodiments. Y comprises 10 to 20 amino acids. In some embodiments. Y comprises 10 to 15 amino acids.

In some embodiments. Y comprises a sequence having at most 80% identity with SEQ ID NO: 2. Y comprises a sequence having at most 70% identity with SEQ ID NO: 2, or Y comprises a sequence having at most 50% identity with SEQ ID NO: 2. In some embodiments. Y comprises a sequence having at most 80% identity with SEQ ID NO: 2. In some embodiments. Y comprises a sequence having at most 70% identity with SEQ ID NO: 2. In some embodiments. Y comprises a sequence having at most 50% identity with SEQ ID NO: 2. In some embodiments. Y comprises a sequence having at least 80% identity with any one selected from SEQ ID NOs: 97-103. Y comprises a sequence having at least 90% identity with any one selected from SEQ ID NOs: 97-103. Y comprises a sequence having at least 95% identity with any one selected from SEQ ID NOs: 97-103, or Y comprises a sequence that is any one selected from SEQ ID NOs: 97-103. In some embodiments. Y comprises a sequence having at least 80% identity with any one selected from SEQ ID NOs: 97-103. In some embodiments. Y comprises a sequence having at least 90% identity with any one selected from SEQ ID NOs: 97-103. In some embodiments. Y comprises a sequence having at least 95% identity with any one selected from SEQ ID NOs: 97-103. In some embodiments. Y comprises a sequence that is any one selected from SEQ ID NOs: 97-103.

In some embodiments, the polypeptide comprises a sequence having at least 90% identity with any one selected from SEQ ID NOs: 53-56 and 104-110.

In another aspect, provided is a method for treating or preventing pain in a subject in need thereof, comprising administering to the subject an effective amount of an artificial polypeptide of formula (VI) or (VII) or a mutant of artificial polypeptide of (I), (II). (III), (IV) or (V) as described hereinbefore.

In some embodiments, the pain is at least one selected from the group consisting of acute pain, chronic pain and transitional pain.

In some embodiments, the acute pain is at least one selected from the group consisting of pain associated with acute injuries, acute inflammatory pain and headache.

In some embodiments, the pain associated with acute injuries is acute postoperative pain.

In some embodiments, the chronic pain is at least one selected from the group consisting of nociceptive pain, neuropathic pain and mixed pain.

In some embodiments, the nociceptive pain is at least one selected from the group consisting of abdominal pain, anal fissure pain, bladder pain, complex regional pain syndrome, mastalgia, inhntenterospasm. bladder pain syndrome, arthralgia, musculoskeletal pain, muscle pain, myofascial pain syndrome, nociceptive bone pain, pain associated with pancreatitis, polymyalgia rheumatica, chronic postoperative pain, renal pain, somatic pain, teinodynia, desmalgia, chronic traumatic pain, pain associated with fracture and visceral pain.

In some embodiments, the mastalgia is cyclic mastalgia.

In some embodiments, the renal pain is renal colic.

In some embodiments, the pain associated with fracture is pain associated with spine fracture.

In some embodiments, the mixed pain is at least one selected from the group consisting of arthritis pain, back pain, cancer pain, toothache, fibromyalgia, chronic inflammatory pain, lumbago, cervicodynia and eye pain.

In some embodiments, the arthritis pain is at least one selected from the group consisting of osteoarthritis pain, rheumatoid arthritis pain and gout pain.

In some embodiments, the cancer pain is cancer pain associated with tumor.

In some embodiments, the cancer pain associated with tumor is at least one selected from the group consisting of nerve pain associated with cancer, bone pain associated with cancer, soft tissue pain associated with cancer and referred pain associated with cancer.

In some embodiments, the bone pain associated with cancer is at least one selected from the group consisting of bone pain associated with bone cancers and bone pain associated with bone metastatic tumors.

In some embodiments, the bone pain associated with bone cancers is at least one selected from the group consisting of bone pain associated with chondrosarcoma, bone pain associated with Ewing's sarcoma, bone pain associated with malignant fibrous histiocytoma of bone, bone pain associated with osteosarcoma and bone pain associated with fibrocartilaginous mesenchymoma of bone.

In some embodiments, the cancer pain is cancer pain associated with diagnostic and/or therapeutic procedures.

In some embodiments, the cancer pain associated with diagnostic and/or therapeutic procedures is phantom pain associated with cancer. In some embodiments, the cancer pain associated with diagnostic and/or therapeutic procedures is cancer pain associated with chemotherapy. In some embodiments, the cancer pain associated with chemotherapy is cancer pain associated with chemotherapy-induced peripheral neuropathy (CIPN). In some embodiments, the chemotherapy is performed by administration of a cytotoxic agent to a subject in need thereof. In some embodiments, the cytotoxic agent is at least one selected from the group consisting of platinum anticancer agents (e.g., cisplatin, carboplatin, nedaplatin and oxaliplatin), vinca alkaloids (e.g., vinblastine, vinorelbine, vincristine and vindesine), taxanes (e.g., paclitaxel and docetaxel) and proteasome or angiogenesis inhibitors (e.g., bortezomib, carfilzomib and thalidomide).

In some embodiments, the eye pain is at least one selected from the group consisting of ocular nociceptive pain and ocular neuropathic pain.

In some embodiments, the ocular nociceptive pain is at least one selected from the group consisting of eye pain associated with injury, eye pain associated with surgery, eye pain associated with contact lenses wearing and eye pain associated with irritation of foreign bodies.

In some embodiments, the ocular neuropathic pain is at least one selected from the group consisting of eye pain associated with allergies, eye pain associated with infection, eye pain associated with inflammation, eye pain associated with chronic ocular surface disease, post-surgical ocular neuropathic pain, eye pain associated with toxic keratopathy, eye pain associated with radiation, eye pain associated with ultraviolet light exposure, eye pain associated with a systemic neuropathy, traumatic ocular neuropathic pain, eye pain with trigeminal neuralgia and eye pain associated with fibromyalgia.

In some embodiments, the ocular neuropathic pain is at least one selected from the group consisting of corneal neuralgia, conjunctival neuralgia, optic nerve neuralgia, extraocular muscle neuralgia, orbital neuralgia and eyelid neuralgia.

In some embodiments, the neuropathic pain is at least one selected from the group consisting of central neuropathic pain and peripheral neuropathic pain.

In some embodiments, the central neuropathic pain is at least one selected from the group consisting of post-stroke neuropathic pain, syringomyelia pain, neuropathic pain associated with ischemic myelopathy, neuropathic pain associated with oppressed myelopathy, neuropathic pain associated with radiation myelopathy, neuropathic pain associated with spinal cord injury, neuropathic pain associated with multiple sclerosis, neuropathic pain associated with Parkinson's disease, phantom limb pain and neuropathic pain associated with myelitis.

In some embodiments, the peripheral neuropathic pain is at least one selected from the group consisting of peripheral neuropathic pain associated with metabolic disorder or ischemia, peripheral neuropathic pain associated with infection, peripheral neuropathic pain associated with nerve or nerve root compression, peripheral neuropathic pain associated with chemotherapy, peripheral neuropathic pain associated with radiotherapy, stump pain, peripheral neuropathic pain associated with neuropathy due to tumor compression or infiltration, peripheral neuropathic pain associated with alcoholic polyneuropathy, peripheral neuropathic pain associated with dystrophic neuropathy, peripheral neuropathic pain associated with toxic peripheral neuropathy and peripheral neuropathic pain associated with immune neuropathy.

In some embodiments, the peripheral neuropathic pain associated with metabolic disorder or ischemia is at least one selected from the group consisting of diabetic peripheral neuropathic pain and ischemic peripheral neuropathic pain.

In some embodiments, the peripheral neuropathic pain associated with infection is at least one selected from the group consisting of peripheral neuropathic pain associated with viral infection and peripheral neuropathic pain associated with spirochetal infection.

In some embodiments, the peripheral neuropathic pain associated with viral infection is at least one selected from the group consisting of post-herpetic neuralgia and peripheral neuropathic pain associated with HIV infection.

In some embodiments, the peripheral neuropathic pain associated with spirochetal infection is peripheral neuropathic pain associated with syphilis infection.

In some embodiments, the peripheral neuropathic pain associated with nerve or nerve root compression is at least one selected from the group consisting of sciatica, trigeminal neuralgia, glossopharyngeal neuralgia, neuropathic pain associated with radiculoneuropathy and neuropathic pain associated with entrapment neuropathy.

In another aspect, provided is a method for treating or preventing peripheral neuropathic pain in a subject in need thereof, comprising administering to the subject an effective amount of a polypeptide having at least 70% identity with any one selected from SEQ ID NO: 28. SEQ ID NOs: 57 and 62-74 or a fragment or variant thereof.

In some embodiments, the polypeptide is a polypeptide of any one of SEQ ID NO: 28. SEQ ID NOs: 57 and 62-74 or a fragment or variant thereof, the polypeptide is a polypeptide of any one of SEQ ID NO: 28. SEQ ID NOs: 57 and 62-74, the polypeptide is a polypeptide of any one of SEQ ID NOs: 28 and 62-74, or the polypeptide is a polypeptide of SEQ ID NO: 28. In some embodiments, the polypeptide is a polypeptide of any one of SEQ ID NO: 28. SEQ ID NOs: 57 and 62-74 or a fragment or variant thereof. In some embodiments, the polypeptide is a polypeptide of any one of SEQ ID NO: 28. SEQ ID NOs: 57 and 62-74. In some embodiments, the polypeptide is a polypeptide of any one of SEQ ID NOs: 28 and 62-74. In some embodiments, the polypeptide is a polypeptide of SEQ ID NO: 28.

In some embodiments, the peripheral neuropathic pain is at least one selected from the group consisting of peripheral neuropathic pain associated with metabolic disorder or ischemia, peripheral neuropathic pain associated with infection, peripheral neuropathic pain associated with nerve or nerve root compression, peripheral neuropathic pain associated with chemotherapy, peripheral neuropathic pain associated with radiotherapy, stump pain, peripheral neuropathic pain associated with neuropathy due to tumor compression or infiltration, peripheral neuropathic pain associated with alcoholic polyneuropathy, peripheral neuropathic pain associated with dystrophic neuropathy, peripheral neuropathic pain associated with toxic peripheral neuropathy and peripheral neuropathic pain associated with immune neuropathy.

In some embodiments, the peripheral neuropathic pain associated with metabolic disorder or ischemia is at least one selected from the group consisting of diabetic peripheral neuropathic pain and ischemic peripheral neuropathic pain.

In some embodiments, the peripheral neuropathic pain associated with infection is at least one selected from the group consisting of peripheral neuropathic pain associated with viral infection and peripheral neuropathic pain associated with spirochetal infection.

In some embodiments, the peripheral neuropathic pain associated with viral infection is at least one selected from the group consisting of post-herpetic neuralgia and peripheral neuropathic pain associated with HIV infection.

In some embodiments, the peripheral neuropathic pain associated with spirochetal infection is peripheral neuropathic pain associated with syphilis infection.

In some embodiments, the peripheral neuropathic pain associated with nerve or nerve root compression is at least one selected from the group consisting of sciatica, trigeminal neuralgia, glossopharyngeal neuralgia, neuropathic pain associated with radiculoneuropathy and neuropathic pain associated with entrapment neuropathy.

In some embodiments, the subject is a mammal. In some embodiments, the subject is any one selected from the group consisting of a human, primate, rodent, canine, feline, equine, ovine and porcine. In some embodiments, the subject is a pet. In some embodiments, the subject is any one selected from the group consisting of fish, frog, salamander, reptile (e.g., turtle, lizard, snake and iguana), bird (e.g., a parrot), mice, rat, guinea pig, gerbil, hamster, chinchilla, rabbit, ferret, cat, dog and pig.

In some embodiments, the artificial polypeptide of formula (I), (II), (III), (IV), (V), (VI), or (VII), or the mutant of artificial polypeptide of (I), (II), (III), (IV) or (V), or the polypeptide having at least 70% identity with any one selected from SEQ ID NO: 28, SEQ ID NOs: 57 and 62-74 or a fragment or variant thereof is administered in the form of a formulation comprising the same and a pharmaceutically acceptable excipient.

In some embodiments, the formulation is in a form suitable for oral administration. In some embodiments, the formulation is in a form of a tablet, capsule, pill, powder, granule, solution, suspension or emulsion.

In some embodiments, the formulation is in a form suitable for parenteral administration. In some embodiments, formulation is in a form of a sterile solution, suspension, emulsion, gel (e.g., injectable hydrogel) or a sterile powder for injection.

In some embodiments, the formulation is in a form suitable for topical administration. In some embodiments, the formulation is in a form of a spray, aerosol, powder for inhalation, ointment, cream, patch, suppository, paste, pellicle or gel.

In some embodiments, the formulation is in a form of sustained, controlled or delayed release formulation. In some embodiments, the formulation is in a form of a matrix tablet, compressed tablet with mixed particles having various rate of release, osmotic pump tablet, sustained or controlled release capsule or microcapsule, sustained or controlled release implant, sustained or controlled release particle, microparticle or microsphere, enteric coated capsule or tablet, colon-located formulation, gel (e.g., hydrogel), liposome, ion-exchange resin, floating formulation, bio-adhesive formulation, stimuli inducing release formulation and pulsatile drug delivery system.

In some embodiments, the formulation is a formulation for use in human.

In some embodiments, the formulation is a veterinary formulation.

In another aspect, provided is an artificial polypeptide of formula (VI) or (VII), or a mutant of artificial polypeptide of (I), (II), (III), (IV) or (V), for use in therapy.

In another aspect, provided is an artificial polypeptide of formula (I), (II), (III), (IV), (V), (VI), or (VII), or a mutant of artificial polypeptide of (I), (II), (III), (IV) or (V), for use in treating or preventing pain in a subject in need thereof.

In another aspect, provided is a polypeptide having at least 70% identity with any one selected from SEQ ID NO: 28, SEQ ID NOs: 57 and 62-74 or a fragment or variant thereof, for use in treating or preventing peripheral neuropathic pain in a subject in need thereof.

In another aspect, provided is use of an artificial polypeptide of formula (I), (II), (III), (IV), (V), (VI), or (VII), or a mutant of artificial polypeptide of (I), (II), (III), (IV) or (V) for the preparation of a medicament for treating or preventing pain in a subject in need thereof.

In another aspect, provided is use of a polypeptide having at least 70% identity with any one selected from SEQ ID NO: 28, SEQ ID NOs: 57 and 62-74 or a fragment or variant thereof for the preparation of a medicament for treating or preventing peripheral neuropathic pain in a subject in need thereof.

In some embodiments, the medicament is a medicament for use in human.

In some embodiments, the medicament is a veterinary medicament.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

Various features of this disclosure are set forth with particularity in the appended claims. A better understanding of the features and advantages of the present disclosure will be obtained by reference to the following detailed description that sets forth illustrative embodiments, in which the principles of the invention are utilized, and the accompanying drawings of which:

FIG. 1 shows the 50% PWT values of the rats from both placebo group and treatment groups (SEQ ID NOs: 29, 57, 75, 49 and 28, s.c, or i.v., single administration) obtained from the mechanical allodynia tests of Examples 1-3.

FIG. 2 shows the 50% PWT values of the mice from various treatment groups (SEQ ID NOs: 28, 29, 57, 86, 89, 90, 91, 93, 94, 95, 104, 106, 107, 108, 109 and 110, i.v., single administration) obtained from the mechanical allodynia tests of Example 4.

FIG. 3 shows the 50% PWT values of the mice from the Sham group, the vehicle group and various treatment groups (SEQ ID NOs: 29, 90 and 93, i.v., single administration) obtained from the mechanical allodynia tests of Example 5.

FIG. 4 shows the 50% PWT values of the rats from the Sham group, the model group and the treatment group (SEQ ID NO: 29, i.v., continuous administration) obtained from the mechanical allodynia tests of Example 6.

FIG. 5 shows the 50% PWT values of the mice from the control group, the model group, the treatment group (qd) (SEQ ID NO: 29, i.p., qd) and the treatment group (bid) (SEQ ID NO: 29, i.p., bid) obtained from the mechanical allodynia tests of Example 7.

FIG. 6 shows the numbers of wipes over the eyes of the mice from the control group, the model group and the test substance group (SEQ ID NO: 29, via eye drops, tid) obtained from the scratching behavior test of Example 8.

The patent or application file contains at least one drawing executed in color. Copies of this patent or patent application publication with color drawings will be provided by the Office upon request and payment of the necessary fec.

### DETAILED DESCRIPTION

### Definition

As used herein, the following terms have the meanings ascribed to them unless specified otherwise.

As used in the specification and claims, the singular forms “a,” “an,” and “the” include plural references unless the context clearly dictates otherwise. For example, the term “a cell” includes a plurality of cells, including mixtures thereof.

As used herein, the terms “comprise”, “include”, “contain” and variations thereof are intended to mean open-ended transitional phrases that do not exclude the possibilities of additional substances or methods. When such terms are used to describe a certain pharmaceutical composition, formulation, kit, use or method of the present disclosure, it also encompasses the situation that the pharmaceutical composition, formulation, kit, use or method consists of the recited substances or methods. For instance, the expression “the solvents comprise water” also includes the situation wherein the solvents are consisting of water, i.e., the solvents contain water exclusively. In the context of this disclosure, the term “consisting of′ is intended to mean a close-ended transitional phrase, which excludes the possibilities of additional substances or methods.

As used herein, ranges as recited in this disclosure are intended to explicitly disclose each of the endpoints of the range and each integer included in the range, unless otherwise indicated. For example, “X is a moiety comprising 40 to 65 amino acids” means that the number of amino acids in the moiety of X can be 40, 41, 42, 43, 44, 45, 46, 47, 48, 49, 50, 51, 52, 53, 54, 55, 56, 57, 58, 59, 60, 61, 62, 63, 64 or 65. For another example, “Y is a moiety comprising 10 to 50 amino acids” means that that the number of amino acids in moiety of Y can be 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50. Additionally, any sub-ranges consisting of these integers are intended to be included within the scope of this disclosure. Accordingly, “X is a moiety comprising 40 to 65 amino acids” is regarded as explicitly disclosing the sub-ranges such as “X is a moiety comprising 41 to 64 amino acids”, “X is a moiety comprising 42 to 63 amino acids”, “X is a moiety comprising 43 to 62 amino acids” . . . , etc.

The term “about” or “approximately” herein means within an acceptable error range of the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, up to 10%, up to 5%, or up to 1% of a given value. Where particular values are described in the application and claims, unless otherwise stated, the term “about” or “approximately” meaning within an acceptable error range for the particular value should be assumed.

The term “optional (ly)” means that the subsequently described event of circumstances may or may not occur, and that the description includes instances where the event or circumstance occurs and instances in which it does not.

The terms “polypeptide,” “peptide,” and “protein” are used interchangeably herein to refer to polymers of amino acids of any length. The polymer may be linear or branched, it may comprise modified amino acids, and it may be interrupted by non-amino acids. The terms also encompass an amino acid polymer that has been modified, for example, by disulfide bond formation, glycosylation, lipidation, acetylation, phosphorylation, or any other manipulation, such as conjugation with a labeling component.

The term “fragment” as used herein, when applied to a protein, refers to a truncated form of a native biologically active protein that may or may not retain at least a portion of the therapeutic and/or biological activity.

The term “variant” as used herein, when applied to a protein, refers to a protein with sequence homology to the native biologically active protein that retains at least a portion of the therapeutic and/or biological activity of the biologically active protein. For example, a variant protein may share at least 70%, 75%, 80%, 85%, 90%, 95%, 96%, 97%, 98% or 99% amino acid sequence identity as compared to the reference biologically active protein.

As used herein the term “amino acid” refers to either natural and/or unnatural or synthetic amino acids, including but not limited to glycine and both the D or L optical isomers, and amino acid analogs and peptidomimetics. Standard single or three letter codes are used to designate amino acids.

The term “natural L-amino acid” means the L optical isomer forms of glycine (G), proline (P), alanine (A), valine (V), leucine (L), isoleucine (I), methionine (M), cysteine (C), phenylalanine (F), tyrosine (Y), tryptophan (W), histidine (H), lysine (K), arginine (R), glutamine (Q), asparagine (N), glutamic acid (E), aspartic acid (D), serine(S), and threonine (T).

The terms “hydrophilic” and “hydrophobic” refer to the degree of affinity that a substance has with water. A hydrophilic substance has a strong affinity for water, tending to dissolve in, mix with, or be wetted by water, while a hydrophobic substance substantially lacks affinity for water, tending to repel and not absorb water and tending not to dissolve in or mix with or be wetted by water. Amino acids can be characterized based on their hydrophobicity. Examples of “hydrophilic amino acids” are arginine, lysine, threonine, alanine, asparagine, and glutamine. Of particular interest are the hydrophilic amino acids aspartate, glutamate, serine, and glycine. In some embodiments. “hydrophilic amino acids” refers to arginine, lysine, threonine, alanine, asparagine, glutamine, aspartate, glutamate, serine, and glycine. Examples of “hydrophobic amino acids” are tryptophan, tyrosine, phenylalanine, methionine, leucine, isoleucine, and valine. In some embodiments. “hydrophobic amino acids” refers to tryptophan, tyrosine, phenylalanine, methionine, leucine, isoleucine, and valine.

A “host cell” includes an individual cell or cell culture which can be or has been a recipient for the subject vectors. Host cells include progeny of a single host cell. The progeny may not necessarily be completely identical (in morphology or in genomic of total DNA complement) to the original parent cell.

A “chimeric” protein contains at least one fusion polypeptide comprising regions in a different position in the sequence than that which occurs in nature. The regions may normally exist in separate proteins and are brought together in the fusion polypeptide: or they may normally exist in the same protein but are placed in a new arrangement in the fusion polypeptide. A chimeric protein may be created, for example, by chemical synthesis, or by creating and translating a polynucleotide in which the peptide regions are encoded in the desired relationship.

“Conjugated”. “linked”. “fused” and “fusion” are used interchangeably herein. These terms refer to the joining together of two more chemical elements or components, by whatever means including chemical conjugation or recombinant means.

The terms “polynucleotides”. “nucleic acids”. “nucleotides” and “oligonucleotides” are used interchangeably. They refer to a polymeric form of nucleotides of any length, either deoxyribonucleotides or ribonucleotides, or analogs thereof. Polynucleotides may have any three-dimensional structure, and may perform any function, known or unknown. The following are non-limiting examples of polynucleotides: coding or non-coding regions of a gene or gene fragment, loci (locus) defined from linkage analysis, exons, introns. messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component.

The term “complement of a polynucleotide” denotes a polynucleotide molecule having a complementary base sequence and reverse orientation as compared to a reference sequence, such that it could hybridize with a reference sequence with complete fidelity.

The term “recombinant” as applied to a polynucleotide means that the polynucleotide is the product of various combinations of in vitro cloning, restriction and/or ligation steps, and other procedures that result in a construct that can potentially be expressed in a host cell.

The term “homology” or “homologous” refers to sequence similarity or interchangeability between two or more polynucleotide sequences or two or more polypeptide sequences. When using a program such as BestFit to determine sequence identity, similarity or homology between two different amino acid sequences, the default settings may be used, or an appropriate scoring matrix, such as blosum45 or blosum80, may be selected to optimize identity, similarity or homology scores. Preferably, polynucleotides that are homologous are those which hybridize under stringent conditions as defined herein and have at least 70%, preferably at least 80%, more preferably at least 90%, more preferably 95%, more preferably 97%, more preferably 98%, and even more preferably 99% sequence identity to those sequences.

The terms “percent identity” and “% identity.” as applied to polynucleotide sequences, refer to the percentage of residue matches between at least two polynucleotide sequences aligned using a standardized algorithm. Such an algorithm may insert, in a standardized and reproducible way, gaps in the sequences being compared in order to optimize alignment between two sequences, and therefore achieve a more meaningful comparison of the two sequences. Percent identity may be measured over the length of an entire defined polynucleotide sequence, for example, as defined by a particular SEQ ID number, or may be measured over a shorter length, for example, over the length of a fragment taken from a larger, defined polynucleotide sequence, for instance, a fragment of at least 45, at least 60, at least 90, at least 120, at least 150, at least 210 or at least 450 contiguous residues. Such lengths are exemplary only, and it is understood that any fragment length supported by the sequences shown herein, in the tables, figures or Sequence Listing, may be used to describe a length over which percentage identity may be measured.

“Percent (%) amino acid sequence identity.” with respect to the polypeptide sequences identified herein, is defined as the percentage of amino acid residues in a query sequence that are identical with the amino acid residues of a second, reference polypeptide sequence or a portion thereof, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST. BLAST-2. ALIGN or Megalign (DNASTAR) software. Those skilled in the art can determine appropriate parameters for measuring alignment, including any algorithms needed to achieve maximal alignment over the full length of the sequences being compared. Percent identity may be measured over the length of an entire defined polypeptide sequence, for example, as defined by a particular SEQ ID number, or may be measured over a shorter length, for example, over the length of a fragment taken from a larger, defined polypeptide sequence, for instance, a fragment of at least 15, at least 20, at least 30, at least 40, at least 50, at least 70 or at least 150 contiguous residues. Such lengths are exemplary only, and it is understood that any fragment length supported by the sequences shown herein, in the tables, figures or Sequence Listing, may be used to describe a length over which percentage identity may be measured.

A “vector” is a nucleic acid molecule, preferably self-replicating in an appropriate host, which transfers an inserted nucleic acid molecule into and/or between host cells. The term includes vectors that function primarily for insertion of DNA or RNA into a cell, replication of vectors that function primarily for the replication of DNA or RNA, and expression vectors that function for transcription and/or translation of the DNA or RNA. Also included are vectors that provide more than one of the above functions. An “expression vector” is a polynucleotide which, when introduced into an appropriate host cell, can be transcribed and translated into a polypeptide(s). An “expression system” usually connotes a suitable host cell comprised of an expression vector that can function to yield a desired expression product.

The term “t1:” as used herein means the terminal half-life calculated as In (2)/Ke1. Kel is the terminal elimination rate constant calculated by linear regression of the terminal linear portion of the log concentration vs, time curve. Half-life typically refers to the time required for half the quantity of an administered substance deposited in a living organism to be metabolized or eliminated by normal biological processes. The terms “t1?”. “terminal half-life”. “elimination half-life” and “circulating half-life” are used interchangeably herein.

The term “physiological conditions” refers to a set of conditions in a living host as well as in vitro conditions, including temperature, salt concentration, pH, that mimic those conditions of a living subject. A host of physiologically relevant conditions for use in in vitro assays have been established. Generally, a physiological buffer contains a physiological concentration of salt and is adjusted to a neutral pH ranging from about 6.5 to about 7.8, and preferably from about 7.0 to about 7.5. A variety of physiological buffers is listed in Sambrook et al. (1989). Physiologically relevant temperature ranges from about 25° C., to about 38° C., and preferably from about 35° C., to about 37° C.

The term “antagonist” as used herein, includes any molecule that partially or fully blocks, inhibits, or neutralizes a biological activity of a native polypeptide disclosed herein. Methods for identifying antagonists of a polypeptide may comprise contacting a native polypeptide with a candidate antagonist molecule and measuring a detectable change in one or more biological activities normally associated with the native polypeptide. In the context of the present disclosure, antagonists may include proteins, nucleic acids, carbohydrates, antibodies or any other molecules that decrease the effect of a biologically active protein.

The term “agonist” is used in the broadest sense and includes any molecule that mimics a biological activity of a native polypeptide disclosed herein. Suitable agonist molecules specifically include agonist antibodies or antibody fragments, fragments or amino acid sequence variants of native polypeptides, peptides, small organic molecules, etc. Methods for identifying agonists of a native polypeptide may comprise contacting a native polypeptide with a candidate agonist molecule and measuring a detectable change in one or more biological activities normally associated with the native polypeptide.

The term “activity” for the purposes herein refers to an action or effect of a component of a fusion protein consistent with that of the corresponding native biologically active protein, wherein “biological activity” refers to an in vitro or in vivo biological function or effect, including but not limited to receptor binding, antagonist activity, agonist activity, or a cellular or physiologic response.

As used herein. “treatment” or “treating.” “palliating.” and “ameliorating” are used interchangeably. These terms refer to an approach for obtaining beneficial or desired results including but not limited to a therapeutic benefit and/or a prophylactic benefit. By “therapeutic benefit” is meant eradication or amelioration of the underlying disorder being treated. Also, a therapeutic benefit is achieved with the cradication or amelioration of one or more of the physiological symptoms associated with the underlying disease condition such that an improvement is observed in the subject, notwithstanding that the subject may still be afflicted with the underlying disorder. For prophylactic benefit, the compositions may be administered to a subject at risk of developing a particular disease condition, or to a subject reporting one or more of the physiological symptoms of a disease, even though a diagnosis of this disease may not have been made.

The term “therapeutic effect” refers to a physiologic effect, including but not limited to the cure, mitigation, amelioration, or prevention of disease condition in humans or other animals, or to otherwise enhance physical or mental wellbeing of humans or animals, caused by a polypeptide of the present disclosure. Determination of a therapeutically effective amount is well within the capability of those skilled in the art, especially in light of the detailed disclosure provided herein.

The term “effective amount” refers to an amount of a biologically active protein, either alone or as a part of a fusion protein composition, that is capable of having any detectable, beneficial effect on any symptom, aspect, measured parameter or characteristics of a disease state or condition when administered in one or repeated doses to a subject. Such effect need not be absolute to be beneficial. The disease condition can refer to a disorder or a disease.

The term “pharmaceutically acceptable” refers to those compounds, materials, compositions, formulations or dosage forms which are, within the scope of sound medical judgment, suitable for use in contact with the tissues of human beings and other animals without excessive toxicity, irritation, allergic response, or other problem or complication, commensurate with a reasonable benefit/risk ratio.

The terms “formulation” and “dosage form” are used interchangeably and refer to a pharmaceutical composition that is formulated in accordance with clinical requirements, in a form that can be directly administered to a subject in need thereof for preventive or therapeutic use.

The term “pharmaceutically acceptable excipient” refers to a pharmaceutically acceptable material, carrier, or vehicle which are used for the preparation of the formulations or dosage forms in accordance with the present disclosure. Each excipient must be “acceptable” in the sense of being compatible with the other ingredients of the formulations or dosage forms and not injurious to the patient.

The term “subject” “individual” or “patient” as used herein refers to any animals that can be used in the present disclosure, including but not limited to human, primate, rodent, canine, feline, equine, ovine, porcine, and the like.

The terms “administer”. “administered”. “administers”. “administering” and “dosing” are used interchangeably and are defined as providing a compound, a composition, a formulation and/or a dosage form in accordance with the present disclosure to a subject in need thereof via a route known in the art, including but not limited to oral, buccal, topical, transmucosal, transdermal, rectal, and parenteral routes of administration. In some embodiments, an oral route of administration is used. In some embodiments, a parenteral route of administration, including intravenous, intraarterial, intramuscular, subcutaneous, intraosscous, and intraperitoneal is used. In some embodiments, a topical route of administration is used.

The term “in vivo” as used herein refers to an event that takes place in a subject's body.

The term “in vitro” as used herein refers to an event that takes places outside of a subject's body. In some embodiments, an in vitro assay encompasses any assay run outside of a subject assay. In vitro assays encompass cell-based assays in which cells alive or dead are employed. In vitro assays also encompass a cell-free assay in which no intact cells are employed.

The term “pain” as used herein refers to an unpleasant sensory and emotional experience associated with, or resembling that associated with, actual or potential tissue damage. In the context of the present disclosure, it is intended that the term “pain” comprises all types of acute pain, chronic pain (e.g., nociceptive pain, neuropathic pain and mixed pain) and transitional pain.

The terms “neuropathic pain” and “neuralgia” are used interchangeably herein and refer to pain caused by lesions or diseases of the bodies' somatosensory nervous system. The term “neuropathic pain” may be further divided into central neuropathic pain and peripheral neuropathic pain according to the locations of the lesions or diseases. In some embodiments, the neuropathic pain is peripheral neuropathic pain. In the context of the present disclosure, peripheral neuropathic pains of particular interest include, but not limited to peripheral neuropathic pain associated with metabolic disorder or ischemia, such as diabetic peripheral neuropathic pain or ischemic peripheral neuropathic pain: peripheral neuropathic pain associated with viral infection, such as post-herpetic neuralgia; and peripheral neuropathic pain associated with nerve or nerve root compression, such as sciatica.

The term “mixed pain” as used herein refers to a pain condition having the characteristics of nociceptive pain and neuropathic pain. Examples of mixed pain include, but not limited to arthritis pain, back pain, cancer pain, toothache, fibromyalgia, chronic inflammatory pain, lumbago, cervicodynia and eye pain. In the context of the present disclosure, mixed pains of particular interest include, but not limited to gout pain, cancer pain and eye pain. Gout is a common and complicated form of arthritis which is characterized by sudden (often at night), intense attacks of pain (most often in the big toc), in addition to swelling, redness and tenderness in one or more joints. Cancer pain may arise from a tumor compressing or infiltrating nearby body parts (i.e., a tumor-related cancer pain), from diagnostic and/or therapeutic procedures, or from skin, bonc, nerve and other changes caused by a hormone imbalance, an infection or an immune response. In some embodiments, cancer pain is cancer pain associated with tumor, such as nerve pain associated with cancer, bone pain associated with cancer, soft tissue pain associated with cancer and referred pain associated with cancer. In some embodiments, cancer pain is cancer pain associated with diagnostic and/or therapeutic procedures, such as phantom pain associated with cancer and cancer pain associated with chemotherapy. In some embodiments, the cancer pain associated with chemotherapy is cancer pain associated with chemotherapy-induced peripheral neuropathy (CIPN). In some embodiments, the chemotherapy is performed by administration of a cytotoxic agent to a subject in need thereof. In some embodiments, cancer pain is bone pain associated with cancer, such as bone pain associated with bone cancers (e.g., chondrosarcoma. Ewing's sarcoma, malignant fibrous histiocytoma of bone, osteosarcoma and fibrocartilaginous mesenchymoma of bone) and bone pain associated with bone metastatic tumors. In some embodiments, cancer pain is cancer pain associated with chemotherapy-induced peripheral neuropathy, such as cancer pain associated with platinum anticancer agents-induced peripheral neuropathy. Eye pain may be described as a sharp, aching or throbbing in one eye or both eyes. In some embodiments, the eye pain is at least one selected from the group consisting of ocular nociceptive pain and ocular neuropathic pain. Ocular nociceptive pain is often caused by the various insults present at the front of the eye, such as injury, surgery, contact lenses and foreign bodies. In contrast, ocular neuropathic pain refers to the heightened perception of eye pain in response to normally non-painful stimuli, and is often caused by factors such as allergies, infection, inflammation, chronic ocular surface disease, surgery, toxic keratopathy, radiation, ultraviolet light exposure, systemic neuropathy, trauma, trigeminal neuralgia and fibromyalgia. Ocular neuropathic pain may occur at different sites of eye area, including but not limited to cornea, conjunctiva, optic nerve, extraocular muscle, orbit and eyelid. Accordingly, the ocular neuropathic pain may be in the form of corneal neuralgia, conjunctival neuralgia, optic nerve neuralgia, extraocular muscle neuralgia, orbital neuralgia or cyclid neuralgia.

The terms “cancer” and “malignant tumors” are used interchangeably herein, and refer to a group of hyperproliferative diseases characterized by uncontrolled abnormal cell growth, with the potential to invade or spread to other parts of the body. In the context of the present disclosure, the term “cancer” comprises all types of malignant tumors, including carcinomas (i.e., cancers derived from epithelial cells), lymphomas and leukemias (i.e, cancers arising from hematopoietic cells which leave the marrow and tend to mature in the lymph nodes and blood, respectively), sarcomas (i.e., cancers arising from connective tissue), blastomas (i.e., cancers derived from immature precursor cells or embryonic tissue) and germ cell tumors (i.e., cancers derived from pluripotent cells). Examples of cancers include, but not limited to cancers of bone and muscle (e.g., chondrosarcoma. Ewing's sarcoma, malignant fibrous histiocytoma of bone, osteosarcoma, rhabdomyosarcoma, leiomyosarcoma, myxosarcoma and fibrocartilaginous mesenchymoma of bone), cancers of brain and nervous system (e.g., astrocytoma, brainstem glioma, pilocytic astrocytoma, cpendymoma, primitive neuroectodermal tumor, cerebellar astrocytoma, cerebral astrocytoma, glioblastoma, glioma, medulloblastoma, neuroblastoma, oligodendroglioma, pincal astrocytoma, pituitary adenoma, visual pathway and hypothalamic glioma), cancers of breast (e.g., inflammatory breast cancer, invasive lobular carcinoma, tubular carcinoma, invasive cribriform carcinoma, medullary carcinoma, phyllodes tumor, mammary secretory carcinoma and papillary carcinomas of the breast), cancers of endocrine system (e.g., adrenocortical carcinoma, islet cell carcinoma, multiple endocrine neoplasia syndrome, parathyroid cancer, pheochromocytoma, thyroid cancer and merkel cell carcinoma), cancers of eye (e.g., uveal melanoma, retinoblastoma and optic nerve glioma), gastrointestinal cancers (e.g., anal cancer, appendix cancer, cholangiocarcinoma, gastrointestinal carcinoid tumor, colon cancer, extrahepatic bile duct cancer, gallbladder cancer, gastric (stomach) cancer, gastrointestinal stromal tumor, hepatocellular cancer, pancreatic cancer, rectal cancer and small intestine cancer), genitourinary and gynecologic cancers (e.g., bladder cancer, cervical cancer, endometrial cancer, extragonadal germ cell tumor, ovarian cancer, ovarian germ cell tumor, penile cancer, kidney cancer, renal cell carcinoma, transitional cell cancer, prostate cancer, testicular cancer, gestational trophoblastic tumor, urethral cancer, uterine sarcoma, vaginal cancer, vulvar cancer and nephroblastoma), cancers of head and neck (e.g., esophageal cancer, head and neck cancer, nasopharyngeal carcinoma, oral cancer, oropharyngeal cancer, paranasal sinus and nasal cavity cancer, pharyngeal cancer, salivary gland cancer and hypopharyngeal cancer), hematopoictic cancers (e.g., leukemias such as acute biphenotypic leukemia, acute cosinophilic leukemia, acute lymphoblastic leukemia, acute myeloid leukemia, acute myeloid dendritic cell leukemia. B-cell prolymphocytic leukemia, chronic lymphocytic leukemia, chronic myelogenous leukemia, hairy cell leukemia, large granular lymphocytic leukemia, mast cell leukemia, precursor B lymphoblastic leukemia and T-cell prolymphocytic leukemia, lymphomas such as AIDS-related lymphoma, anaplastic large cell lymphoma, angioimmunoblastic T-cell lymphoma. Burkitt's lymphoma, cutaneous T-cell lymphoma, diffuse large B-cell lymphoma, follicular lymphoma, hepatosplenic T-cell lymphoma. Hodgkin's lymphoma, intravascular large B-cell lymphoma, lymphoplasmacytic lymphoma, lymphomatoid granulomatosis, mantle cell lymphoma, marginal zone B-cell lymphoma, mediastinal large B-cell lymphoma, mucosa-associated lymphoid tissue lymphoma, mycosis fungoides, nodal marginal zone B-cell lymphoma, non-hodgkin lymphoma, primary central nervous system lymphoma, primary cutaneous follicular lymphoma, primary cutaneous immunocytoma, primary effusion lymphoma, plasmablastic lymphoma. Sézary syndrome and splenic marginal zone lymphoma, as well as multiple myeloma and myelodysplastic syndromes), cancers of skin (e.g., basal cell carcinoma, squamous cell carcinoma, squamous cell skin cancer, skin adnexal tumors, melanoma. Merkel cell carcinoma, keratoacanthoma and sarcomas of primary cutaneous origin), thoracic and respiratory cancers (e.g., bronchial adenomas, mesothelioma, pleuropulmonary blastoma, laryngeal cancer, thymic carcinoma, thymoma, and lung cancers such as adenocarcinoma of the lung, small cell lung cancer, non-small cell lung cancer, squamous-cell carcinoma of the lung). Kaposi sarcoma, liposarcoma, desmoplastic small round cell tumor and epithelioid hemangioendothelioma. In the context of the present disclosure, it is intended that the term “cancer” covers both primary cancers and the metastatic tumors derived therefrom (e.g., a bone metastatic tumor). In addition, it is also intended that the term “cancer” covers early cancer, intermediate stage cancer and advanced cancer, including any metastatic, unresectable, recurrent or incurable cancers.

The term “cytotoxic agents” as used herein refers to substances that kill cells, particularly cancer cells. These agents may stop cancer cells from dividing and growing and may cause tumors to shrink in size.

The term “associated with” as used herein refers to “caused by”. For example. “pain associated with acute injuries” means pain caused by acute injuries, i.e., pain in which acute injuries at least play a role.

### Analgesic Polypeptide

In one aspect, provided is an artificial polypeptide of formula X-Y (VI).

In some embodiments. X of formula (VI) comprises a sequence having at least 70% identity with SEQ ID NO: 1. In some embodiments. X of formula (VI) comprises a sequence having at least 75% identity with SEQ ID NO: 1. In some embodiments. X of formula (VI) comprises a sequence having at least 80% identity with SEQ ID NO: 1. In some embodiments. X of formula (VI) comprises a sequence having at least 85% identity with SEQ ID NO: 1. In some embodiments. X of formula (VI) comprises a sequence having at least 90% identity with SEQ ID NO: 1. In some embodiments. X of formula (VI) comprises a sequence having at least 95% identity with SEQ ID NO: 1. In some embodiments. X of formula (VI) comprises a sequence having at most 95% identity with SEQ ID NO: 1. In some embodiments. X of formula (VI) comprises a sequence having at most 90% identity with SEQ ID NO: 1. In some embodiments. X of formula (VI) comprises a sequence having at most 85% identity with SEQ ID NO: 1. In some embodiments. X of formula (VI) comprises a sequence having at most 80% identity with SEQ ID NO: 1. In some embodiments. X of formula (VI) comprises a sequence having at most 75% identity with SEQ ID NO: 1.

In some embodiments. Y of formula (VI) is a moiety comprising a sequence having at least 70% identity with SEQ ID NO: 2. In some embodiments. Y of formula (VI) is a moiety comprising a sequence having at least 75% identity with SEQ ID NO: 2. In some embodiments. Y of formula (VI) is a moiety comprising a sequence having at least 80% identity with SEQ ID NO: 2. In some embodiments. Y of formula (VI) is a moiety comprising a sequence having at least 85% identity with SEQ ID NO: 2. In some embodiments. Y of formula (VI) is a moiety comprising a sequence having at least 90% identity with SEQ ID NO: 2. In some embodiments. Y of formula (VI) is a moiety comprising a sequence having at least 95% identity with SEQ ID NO: 2. In some embodiments. Y of formula (VI) comprises a sequence having at most 95% identity with SEQ ID NO: 2. In some embodiments. Y of formula (VI) comprises a sequence having at most 90% identity with SEQ ID NO: 2. In some embodiments. Y of formula (VI) comprises a sequence having at most 85% identity with SEQ ID NO: 2. In some embodiments. Y of formula (VI) comprises a sequence having at most 80% identity with SEQ ID NO: 2. In some embodiments. Y of formula (VI) comprises a sequence having at most 75% identity with SEQ ID NO: 2.

In some embodiments. Y of formula (VI) is hydrophobic moiety, at least 50% amino acids of Y are selected from I, V, L, F, C, M, and A. In some embodiments. Y of formula (VI) is hydrophobic moiety, at least 55% amino acids of Y are selected from I, V, L, F, C, M, and A. In some embodiments. Y of formula (VI) is hydrophobic moiety, at least 60% amino acids of Y are selected from I, V, L, F, C, M, and A. In some embodiments. Y of formula (VI) is hydrophobic moiety, at least 65% amino acids of Y are selected from I, V, L, F, C, M, and A. In some embodiments. Y of formula (VI) is hydrophobic moiety, at least 70% amino acids of Y are selected from I, V, L, F, C, M, and A. In some embodiments. Y of formula (VI) is hydrophobic moiety, at least 75% amino acids of Y are selected from I, V, L, F, C, M, and A. In some embodiments. Y of formula (VI) is hydrophobic moiety, at least 80% amino acids of Y are selected from I, V, L, F, C, M, and A. In some embodiments. Y of formula (VI) is hydrophobic moiety, at least 85% amino acids of Y are selected from I, V, L, F, C, M, and A. In some embodiments. Y of formula (VI) is hydrophobic moiety, at least 90% amino acids of Y are selected from I, V, L, F, C, M, and A.

In some embodiments. X of formula (VI) is a moiety comprising 40 to 65 amino acids, and a total number of R, K, T, A, N, Q, D, E, S, and G in X is more than 30. In some embodiments. X of formula (VI) is a moiety comprising 40 to 65 amino acids, and a total number of R, K, T, A, N, Q, D, E, S, and G in X is 31, 32, 33, 34, 35, 36, 37, 38, 39 or 40. In some embodiments. X of formula (VI) is a moiety comprising 40 to 65 amino acids, and a total number of W, Y, F, M, L, I, and V in X is no more than 20. In some embodiments. X of formula (VI) is a moiety comprising 40 to 65 amino acids, and a total number of W, Y, F, M, L, I, and V in X is 19, 18, 17, 16, 15 or 14.

In some embodiments. Y of formula (VI) is a moiety comprising 10 to 50 amino acids, and a total number of R, K, T, A, N, Q, D, E, S, and G in Y is more than 10. In some embodiments. Y of formula (VI) is a moiety comprising 10 to 50 amino acids, and a total number of R, K, T, A, N, Q, D, E, S, and G in Y is 11, 12, 13, 14 or 15. In some embodiments. Y of formula (VI) is a moiety comprising 10 to 50 amino acids, and a total number of W, Y, F, M, L, I, and V in X is no more than 20. In some embodiments. Y of formula (VI) is a moiety comprising 10 to 50 amino acids, and a total number of W, Y, F, M, L, I, and V in Y is 19, 18, 17, 16, 15 or 14.

In some embodiments. X of formula (VI) comprises a sequence having at least 70% identity with any one selected from SEQ ID NOs: 80-83. In some embodiments. X of formula (VI) comprises a sequence having at least 75% identity with any one selected from SEQ ID NOs: 80-83. In some embodiments. X of formula (VI) comprises a sequence having at least 80% identity with any one selected from SEQ ID NOs: 80-83. In some embodiments. X of formula (VI) comprises a sequence having at least 85% identity with any one selected from SEQ ID NOs: 80-83. In some embodiments. X of formula (VI) comprises a sequence having at least 90% identity with any one selected from SEQ ID NOs: 80-83. In some embodiments. X of formula (VI) comprises a sequence having at least 95% identity with any one selected from SEQ ID NOs: 80-83. In some embodiments. X of formula (VI) comprises a sequence having at least 96%, at least 97%, at least 98%, or at least 99% identity with any one selected from SEQ ID NOs: 80-83. In some embodiments. X of formula (VI) comprises a sequence of any one selected from SEQ ID NOs: 80-83. In some embodiments. X of formula (VI) is a sequence of any one selected from SEQ ID NOs: 80-83.

In some embodiments. Y of formula (VI) comprises a sequence having at least 70% identity with SEQ ID NO: 3. In some embodiments. Y of formula (VI) comprises a sequence having at least 75% identity with SEQ ID NO: 3. In some embodiments. Y of formula (VI) comprises a sequence having at least 80% identity with SEQ ID NO: 3. In some embodiments. Y of formula (VI) comprises a sequence having at least 85% identity with SEQ ID NO: 3. In some embodiments. Y of formula (VI) comprises a sequence having at least 90% identity with SEQ ID NO: 3. In some embodiments. Y of formula (VI) comprises a sequence having at least 95% identity with SEQ ID NO: 3. In some embodiments. Y of formula (VI) comprises a sequence having at least 96%, at least 97%, at least 98%, or at least 99% identity with SEQ ID NO: 3. In some embodiments. Y of formula (VI) comprises a sequence of SEQ ID NO: 3. In some embodiments. Y of formula (VI) is a sequence of SEQ ID NO: 3.

In some embodiments, the artificial polypeptide of formula (VI) comprises a sequence having at least 70% identity with any one selected from SEQ ID NOs: 90-93. In some embodiments, the artificial polypeptide of formula (VI) comprises a sequence having at least 75% identity with any one selected from SEQ ID NOs: 90-93. In some embodiments, the artificial polypeptide of formula (VI) comprises a sequence having at least 80% identity with any one selected from SEQ ID NOs: 90-93. In some embodiments, the artificial polypeptide of formula (VI) comprises a sequence having at least 85% identity with any one selected from SEQ ID NOs: 90-93. In some embodiments, the artificial polypeptide of formula (VI) comprises a sequence having at least 90% identity with any one selected from SEQ ID NOs: 90-93. In some embodiments, the artificial polypeptide of formula (VI) comprises a sequence having at least 95% identity with any one selected from SEQ ID NOs: 90-93. In some embodiments, the artificial polypeptide of formula (VI) comprises a sequence having at least 96%, at least 97%, at least 98%, or at least 99% identity with any one selected from SEQ ID NOs: 90-93. In some embodiments, the artificial polypeptide of formula (VI) comprises a sequence that is any one selected -3 ( ) WSGR Docket No. 64982-711.301 from SEQ ID NOs: 90-93. In some embodiments, the artificial polypeptide of formula (VI) is a sequence that is any one selected from SEQ ID NOs: 90-93.

In one aspect, provided is an artificial polypeptide of formula X-Y (VII).

In some embodiments. X is a moiety comprising a mutant of the sequence SEQ ID NO: 1, characterized in that the mutant has at least 1, 2, 3, 4, or 5 amino acid insertions relative to SEQ ID NO: 1. In some embodiments. X is a moiety comprising a mutant of the sequence SEQ ID NO: 1, characterized in that the mutant has at least 1 amino acid insertion relative to SEQ ID NO: 1. In some embodiments. X is a moiety comprising a mutant of the sequence SEQ ID NO: 1, characterized in that the mutant has 1 amino acid insertion relative to SEQ ID NO: 1. In some embodiments. X is a moiety comprising a mutant of the sequence SEQ ID NO: 1, characterized in that the mutant has at least 2 amino acid insertions relative to SEQ ID NO: 1. In some embodiments. X is a moiety comprising a mutant of the sequence SEQ ID NO: 1, characterized in that the mutant has 2 amino acid insertions relative to SEQ ID NO: 1. In some embodiments. X is a moiety comprising a mutant of the sequence SEQ ID NO: 1, characterized in that the mutant has at least 3 amino acid insertions relative to SEQ ID NO: 1. In some embodiments. X is a moiety comprising a mutant of the sequence SEQ ID NO: 1, characterized in that the mutant has 3 amino acid insertions relative to SEQ ID NO: 1. In some embodiments. X is a moiety comprising a mutant of the sequence SEQ ID NO: 1, characterized in that the mutant has at least 4 amino acid insertions relative to SEQ ID NO: 1. In some embodiments. X is a moiety comprising a mutant of the sequence SEQ ID NO: 1, characterized in that the mutant has 4 amino acid insertions relative to SEQ ID NO: 1. In some embodiments. X is a moiety comprising a mutant of the sequence SEQ ID NO: 1, characterized in that the mutant has at least 5 amino acid insertions relative to SEQ ID NO: 1. In some embodiments. X is a moiety comprising a mutant of the sequence SEQ ID NO: 1, characterized in that the mutant has 5 amino acid insertions relative to SEQ ID NO: 1.

In some embodiments, at least 20% amino acids of X in the mutant are selected from R, K, N, D, Q, E, and H. In some embodiments, at least 25% amino acids of X in the mutant are selected from R, K, N, D, Q, E, and H. In some embodiments, at least 30% amino acids of X in the mutant are selected from R, K, N, D, Q, E, and H. In some embodiments, at least 35% amino acids of X in the mutant are selected from R, K, N, D, Q, E, and H. In some embodiments, at least 40% amino acids of X in the mutant are selected from R, K, N, D, Q, E, and H. In some embodiments, at least 45% amino acids of X in the mutant are selected from R, K, N, D, Q, E, and H. In some embodiments, at least 50% amino acids of X in the mutant are selected from R, K, N, D, Q, E, and H. In some embodiments, at most 55% amino acids of X in the mutant are selected from R, K, N, D, Q, E, and H. In some embodiments, at most 60% amino acids of X in the mutant are selected from R, K, N, D, Q, E, and H. In some embodiments, at most 65% amino acids of X in the mutant are selected from R, K, N, D, Q, E, and H. In some embodiments, at most 70% amino acids of X in the mutant are selected from R, K, N, D, Q, E, and H. In some embodiments, at most 75% amino acids of X in the mutant are selected from R, K, N, D, Q, E, and H. In some embodiments, at most 80% amino acids of X in the mutant are selected from R, K, N, D, Q, E, and H. In some embodiments, at most 85% amino acids of X in the mutant are selected from R, K, N, D, Q, E, and H. In some embodiments, at most 90% amino acids of X in the mutant are selected from R, K, N, D, Q, E, and H.

In some embodiments. Y is a moiety comprising 10 to 30 amino acids. In some embodiments. Y is a moiety comprising 10 amino acids. 11 amino acids. 12 amino acids. 13 amino acids. 14 amino acids. 15 amino acids. 16 amino acids. 17 amino acids. 18 amino acids. 19 amino acids. 20 amino acids. 21 amino acids. 22 amino acids. 23 amino acids. 24 amino acids. 25 amino acids. 26 amino acids. 27 amino acids. 28 amino acids. 29 amino acids, or 30 amino acids. In some embodiments. Y is a moiety comprising 11 to 29 amino acids. In some embodiments. Y is a moiety comprising 12 to 28 amino acids. In some embodiments. Y is a moiety comprising 13 to 27 amino acids. In some embodiments. Y is a moiety comprising 14 to 26 amino acids. In some embodiments. Y is a moiety comprising 15 to 25 amino acids. In some embodiments. Y comprises 15 to 25 amino acids. In some embodiments. Y comprises 18 to 25 amino acids. In some embodiments. Y comprises 20 to 25 amino acids.

In some embodiments. Y comprises a sequence having at least 15 continuous AAs of SEQ ID NO: 2. In some embodiments. Y comprises a sequence having 15 continuous AAs of SEQ ID NO: 2. In some embodiments. Y comprises a sequence having at least 16 continuous AAs of SEQ ID NO: 2. In some embodiments. Y comprises a sequence having 16 continuous AAs of SEQ ID NO: 2. In some embodiments. Y comprises a sequence having at least 17 continuous AAs of SEQ ID NO: 2. In some embodiments. Y comprises a sequence having 17 continuous AAs of SEQ ID NO: 2. In some embodiments. Y comprises a sequence having at least 18 continuous AAs of SEQ ID NO: 2. In some embodiments. Y comprises a sequence having 18 continuous AAs of SEQ ID NO: 2. In some embodiments. Y comprises a sequence having at least 19 continuous AAs of SEQ ID NO: 2. In some embodiments. Y comprises a sequence having 19 continuous AAs of SEQ ID NO: 2. In some embodiments. Y comprises a sequence having at least 20 continuous AAs of SEQ ID NO: 2. In some embodiments. Y comprises a sequence having 20 continuous AAs of SEQ ID NO: 2. In some embodiments. Y comprises a sequence having at least 21 continuous AAs of SEQ ID NO: 2. In some embodiments. Y comprises a sequence having 21 continuous AAs of SEQ ID NO: 2. In some embodiments. Y comprises a sequence having at least 22 continuous AAs of SEQ ID NO: 2. In some embodiments. Y comprises a sequence having 22 continuous AAs of SEQ ID NO: 2. In some embodiments. Y comprises a sequence having at least 23 continuous AAs of SEQ ID NO: 2. In some embodiments. Y comprises a sequence having 23 continuous AAs of SEQ ID NO: 2. In some embodiments. Y comprises a sequence having at least 24 continuous AAs of SEQ ID NO: 2. In some embodiments. Y comprises a sequence having 24 continuous AAs of SEQ ID NO: 2. In some embodiments. Y comprises a sequence having at least 25 continuous AAs of SEQ ID NO: 2. In some embodiments. Y comprises a sequence having 25 continuous AAs of SEQ ID NO: 2.

In some embodiments. Y is a moiety comprising a mutant of the sequence SEQ ID NO: 2, characterized in that the mutant has at least 1, 2, 3, 4, 5, 6, 7, 8, 9, 10, 11, 12, 13, 14, 15, 16, 17, 18, 19, or 20 amino acid truncations relative to SEQ ID NO: 2. In some embodiments, the at least 1, 2, 3, 4, 5, 6, 7, 8, 9, 10, 11, 12, 13, 14, 15, 16, 17, 18, 19, or 20 amino acid truncations are located at the C-terminal of Y. In some embodiments. Y is a moiety comprising a mutant of the sequence SEQ ID NO: 2, characterized in that the mutant has 1, 2, 3, 4, 5, 6, 7, 8, 9, 10, 11, 12, 13, 14, 15, 16, 17, 18, 19, or 20 amino acid truncations relative to SEQ ID NO: 2. In some embodiments, the 1, 2, 3, 4, 5, 6, 7, 8, 9, 10, 11, 12, 13, 14, 15, 16, 17, 18, 19, or 20 amino acid truncations are located at the C-terminal of Y.

In some embodiments, a total number of E or D in X is at least 8. In some embodiments, a total number of E or D in X is 8. In some embodiments, a total number of E or D in X is at least 9. In some embodiments, a total number of E or D in X is 9. In some embodiments, a total number of E or D in X is at least 10. In some embodiments, a total number of E or D in X is 10. In some embodiments, a total number of E or D in X is at least 11. In some embodiments, a total number of E or D in X is 11. In some embodiments, a total number of E or D in X is at least 12. In some embodiments, a total number of E or D in X is 12. In some embodiments, a total number of E or D in X is at most 15. In some embodiments, a total number of E or D in X is 15. In some embodiments, a total number of E or D in X is at most 14. In some embodiments, a total number of E or D in X is 14. In some embodiments, a total number of E or D in X is at most 13. In some embodiments, a total number of E or D in X is 13. In some embodiments, a total number of E or D in X is at most 12. In some embodiments, a total number of E or D in X is 12. In some embodiments, a total number of E or D in X is at most 11. In some embodiments, a total number of E or D in X is 11. In some embodiments, a total number of E or D in X is at most 10. In some embodiments, a total number of E or D in X is 10.

In some embodiments. X of formula (VII) is a moiety comprising 40 to 65 amino acids, and a total number of R, K, T, A, N, Q, D, E, S, and G in X is more than 30. In some embodiments. X of formula (VII) is a moiety comprising 40 to 65 amino acids, and a total number of R, K, T, A, N, Q, D, E, S, and G in X is 31, 32, 33, 34, 35, 36, 37, 38, 39 or 40. In some embodiments. X of formula (VII) is a moiety comprising 40 to 65 amino acids, and a total number of W, Y, F, M, L, I, and V in X is no more than 20. In some embodiments. X of formula (VII) is a moiety comprising 40 to 65 amino acids, and a total number of W, Y, F, M, L, I, and V in X is 19, 18, 17, 16, 15 or 14.

In some embodiments. 40-65% amino acids of Y are selected from I, V, L, F, C, M, and A. In some embodiments, at least 40% amino acids of Y are selected from I, V, L, F, C, M, and A. In some embodiments, at least 45% amino acids of Y are selected from I, V, L, F, C, M, and A. In some embodiments, at least 50% amino acids of Y are selected from I, V, L, F, C, M, and A. In some embodiments, at least 55% amino acids of Y are selected from I, V, L, F, C, M, and A. In some embodiments, at least 60% amino acids of Y are selected from I, V, L, F, C, M, and A. In some embodiments, at most 65% amino acids of Y are selected from I, V, L, F, C, M, and A. In some embodiments, at most 60% amino acids of Y are selected from I, V, L, F, C. M, and A. In some embodiments, at most 55% amino acids of Y are selected from I, V, L, F, C, M, and A. In some embodiments, at most 50% amino acids of Y are selected from I, V, L, F, C, M, and A. In some embodiments, at most 45% amino acids of Y are selected from I, V, L, F, C, M, and A.

In some embodiments, a total number of R, K, T, A, N, Q, D, E, S, and G in Y is no more than 10. In some embodiments, a total number of R, K, T, A, N, Q, D, E, S, and G in Y is 1, 2, 3, 4, 5, 6, 7, 8, 9 or 10. In some embodiments, a total number of W, Y, F, M, L, I, and V in Y is no more than 15. In some embodiments, a total number of W, Y, F, M, L, I, and V in Y is 5, 6, 7, 8, 9, 10, 11, 12, 13, 14 or 15.

In some embodiments. X of formula (VII) comprises a sequence having at least 70% identity with SEQ ID NO: 96. In some embodiments. X of formula (VII) comprises a sequence having at least 75% identity with SEQ ID NO: 96. In some embodiments. X of formula (VII) comprises a sequence having at least 80% identity with SEQ ID NO: 96. In some embodiments. X of formula (VII) comprises a sequence having at least 85% identity with SEQ ID NO: 96. In some embodiments. X of formula (VII) comprises a sequence having at least 90% identity with SEQ ID NO: 96. In some embodiments. X of formula (VII) comprises a sequence having at least 95% identity with SEQ ID NO: 96. In some embodiments. X of formula (VII) comprises a sequence having at least 96%, at least 97%, at least 98%, or at least 99% identity with SEQ ID NO: 96. In some embodiments. X of formula (VII) comprises a sequence of SEQ ID NO: 96. In some embodiments. X of formula (VII) is a sequence of SEQ ID NO: 96.

In some embodiments. Y of formula (VII) comprises a sequence having at least 70% identity with any one selected from SEQ ID NOs: 97-103. In some embodiments. Y of formula (VII) comprises a sequence having at least 75% identity with any one selected from SEQ ID NOs: 97-103. In some embodiments. Y of formula (VII) comprises a sequence having at least 80% identity with any one selected from SEQ ID NOs: 97-103. In some embodiments. Y of formula (VII) comprises a sequence having at least 85% identity with any one selected from SEQ ID NOs: 97-103. In some embodiments. Y of formula (VII) comprises a sequence having at least 90% identity with any one selected from SEQ ID NOs: 97-103. In some embodiments. Y of formula (VII) comprises a sequence having at least 95% identity with any one selected from SEQ ID NOs: 97-103. In some embodiments. Y of formula (VII) comprises a sequence having at least 96%, at least 97%, at least 98%, or at least 99% identity with any one selected from SEQ ID NOs: 97-103. In some embodiments. Y of formula (VII) comprises a sequence that is any one selected from SEQ ID NOs: 97-103. In some embodiments. Y of formula (VII) is a sequence that is any one selected from SEQ ID NOs: 97-103.

In some embodiments, the artificial polypeptide of formula (VII) comprises a sequence having at least 70% identity with any one selected from SEQ ID NOs: 104-110. In some embodiments, the artificial polypeptide of formula (VII) comprises a sequence having at least 75% identity with any one selected from SEQ ID NOs: 104-110. In some embodiments, the artificial polypeptide of formula (VII) comprises a sequence having at least 80% identity with any one selected from SEQ ID NOs: 104-110. In some embodiments, the artificial polypeptide of formula (VII) comprises a sequence having at least 85% identity with any one selected from SEQ ID NOs: 104-110. In some embodiments, the artificial polypeptide of formula (VII) comprises a sequence having at least 90% identity with any one selected from SEQ ID NOs: 104-110. In some embodiments, the artificial polypeptide of formula (VII) comprises a sequence having at least 95% identity with any one selected from SEQ ID NOs: 104-110. In some embodiments, the artificial polypeptide of formula (VII) comprises a sequence having at least 96%, at least 97%, at least 98%, or at least 99% identity with any one selected from SEQ ID NOs: 104-110. In some embodiments, the artificial polypeptide of formula (VII) comprises a sequence that is any one selected from SEQ ID NOs: 104-110. In some embodiments, the artificial polypeptide of formula (VII) is a sequence that is any one selected from SEQ ID NOs: 104-110. In another aspect, provided is a mutant of artificial polypeptide of formula (I).

In some embodiments, at least 20% amino acids of X in the mutant are selected from R, K, N, D, Q, E, and H. In some embodiments, at least 25% amino acids of X in the mutant are selected from R, K, N, D, Q, E, and H. In some embodiments, at least 30% amino acids of X in the mutant are selected from R, K, N, D, Q, E, and H. In some embodiments, at least 35% amino acids of X in the mutant are selected from R, K, N, D, Q, E, and H. In some embodiments, at least 40% amino acids of X in the mutant are selected from R, K, N, D, Q, E, and H. In some embodiments, at least 45% amino acids of X in the mutant are selected from R, K, N, D, Q, E, and H. In some embodiments, at least 50% amino acids of X in the mutant are selected from R, K, N, D, Q, E, and H. In some embodiments, at most 55% amino acids of X in the mutant are selected from R, K, N, D, Q, E, and H. In some embodiments, at most 60% amino acids of X in the mutant are selected from R, K, N, D, Q, E, and H. In some embodiments, at most 65% amino acids of X in the mutant are selected from R, K, N, D, Q, E, and H. In some embodiments, at most 70% amino acids of X in the mutant are selected from R, K, N, D, Q, E, and H. In some embodiments, at most 75% amino acids of X in the mutant are selected from R, K, N, D, Q, E, and H. In some embodiments, at most 80% amino acids of X in the mutant are selected from R, K, N, D, Q, E, and H. In some embodiments, at most 85% amino acids of X in the mutant are selected from R, K, N, D, Q, E, and H. In some embodiments, at most 90% amino acids of X in the mutant are selected from R, K, N, D, Q, E, and H.

In some embodiments. X in the mutant comprises a sequence having at least 70% identity with SEQ ID NO: 1. In some embodiments. X in the mutant comprises a sequence having at least 75% identity with SEQ ID NO: 1. In some embodiments. X in the mutant comprises a sequence having at least 80% identity with SEQ ID NO: 1. In some embodiments. X in the mutant comprises a sequence having at least 85% identity with SEQ ID NO: 1. In some embodiments. X in the mutant comprises a sequence having at least 90% identity with SEQ ID NO: 1. In some embodiments. X in the mutant comprises a sequence having at least 95% identity with SEQ ID NO: 1.

In some embodiments. Y in the mutant can be a moiety comprising 10 to 50 amino acids, and a total number of Cysteine (C) of Y is 5. In some embodiments. Y in the mutant can be a moiety comprising 10 to 50 amino acids, and a total number of Cysteine (C) of Y is less than 5. In some embodiments. Y in the mutant can be a moiety comprising 10 to 50 amino acids, and a total number of Cysteine (C) of Y is 4. In some embodiments. Y in the mutant can be a moiety comprising 10 to 50 amino acids, and a total number of Cysteine (C) of Y is less than 4. In some embodiments. Y in the mutant can be a moiety comprising 10 to 50 amino acids, and a total number of Cysteine (C) of Y is 3. In some embodiments. Y in the mutant can be a moiety comprising 10 to 50 amino acids, and a total number of Cysteine (C) of Y is less than 3. In some embodiments. Y in the mutant can be a moiety comprising 10 to 50 amino acids, and a total number of Cysteine (C) of Y is 2. In some embodiments. Y in the mutant can be a moiety comprising 10 to 50 amino acids, and a total number of Cysteine (C) of Y is less than 2. In some embodiments. Y in the mutant can be a moiety comprising 10 to 50 amino acids, and a total number of Cysteine (C) of Y is 1. In some embodiments. Y in the mutant can be a moiety comprising 10 to 50 amino acids, and a total number of Cysteine (C) of Y is less than 1. In some embodiments. Y in the mutant can be a moiety comprising 10 to 50 amino acids, and a total number of Cysteine (C) of Y is 0.

In some embodiments, a total number of hydrophobic amino acids in Y in the mutant is more than 8. In some embodiments, a total number of hydrophobic amino acids in Y in the mutant is more than 9. In some embodiments, a total number of hydrophobic amino acids in Y in the mutant is more than 10. In some embodiments, a total number of hydrophobic amino acids in Y in the mutant is more than 11. In some embodiments, the total number of hydrophobic amino acids in Y in the mutant is more than 12. In some embodiments, the total number of hydrophobic amino acids in Y in the mutant is more than 13. In some embodiments, the total number of hydrophobic amino acids in Y in the mutant is more than 14. In some embodiments, the total number of hydrophobic amino acids in Y in the mutant is more than 15.

In some embodiments, a total number of hydrophilic amino acids in Y in the mutant is 5. In some embodiments, a total number of hydrophilic amino acids in Y in the mutant is no more than 5. In some embodiments, a total number of hydrophilic amino acids in Y in the mutant is 4. In some embodiments, a total number of hydrophilic amino acids in Y in the mutant is no more than 4. In some embodiments, a total number of hydrophilic amino acids in Y in the mutant is 3. In some embodiments, a total number of hydrophilic amino acids in Y in the mutant is no more than 3. In some embodiments, a total number of hydrophilic amino acids in Y in the mutant is 2. In some embodiments, a total number of hydrophilic amino acids in Y in the mutant is no more than 2. In some embodiments, a total number of hydrophilic amino acids in Y in the mutant is 1. In some embodiments, a total number of hydrophilic amino acids in Y in the mutant is no more than 1.

In some embodiments. Y in the mutant comprises a sequence having at least 70% identity with any one selected from SEQ ID NOs: 3-15, 58-61 and 84-85. In some embodiments. Y in the mutant comprises a sequence having at least 75% identity with any one selected from SEQ ID NOs: 3-15, 58-61 and 84-85. In some embodiments. Y in the mutant comprises a sequence having at least 80% identity with any one selected from SEQ ID NOs: 3-15, 58-61 and 84-85. In some embodiments. Y in the mutant comprises a sequence having at least 85% identity with any one selected from SEQ ID NOs: 3-15, 58-61 and 84-85. In some embodiments. Y in the mutant comprises a sequence having at least 90% identity with any one selected from SEQ ID NOs: 3-15, 58-61 and 84-85. In some embodiments. Y in the mutant comprises a sequence having at least 95% identity with any one selected from SEQ ID NOs: 3-15, 58-61 and 84-85. In some embodiments. Y in the mutant comprises a sequence having at least 96%, at least 97%, at least 98%, or at least 99% identity with any one selected from SEQ ID NOs: 3-15, 58-61 and 84-85. In some embodiments. Y in the mutant comprises a sequence of any one selected from SEQ ID NOs: 3-15, 58-61 and 84-85. In some embodiments. Y in the mutant is a sequence of any one selected from SEQ ID NOs: 3-15, 58-61 and 84-85.

In some embodiments. Y in the mutant comprises a sequence having at least 70% identity with any one selected from SEQ ID NOs: 3-18, 58-61 and 84-85. In some embodiments. Y in the mutant comprises a sequence having at least 75% identity with any one selected from SEQ ID NOs: 3-18, 58-61 and 84-85. In some embodiments. Y in the mutant comprises a sequence having at least 80% identity with any one selected from SEQ ID NOs: 3-18, 58-61 and 84-85. In some embodiments. Y in the mutant comprises a sequence having at least 85% identity with any one selected from SEQ ID NOs: 3-18, 58-61 and 84-85. In some embodiments. Y in the mutant comprises a sequence having at least 90% identity with any one selected from SEQ ID NOs: 3-18, 58-61 and 84-85. In some embodiments. Y in the mutant comprises a sequence having at least 95% identity with any one selected from SEQ ID NOs: 3-18, 58-61 and 84-85. In some embodiments. Y in the mutant comprises a sequence having at least 96%, at least 97%, at least 98%, or at least 99% identity with any one selected from SEQ ID NOs: 3-18, 58-61 and 84-85. In some embodiments. Y in the mutant comprises a sequence of any one selected from SEQ ID NOs: 3-18, 58-61 and 84-85. In some embodiments. Y in the mutant is a sequence of any one selected from SEQ ID NOs: 3-18, 58-61 and 84-85.

In another aspect, provided is a mutant of artificial polypeptide of formula (II).

In some embodiments, at least 20% amino acids of X in the mutant are selected from R, K, N, D, Q, E, and H. In some embodiments, at least 25% amino acids of X in the mutant are selected from R, K, N, D, Q, E, and H. In some embodiments, at least 30% amino acids of X in the mutant are selected from R, K, N, D, Q, E, and H. In some embodiments, at least 35% amino acids of X in the mutant are selected from R, K, N, D, Q, E, and H. In some embodiments, at least 40% amino acids of X in the mutant are selected from R, K, N, D, Q, E, and H. In some embodiments, at least 45% amino acids of X in the mutant are selected from R, K, N, D, Q, E, and H. In some embodiments, at least 50% amino acids of X in the mutant are selected from R, K, N, D, Q, E, and H. In some embodiments, at most 55% amino acids of X in the mutant are selected from R, K, N, D, Q, E, and H. In some embodiments, at most 60% amino acids of X in the mutant are selected from R, K, N, D, Q, E, and H. In some embodiments, at most 65% amino acids of X in the mutant are selected from R, K, N, D, Q, E, and H. In some embodiments, at most 70% amino acids of X in the mutant are selected from R, K, N, D, Q, E, and H. In some embodiments, at most 75% amino acids of X in the mutant are selected from R, K, N, D, Q, E, and H. In some embodiments, at most 80% amino acids of X in the mutant are selected from R, K, N, D, Q, E, and H. In some embodiments, at most 85% amino acids of X in the mutant are selected from R, K, N, D, Q, E, and H. In some embodiments, at most 90% amino acids of X in the mutant are selected from R, K, N, D, Q, E, and H.

In some embodiments. X in the mutant comprises a sequence having at least 70% identity with SEQ ID NO: 1. In some embodiments. X in the mutant comprises a sequence having at least 75% identity with SEQ ID NO: 1. In some embodiments. X in the mutant comprises a sequence having at least 80% identity with SEQ ID NO: 1. In some embodiments. X in the mutant comprises a sequence having at least 85% identity with SEQ ID NO: 1. In some embodiments. X in the mutant comprises a sequence having at least 90% identity with SEQ ID NO: 1. In some embodiments. X in the mutant comprises a sequence having at least 95% identity with SEQ ID NO: 1.

In some embodiments. Y in the mutant comprises a sequence having at most 50% identity with SEQ ID NO: 2. In some embodiments. Y in the mutant comprises a sequence having at most 55% identity with SEQ ID NO: 2. In some embodiments. Y in the mutant comprises a sequence having at most 60% identity with SEQ ID NO: 2. In some embodiments. Y in the mutant comprises a sequence having at most 65% identity with SEQ ID NO: 2. In some embodiments. Y in the mutant comprises a sequence having at most 70% identity with SEQ ID NO: 2. In some embodiments. Y in the mutant comprises a sequence having at most 75% identity with SEQ ID NO: 2. In some embodiments. Y in the mutant comprises a sequence having at most 80% identity with SEQ ID NO: 2. In some embodiments. Y in the mutant comprises a sequence having at most 85% identity with SEQ ID NO: 2. In some embodiments. Y in the mutant comprises a sequence having at most 90% identity with SEQ ID NO: 2.

In some embodiments, a total number of hydrophobic amino acids in Y in the mutant is more than 8. In some embodiments, a total number of hydrophobic amino acids in Y in the mutant is more than 9. In some embodiments, a total number of hydrophobic amino acids in Y in the mutant is more than 10. In some embodiments, a total number of hydrophobic amino acids in Y in the mutant is more than 11. In some embodiments, the total number of hydrophobic amino acids in Y in the mutant is more than 12. In some embodiments, the total number of hydrophobic amino acids in Y in the mutant is more than 13. In some embodiments, the total number of hydrophobic amino acids in Y in the mutant is more than 14. In some embodiments, the total number of hydrophobic amino acids in Y in the mutant is more than 15.

In some embodiments, a total number of hydrophilic amino acids in Y in the mutant is 5. In some embodiments, a total number of hydrophilic amino acids in Y in the mutant is no more than 5. In some embodiments, a total number of hydrophilic amino acids in Y in the mutant is 4. In some embodiments, a total number of hydrophilic amino acids in Y in the mutant is no more than 4. In some embodiments, a total number of hydrophilic amino acids in Y in the mutant is 3. In some embodiments, a total number of hydrophilic amino acids in Y in the mutant is no more than 3. In some embodiments, a total number of hydrophilic amino acids in Y in the mutant is 2. In some embodiments, a total number of hydrophilic amino acids in Y in the mutant is no more than 2. In some embodiments, a total number of hydrophilic amino acids in Y in the mutant is 1. In some embodiments, a total number of hydrophilic amino acids in Y in the mutant is no more than 1.

In some embodiments. Y in the mutant comprises a sequence having at least 70% identity with any one selected from SEQ ID NOs: 16-18. In some embodiments. Y in the mutant comprises a sequence having at least 75% identity with any one selected from SEQ ID NOs: 16-18. In some embodiments. Y in the mutant comprises a sequence having at least 80% identity with any one selected from SEQ ID NOs: 16-18. In some embodiments. Y in the mutant comprises a sequence having at least 85% identity with any one selected from SEQ ID NOs: 16-18. In some embodiments. Y in the mutant comprises a sequence having at least 90% identity with any one selected from SEQ ID NOs: 16-18. In some embodiments. Y in the mutant comprises a sequence having at least 95% identity with any one selected from SEQ ID NOs: 16-18. In some embodiments. Y in the mutant comprises a sequence having at least 96%, at least 97%, at least 98%, or at least 99% identity with any one selected from SEQ ID NOs: 16-18. In some embodiments. Y in the mutant comprises a sequence of any one selected from SEQ ID NOs: 16-18. In some embodiments. Y in the mutant is a sequence of any one selected from SEQ ID NOs: 16-18.

In another aspect, provided is a mutant of artificial polypeptide of formula (III).

In some embodiments, at least 20% amino acids of X in the mutant are selected from R, K, N, D, Q, E, and H. In some embodiments, at least 25% amino acids of X in the mutant are selected from R, K, N, D, Q, E, and H. In some embodiments, at least 30% amino acids of X in the mutant are selected from R, K, N, D, Q, E, and H. In some embodiments, at least 35% amino acids of X in the mutant are selected from R, K, N, D, Q, E, and H. In some embodiments, at least 40% amino acids of X in the mutant are selected from R, K, N, D, Q, E, and H. In some embodiments, at least 45% amino acids of X in the mutant are selected from R, K, N, D, Q, E, and H. In some embodiments, at least 50% amino acids of X in the mutant are selected from R, K, N, D, Q, E, and H. In some embodiments, at most 55% amino acids of X in the mutant are selected from R, K, N, D, Q, E, and H. In some embodiments, at most 60% amino acids of X in the mutant are selected from R, K, N, D, Q, E, and H. In some embodiments, at most 65% amino acids of X in the mutant are selected from R, K, N, D, Q, E, and H. In some embodiments, at most 70% amino acids of X in the mutant are selected from R, K, N, D, Q, E, and H. In some embodiments, at most 75% amino acids of X in the mutant are selected from R, K, N, D, Q, E, and H. In some embodiments, at most 80% amino acids of X in the mutant are selected from R, K, N, D, Q, E, and H. In some embodiments, at most 85% amino acids of X in the mutant are selected from R, K, N, D, Q, E, and H. In some embodiments, at most 90% amino acids of X in the mutant are selected from R, K, N, D, Q, E, and H.

In some embodiments. X in the mutant is a moiety comprising 40 to 65 amino acids, and a total number of H, R, K, D, Q, N and E in X is less than 33. In some embodiments. X in the mutant is a moiety comprising 40 to 65 amino acids, and a total number of H, R, K, D, Q, N and E in X is less than 32. In some embodiments. X in the mutant is a moiety comprising 40 to 65 amino acids, and a total number of H, R, K, D, Q, N and E in X is less than 31. In some embodiments. X in the mutant is a moiety comprising 40 to 65 amino acids, and a total number of H, R, K, D, Q, N and E in X is less than 30. In some embodiments. X in the mutant is a moiety comprising 40 to 65 amino acids, and a total number of H, R, K, D, Q, N and E in X is less than 29. In some embodiments. X in the mutant is a moiety comprising 40 to 65 amino acids, and a total number of H, R, K, D, Q, N and E in X is less than 28. In some embodiments. X in the mutant is a moiety comprising 40 to 65 amino acids, and a total number of H, R, K, D, Q, N and E in X is less than 27. In some embodiments. X in the mutant is a moiety comprising 40 to 65 amino acids, and a total number of H, R, K, D, Q, N and E in X is less than 26. In some embodiments. X in the mutant is a moiety comprising 40 to 65 amino acids, and a total number of H, R, K, D, Q, N and E in X is less than 25. In some embodiments. X in the mutant is a moiety comprising 40 to 65 amino acids, and a total number of H, R, K, D, Q, N and E in X is less than 24. In some embodiments. X in the mutant is a moiety comprising 40 to 65 amino acids, and a total number of H, R, K, D, Q, N and E in X is less than 23. In some embodiments. X in the mutant is a moiety comprising 40 to 65 amino acids, and a total number of H, R, K, D, Q, N and E in X is less than 22. In some embodiments. X in the mutant is a moiety comprising 40 to 65 amino acids, and a total number of H, R, K, D, Q, N and E in X is less than 21. In some embodiments. X in the mutant is a moiety comprising 40 to 65 amino acids, and a total number of H, R, K, D, Q, N and E in X is less than 20.

In some embodiments. X in the mutant is a moiety comprising 40 to 65 amino acids, and a total number of hydrophilic amino acids in X is more than 10. In some embodiments. X in the mutant is a moiety comprising 40 to 65 amino acids, and a total number of hydrophilic amino acids in X is more than 11. In some embodiments. X in the mutant is a moiety comprising 40 to 65 amino acids, and a total number of hydrophilic amino acids in X is more than 12. In some embodiments. X in the mutant is a moiety comprising 40 to 65 amino acids, and a total number of hydrophilic amino acids in X is more than 13. In some embodiments. X in the mutant is a moiety comprising 40 to 65 amino acids, and a total number of hydrophilic amino acids in X is more than 14. In some embodiments. X in the mutant is a moiety comprising 40 to 65 amino acids, and a total number of hydrophilic amino acids in X is more than 15. In some embodiments. X in the mutant is a moiety comprising 40 to 65 amino acids, and a total number of hydrophilic amino acids in X is more than 16. In some embodiments. X in the mutant is a moiety comprising 40 to 65 amino acids, and a total number of hydrophilic amino acids in X is more than 17. In some embodiments. X in the mutant is a moiety comprising 40 to 65 amino acids, and a total number of hydrophilic amino acids in X is more than 18. In some embodiments. X in the mutant is a moiety comprising 40 to 65 amino acids, and a total number of hydrophilic amino acids in X is more than 19. In some embodiments. X in the mutant is a moiety comprising 40 to 65 amino acids, and a total number of hydrophilic amino acids in X is more than 20. In some embodiments. X in the mutant is a moiety comprising 40 to 65 amino acids, and a total number of hydrophilic amino acids in X is more than 21. In some embodiments. X in the mutant is a moiety comprising 40 to 65 amino acids, and a total number of hydrophilic amino acids in X is more than 22. In some embodiments. X in the mutant is a moiety comprising 40 to 65 amino acids, and a total number of hydrophilic amino acids in X is more than 23. In some embodiments. X in the mutant is a moiety comprising 40 to 65 amino acids, and a total number of hydrophilic amino acids in X is more than 24. In some embodiments. X in the mutant is a moiety comprising 40 to 65 amino acids, and a total number of hydrophilic amino acids in X is more than 25.

In some embodiments. X in the mutant is a moiety comprising 40 to 65 amino acids, and a total number of hydrophobic amino acids in X is no more than 15. In some embodiments. X in the mutant is a moiety comprising 40 to 65 amino acids, and a total number of hydrophobic amino acids in X is no more than 14. In some embodiments. X in the mutant is a moiety comprising 40 to 65 amino acids, and a total number of hydrophobic amino acids in X is no more than 13. In some embodiments. X in the mutant is a moiety comprising 40 to 65 amino acids, and a total number of hydrophobic amino acids in X is no more than 12. In some embodiments. X in the mutant is a moiety comprising 40 to 65 amino acids, and a total number of hydrophobic amino acids in X is no more than 11. In some embodiments. X in the mutant is a moiety comprising 40 to 65 amino acids, and a total number of hydrophobic amino acids in X is no more than 10. In some embodiments. X in the mutant is a moiety comprising 40 to 65 amino acids, and a total number of hydrophobic amino acids in X is no more than 5.

In some embodiments. X in the mutant comprises a sequence having at least 70% identity with SEQ ID NO: 1. In some embodiments. X in the mutant comprises a sequence having at least 75% identity with SEQ ID NO: 1. In some embodiments. X in the mutant comprises a sequence having at least 80% identity with SEQ ID NO: 1. In some embodiments. X in the mutant comprises a sequence having at least 85% identity with SEQ ID NO: 1. In some embodiments. X in the mutant comprises a sequence having at least 90% identity with SEQ ID NO: 1. In some embodiments. X in the mutant comprises a sequence having at least 95% identity with SEQ ID NO: 1.

In some embodiments. Y in the mutant is a moiety comprising a sequence having at least 70% identity with SEQ ID NO: 2. In some embodiments. Y in the mutant is a moiety comprising a sequence having at least 75% identity with SEQ ID NO: 2. In some embodiments. Y in the mutant is a moiety comprising a sequence having at least 80% identity with SEQ ID NO: 2. In some embodiments. Y in the mutant is a moiety comprising a sequence having at least 85% identity with SEQ ID NO: 2. In some embodiments. Y in the mutant is a moiety comprising a sequence having at least 90% identity with SEQ ID NO: 2. In some embodiments. Y in the mutant is a moiety comprising a sequence having at least 95% identity with SEQ ID NO: 2. In some embodiments. Y in the mutant is a moiety comprising a sequence having at least 96%, at least 97%, at least 98%, or at least 99% identity with SEQ ID NO: 2. In some embodiments. Y in the mutant is a moiety comprising a sequence of SEQ ID NO: 2. In some embodiments. Y in the mutant is a moiety that is a sequence of SEQ ID NO: 2.

In some embodiments. Y in the mutant is hydrophobic moiety, at least 50% amino acids of Y are selected from I, V, L, F, C, M, and A. In some embodiments. Y in the mutant is hydrophobic moiety, at least 55% amino acids of Y are selected from I, V, L, F, C, M, and A. In some embodiments. Y in the mutant is hydrophobic moiety, at least 60% amino acids of Y are selected from I, V, L, F, C, M, and A. In some embodiments. Y in the mutant is hydrophobic moiety, at least 65% amino acids of Y are selected from I. V, L, F, C, M, and A. In some embodiments. Y in the mutant is hydrophobic moiety, at least 70% amino acids of Y are selected from I, V, L, F, C, M, and A. In some embodiments. Y in the mutant is hydrophobic moiety, at least 75% amino acids of Y are selected from I, V, L, F, C, M, and A. In some embodiments. Y in the mutant is hydrophobic moiety, at least 80% amino acids of Y are selected from I, V, L, F, C, M, and A. In some embodiments. Y in the mutant is hydrophobic moiety, at least 85% amino acids of Y are selected from I. V, L, F, C, M, and A. In some embodiments. Y in the mutant is hydrophobic moiety, at least 90% amino acids of Y are selected from I, V, L, F, C, M, and A.

In another aspect, provided is a mutant of artificial polypeptide of formula (IV).

In some embodiments, at least 20% amino acids of X in the mutant are selected from R, K, N, D, Q, E, and H. In some embodiments, at least 25% amino acids of X in the mutant are selected from R, K, N, D, Q, E, and H. In some embodiments, at least 30% amino acids of X in the mutant are selected from R, K, N, D, Q, E, and H. In some embodiments, at least 35% amino acids of X in the mutant are selected from R, K, N, D, Q, E, and H. In some embodiments, at least 40% amino acids of X in the mutant are selected from R, K, N, D, Q, E, and H. In some embodiments, at least 45% amino acids of X in the mutant are selected from R, K, N, D, Q, E, and H. In some embodiments, at least 50% amino acids of X in the mutant are selected from R, K, N, D, Q, E, and H. In some embodiments, at most 55% amino acids of X in the mutant are selected from R, K, N, D, Q, E, and H. In some embodiments, at most 60% amino acids of X in the mutant are selected from R, K, N, D, Q, E, and H. In some embodiments, at most 65% amino acids of X in the mutant are selected from R, K, N, D, Q, E, and H. In some embodiments, at most 70% amino acids of X in the mutant are selected from R, K, N, D, Q, E, and H. In some embodiments, at most 75% amino acids of X in the mutant are selected from R, K, N, D, Q, E, and H. In some embodiments, at most 80% amino acids of X in the mutant are selected from R, K, N, D, Q, E, and H. In some embodiments, at most 85% amino acids of X in the mutant are selected from R, K, N, D, Q, E, and H. In some embodiments, at most 90% amino acids of X in the mutant are selected from R, K, N, D, Q, E, and H.

In some embodiments. X in the mutant comprises 40 to 65 amino acids, and a total number of hydrophilic amino acids in X is more than 10. In some embodiments. X in the mutant comprises 40 to 65 amino acids, and a total number of hydrophilic amino acids in X is more than 15. In some embodiments. X in the mutant comprises 40 to 65 amino acids, and a total number of hydrophilic amino acids in X is more than 20. In some embodiments. X in the mutant comprises 40 to 65 amino acids, and a total number of hydrophilic amino acids in X is more than 25. In some embodiments. X in the mutant comprises 40 to 65 amino acids, and a total number of hydrophilic amino acids in X is more than 30. In some embodiments. X in the mutant comprises 40 to 65 amino acids, and a total number of hydrophilic amino acids in X is more than 35.

In some embodiments. X in the mutant is a moiety comprising 40 to 65 amino acids, and a total number of hydrophobic amino acids in X is no more than 15. In some embodiments. X in the mutant is a moiety comprising 40 to 65 amino acids, and a total number of hydrophobic amino acids in X is no more than 14. In some embodiments. X in the mutant is a moiety comprising 40 to 65 amino acids, and a total number of hydrophobic amino acids in X is no more than 13. In some embodiments. X in the mutant is a moiety comprising 40 to 65 amino acids, and a total number of hydrophobic amino acids in X is no more than 12. In some embodiments. X in the mutant is a moiety comprising 40 to 65 amino acids, and a total number of hydrophobic amino acids in X is no more than 11. In some embodiments. X in the mutant is a moiety comprising 40 to 65 amino acids, and a total number of hydrophobic amino acids in X is no more than 10. In some embodiments. X in the mutant is a moiety comprising 40 to 65 amino acids, and a total number of hydrophobic amino acids in X is no more than 5.

In some embodiments. X in the mutant comprises a sequence having at least 70% identity with SEQ ID NO: 1. In some embodiments. X in the mutant comprises a sequence having at least 75% identity with SEQ ID NO: 1. In some embodiments. X in the mutant comprises a sequence having at least 80% identity with SEQ ID NO: 1. In some embodiments. X in the mutant comprises a sequence having at least 85% identity with SEQ ID NO: 1. In some embodiments. X in the mutant comprises a sequence having at least 90% identity with SEQ ID NO: 1. In some embodiments. X in the mutant comprises a sequence having at least 95% identity with SEQ ID NO: 1.

In some embodiments. Y in the mutant is a moiety comprising a sequence having at least 70% identity with SEQ ID NO: 2. In some embodiments. Y in the mutant is a moiety comprising a sequence having at least 75% identity with SEQ ID NO: 2. In some embodiments. Y in the mutant is a moiety comprising a sequence having at least 80% identity with SEQ ID NO: 2. In some embodiments. Y in the mutant is a moiety comprising a sequence having at least 85% identity with SEQ ID NO: 2. In some embodiments. Y in the mutant is a moiety comprising a sequence having at least 90% identity with SEQ ID NO: 2. In some embodiments. Y in the mutant is a moiety comprising a sequence having at least 95% identity with SEQ ID NO: 2. In some embodiments. Y in the mutant is a moiety comprising a sequence having at least 96%, at least 97%, at least 98%, or at least 99% identity with SEQ ID NO: 2. In some embodiments. Y in the mutant is a moiety comprising a sequence of SEQ ID NO: 2. In some embodiments. Y in the mutant is a moiety that is a sequence of SEQ ID NO: 2.

In some embodiments. Y in the mutant is hydrophobic moiety, at least 50% amino acids of Y are selected from I, V, L, F, C, M, and A. In some embodiments. Y in the mutant is hydrophobic moiety, at least 55% amino acids of Y are selected from I, V, L, F, C, M, and A. In some embodiments. Y in the mutant is hydrophobic moiety, at least 60% amino acids of Y are selected from I, V, L, F, C, M, and A. In some embodiments. Y in the mutant is hydrophobic moiety, at least 65% amino acids of Y are selected from I. V, L, F, C, M, and A. In some embodiments. Y in the mutant is hydrophobic moiety, at least 70% amino acids of Y are selected from I, V, L, F, C, M, and A. In some embodiments. Y in the mutant is hydrophobic moiety, at least 75% amino acids of Y are selected from I, V, L, F, C, M, and A. In some embodiments. Y in the mutant is hydrophobic moiety, at least 80% amino acids of Y are selected from I, V, L, F, C, M, and A. In some embodiments. Y in the mutant is hydrophobic moiety, at least 85% amino acids of Y are selected from I. V, L, F, C, M, and A. In some embodiments. Y in the mutant is hydrophobic moiety, at least 90% amino acids of Y are selected from I, V, L, F, C, M, and A.

In another aspect, provided is a mutant of artificial polypeptide of formula (V).

In some embodiments, at least 20% amino acids of X in the mutant are selected from R, K, N, D, Q, E, and H. In some embodiments, at least 25% amino acids of X in the mutant are selected from R, K, N, D, Q, E, and H. In some embodiments, at least 30% amino acids of X in the mutant are selected from R, K, N, D, Q, E, and H. In some embodiments, at least 35% amino acids of X in the mutant are selected from R, K, N, D, Q, E, and H. In some embodiments, at least 40% amino acids of X in the mutant are selected from R, K, N, D, Q, E, and H. In some embodiments, at least 45% amino acids of X in the mutant are selected from R, K, N, D, Q, E, and H. In some embodiments, at least 50% amino acids of X in the mutant are selected from R, K, N, D, Q, E, and H. In some embodiments, at most 55% amino acids of X in the mutant are selected from R, K, N, D, Q, E, and H. In some embodiments, at most 60% amino acids of X in the mutant are selected from R, K, N, D, Q, E, and H. In some embodiments, at most 65% amino acids of X in the mutant are selected from R, K, N, D, Q, E, and H. In some embodiments, at most 70% amino acids of X in the mutant are selected from R, K, N, D, Q, E, and H. In some embodiments, at most 75% amino acids of X in the mutant are selected from R, K, N, D, Q, E, and H. In some embodiments, at most 80% amino acids of X in the mutant are selected from R, K, N, D, Q, E, and H. In some embodiments, at most 85% amino acids of X in the mutant are selected from R, K, N, D, Q, E, and H. In some embodiments, at most 90% amino acids of X in the mutant are selected from R, K, N, D, Q, E, and H.

In some embodiments. Y in the mutant comprises 10 to 25 amino acids. In some embodiments. Y in the mutant comprises 10 to 20 amino acids. In some embodiments. Y in the mutant comprises 10 to 15 amino acids. In some embodiments. Y in the mutant comprises 10, 11, 12, 13, 14, 15, 16, 17, 18, 19, 20, 21, 22, 23, 24, 25, 26, 27, 28, 29 or 30 amino acids.

In some embodiments. X in the mutant comprises a sequence having at least 70% identity with SEQ ID NO: 1. In some embodiments. X in the mutant comprises a sequence having at least 75% identity with SEQ ID NO: 1. In some embodiments. X in the mutant comprises a sequence having at least 80% identity with SEQ ID NO: 1. In some embodiments. X in the mutant comprises a sequence having at least 85% identity with SEQ ID NO: 1. In some embodiments. X in the mutant comprises a sequence having at least 90% identity with SEQ ID NO: 1. In some embodiments. X in the mutant comprises a sequence having at least 95% identity with SEQ ID NO: 1.

In some embodiments. Y in the mutant comprises a sequence having at most 50% identity with SEQ ID NO: 2. In some embodiments. Y in the mutant comprises a sequence having at most 55% identity with SEQ ID NO: 2. In some embodiments. Y in the mutant comprises a sequence having at most 60% identity with SEQ ID NO: 2. In some embodiments. Y in the mutant comprises a sequence having at most 65% identity with SEQ ID NO: 2. In some embodiments. Y in the mutant comprises a sequence having at most 70% identity with SEQ ID NO: 2. In some embodiments. Y in the mutant comprises a sequence having at most 75% identity with SEQ ID NO: 2. In some embodiments. Y in the mutant comprises a sequence having at most 80% identity with SEQ ID NO: 2. In some embodiments. Y in the mutant comprises a sequence having at most 85% identity with SEQ ID NO: 2. In some embodiments. Y in the mutant comprises a sequence having at most 90% identity with SEQ ID NO: 2.

### Formulation

In another aspect, provided herein is a formulation comprising an artificial polypeptide of the present disclosure, e.g., an artificial polypeptide of formula (VI) or (VII), or a mutant of the present disclosure, e.g., a mutant of artificial polypeptide of formula (I), (II), (III), (IV) or (V) and a pharmaceutically acceptable excipient. For the purpose of conciseness, the artificial polypeptide of the present disclosure or the mutant of the present disclosure is sometimes termed as “active ingredients” hereinafter.

The formulation as described in the present disclosure may be provided in any suitable form, depending on the route of administration. In some embodiments, the formulation is formulated for oral, buccal, topical, transdermal, rectal, intranasal, intrapulmonary, transmucosal, inhalation, or parenteral such as intravenous, intraarterial, intramuscular, subcutaneous, intraosscous or intraperitoneal administration.

In some embodiments of the formulation, the formulation is in a form suitable for oral administration. In some embodiments, the formulation is in a form of a tablet, capsule, pill, powder, granule, solution, suspension or emulsion. The formulation may comprise a predetermined amount of active ingredients.

In some embodiments, the formulation is formulated as a tablet. The tablet may comprise a coating that protects it from the acidic environment of the stomach. For example, the coating may be an enteric coating that maintains its integrity in the stomach and releases the active ingredients in the intestine. In some embodiments, the formulation is formulated as a capsule. The capsule can contain a liquid excipient such as a fatty oil. In some embodiments, the formulation is formulated as various liquid oral formulations, such as aqueous solutions, emulsions, or suspensions. In some embodiments, the liquid oral formulations are solutions and/or suspensions reconstituted from non-effervescent granules or effervescent granules. Aqucous solutions include, for example, elixirs and syrups. Elixirs are clear, sweetened, hydroalcoholic preparations. Syrups are concentrated aqueous solutions of a sugar, for example, sucrose, and may contain one or more preservatives. Emulsions are two phase systems in which one liquid is dispersed in the form of small globules throughout another liquid. Emulsions may be oil in water or water in oil emulsions. Excipients used in emulsions are non-aqueous liquids, emulsifying agents and preservatives. Suspensions are heterogeneous mixtures in which the solute particles do not dissolve, but get suspended throughout the bulk of the solvent, left floating around freely in the medium. Suspensions use suspending agents and preservatives. Acceptable substances used in non-effervescent granules, to be reconstituted into a liquid oral formulation, include diluents, sweeteners and wetting agents. Acceptable substances used in effervescent granules, to be reconstituted into a liquid oral formulation, include organic acids and a source of carbon dioxide. Coloring and flavoring agents are used in all of the above formulations.

In some embodiments of the formulation, the formulation is in a form suitable for parenteral administration. In some embodiments, the formulation is in a form of a sterile solution, suspension, emulsion, gel (e.g., injectable hydrogel) or a sterile powder for injection. The formulation may comprise a predetermined amount of active ingredients.

In some embodiments, the formulation is formulated as an injection. Examples of injections include, but not limited to a sterile solution, suspension or emulsion in aqueous or oily vehicles. Aqueous solutions in saline are conventionally used for injections. Ethanol, glycerol, propylene glycol, liquid polyethylene glycol, cyclodextrin derivatives, and vegetable oils may also be used. Oily vehicles that may be used in injections include, but are not limited to lipophilic solvents such as fatty oils or synthetic fatty acid esters. Aqueous injection suspensions may also contain substances such as humectants, suspending agents, and/or flocculating agents or deflocculating agents. The prevention of the action of microorganisms can be brought about by various antibacterial and antifungal agents, for example, parabens, chlorobutanol, phenol, sorbic acid, thimerosal, and the like. The formulation may also be formulated for bolus injection or continuous infusion. Alternatively, the formulation may be freeze-dried or in powder form for reconstitution with a suitable vehicle, e.g., sterile pyrogen-free water, immediately before use (i.e., a sterile powder for injection).

In some embodiments of the formulation, the formulation is in a form suitable for topical administration. In some embodiments, the formulation is in a form of a spray, aerosol, powder for inhalation, ointment, cream, patch, suppository, paste, pellicle or gel. The formulation may comprise a predetermined amount of active ingredients.

In some embodiments, the formulation is formulated as a topical formulation, such as for topical application to the skin and mucous membranes. A useful topical formulation for the active ingredients may be a cream, a gel, an ointment, a paste, an aerosol, a spray, a suppository or any other formulations suitable for topical administration. For example, the active ingredients may also be formulated as aerosols for topical application, such as by inhalation. These formulations for administration to the respiratory tract can be in the form of an aerosol for a nebulizer, or as a microfine powder for insufflation, alone or in combination with an inert carrier such as lactose. In such a case, the particles of the formulation will, in some embodiments, have mass median geometric diameters of less than 5 microns, in other embodiments less than 10 microns.

In some embodiments of the formulation, the formulation is in a form of sustained, controlled or delayed release formulation. In some embodiments, the formulation is in a form of a matrix tablet, compressed tablet with mixed particles having various rate of release, osmotic pump tablet, sustained or controlled release capsule or microcapsule, sustained or controlled release implant, sustained or controlled release particle, microparticle or microsphere, enteric coated capsule or tablet, colon-located formulation, gel (e.g., hydrogel), liposome, ion-exchange resin, floating formulation, bio-adhesive formulation, stimuli inducing release formulation and pulsatile drug delivery system.

In some embodiments, the formulation is formulated as a sustained release formulation. Examples of sustained release formulations include, but not limited to shaped articles (e.g., sustained release (micro) capsules) formed by semipermeable matrices of solid hydrophobic polymers. Examples of sustained release matrices include, but not limited to hydrogels, polylactides, polyesters, non-degradable ethylene-vinyl acetate, degradable lactic acid-glycolic acid copolymers, copolymers of L-glutamic acid and γ-ethyl-L-glutamatc, and poly-D-(−)-3-hydroxybutyric acid.

In some embodiments, the formulation is formulated as a controlled release formulation. Such formulations can be used to provide controlled release of one or more active ingredients using, for example, hydroxypropylmethyl cellulose, other polymer matrices, gels, permeable membranes, osmotic systems, multilayer coatings, microparticles, liposomes, microspheres, or a combination thereof to provide the desired release profile in varying proportions. All controlled release formulations have a common goal of improving drug therapy over that achieved by their non controlled counterparts. Most controlled release formulations are designed to initially release an amount of an active ingredient that promptly produces the desired preventive or therapeutic effect, and gradually and continually release of other amounts of the active ingredient to maintain this level of preventive or therapeutic effect over an extended period of time. In order to maintain this constant level of the active ingredient in the body, the active ingredient must be released from the formulation at a rate that will replace the amount of the active ingredient being metabolized and excreted from the body. Controlled release of an active ingredient can be stimulated by various conditions including, but not limited to, pH, temperature, enzymes, water, or other physiological conditions. Advantages of controlled release formulations include extended activity of the active ingredient, reduced dosage frequency, and increased patient compliance. In addition, controlled release formulations can be used to affect the time of onset of action or other characteristics, such as blood levels of the active ingredient, and can thus affect the occurrence of unwanted side effects. Suitable controlled release formulations can be readily selected for use with the active ingredients provided in the present disclosure.

In some embodiments, the formulation is a formulation for use in human.

In some embodiments, the formulation is a veterinary formulation.

In some embodiments, the formulation is formulated and administered in unit dosage forms or multiple dosage forms. Unit dose forms as used in the present disclosure refer to physically discrete units suitable for a subject in need thereof and packaged individually as is known in the art. Each unit dose contains a predetermined quantity of the active ingredients sufficient to produce the desired preventive or therapeutic effect, in association with one or more pharmaceutically acceptable excipients. Examples of unit dose forms include, but not limited to ampoules and syringes and individually packaged tablets or capsules. Unit dose forms may be administered in fractions or multiples thereof. A multiple dosage form is a plurality of identical unit dosage forms packaged in a single packaging to be administered in segregated unit dose form. Examples of multiple dosage forms include vials, bottles of tablets or capsules, or bottles of pints or gallons. Hence, a multiple dosage form is a multiple of unit doses which are not segregated in packaging.

The formulations as described in the present disclosure may be prepared using procedures well known in the art (scc, e.g., . Ansel. Introduction to Pharmaceutical Dosage Forms. Seventh Edition (1999)). The procedures typically include the step of bringing the active ingredients into association with one or more pharmaceutically acceptable excipients. In general, the formulations are prepared by uniformly and intimately admixing the active ingredients with liquid carriers or finely divided solid carriers or both, and then, if necessary, shaping the product into the desired form. For example, a tablet can be prepared by compression or molding the active ingredients, optionally with one or more pharmaceutically acceptable accessory excipients. In instances in which the active ingredients exhibit insufficient solubility, methods for solubilization may be used such as use of liposomes, prodrugs, complexation/chelation, nanoparticles, or emulsions or tertiary templating.

Any pharmaceutically acceptable excipients which are conventionally used in the pharmaceutical industry may be employed for the preparation of formulations as described in the present disclosure. The Examples of pharmaceutically acceptable excipients useful for the preparation of the formulations as described in the present disclosure may include, but not limited to: solvents, such as water, ethanol, glycerin, propylene glycol. DMSO, polyethylene glycol, fatty oils such as sesame oil, corn oil, cottonseed oil, or peanut oil, liquid paraffin, ethyl olcate, or isopropyl myristate: surfactants, such as sulfates, sulfonates, quaternary ammoniun compounds, lecithin. Spans. Tweens. Myri, polyoxyl 40 stearate, peregol O, or poloxamers: emulsifying agents, such as gelatin, acacia, tragacanth, bentonite or surfactants such as polyoxyethylene sorbitan monooleate; suspending agents, such as sodium carboxymethylcellulose, pectin, tragacanth. Vecgum or acacia: fillers, such as lactose, sucrose, trehalose, lysine, leucine, kaolin, dicalcium phosphate, mannitol, microcrystalline cellulose, or pregelatinized starch: adhesives such as celluloses. PVP, or dextrin: disintegrants, such as croscarmellose sodium, sodium starch glycolate, alginic acid, corn starch, potato starch, bentonite, methylcellulose, agar, or hydroxypropyl starch: lubricants, such as talc, polyethylene glycol, calcium stearate, magnesium stearate, lycopodium or stearic acid: glidants, such as colloidal silicon dioxide: colorants, such as any of the approved certified water soluble FD and C dyes or water insoluble FD and C dyes (e.g., those suspended on alumina hydrate): wetting agents, such as propylene glycol monostearate, sorbitan monooleate, diethylene glycol monolaurate or polyoxyethylene lauryl ether: enteric coating agents, such as fatty acids, fats, waxes, shellac, ammoniated shellac or cellulose acetate phthalates: film coating agents, such as hydroxyethylcellulose, sodium carboxymethylcellulose, polyethylene glycol 4000 or cellulose acetate phthalate: modified release agents, such as polymers such as the Eudragit R: series and cellulose esters: sweeteners, such as fructose, glucose, sucrosc. artificial sweetening agents such as sucralose, aspartame or saccharin, agave syrup, maple syrup, or corn syrup; flavoring agents, such as natural flavors extracted from plants such as fruits, peppermint, or methyl salicylate; buffers, such as citrate, phosphate, and other organic acids and/or salts thereof: antioxidants, such as ascorbic acid, methionine, citric acid. D.L-α-tocopherol. BHA. BHT, monothioglycerol, ascorbyl palmitate, ascorbic acid, or propyl gallate: preservatives, such as benzoic acid, sodium benzoate, parabens such as methyl paraben or propyl paraben, sorbic acid or benzalkonium bromide: pH modifying agents, such as citric acid, sodium citrate, hydrochloric acid. NaOH or other mild acids or bases; and chelating agents or other materials capable of binding metal ions, such as ethylene diamine tetra acetic acid (EDTA) or its salts.

The levels of active ingredients in the formulation as described in the present disclosure are effective for delivery of an amount, upon administration that treats, leads to prevention, or amelioration of one or more of the symptoms of diseases or conditions described in the present disclosure. Specifically, the amounts of the active ingredients as described in the present disclosure will be dependent on the subject being treated, the severity of the diseases or conditions, the rate of administration, the absorption, inactivation and excretion rates of the active ingredients being administered, the discretion of the prescribing physician as well as other factors known to the person skilled in the art. In some embodiments, the formulation comprises from 0.05% to 80%, from 0.1% to 50%, from 0.2% to 40%, from 0.5% to 25%, from 1% to 20%, from 5% to 15%, or from 7% to 12% (wt/wt) of the active ingredients as described in the present disclosure. The balance of the formulation is made up from one or more pharmaceutically acceptable excipients.

Alternatively, in some embodiments, the formulation comprises from about 0.001 μM to about 100 μM of the active ingredients. In some embodiments, the formulation comprises from about 0.01 μM to about 20 μM of the active ingredients. In some embodiments, the formulation comprises from about 0.1 μM to about 5 μM of the active ingredients. In some embodiments, the formulation comprises from about 0.2 μM to about 3 μM of the active ingredients. In some embodiments, the formulation comprises from about 0.1 μM to about 10 μM of the active ingredients. In some embodiments, the formulation comprises from about 1 μM to about 5 μM of the active ingredients. In some embodiments, the formulation comprises from about 1 μM to about 10 μM of the active ingredients. In some embodiments, the formulation comprises from about 5 μM to about 10 μM of the active ingredients. In some embodiments, the formulation comprises from about 10 μM to about 50 μM of the active ingredients. In some embodiments, the formulation comprises from about 20 μM to about 50 μM of the active ingredients. In some embodiments, the formulation comprises from about 5 μM to about 50 μM of the active ingredients. In some embodiments, the formulation comprises from about 1 μM to about 50 μM of the active ingredients. In some embodiments, the formulation comprises from about 1 μM to about 20 μM of the active ingredients. In some embodiments, the formulation comprises from about 5 μM to about 20 μM of the active ingredients. In some embodiments, the formulation comprises from about 10 μM to about 20 μM of the active ingredients. In some embodiments, the formulation comprises higher than about 0.001, 0.05, 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, or 3.0 μM of the active ingredients. In some embodiments, the formulation comprises lower than 100, 90, 80, 70, 60, 50, 40, 30, 20, 25, 20, 19, 18, 17, 16, 15, 14, 13, 12, 11, 10, 9, 8, 7, 6, 5, or 4 μM of the active ingredients.

### Methods of Treatment and Therapeutic Uses

In one aspect, provided is a method for treating or preventing pain in a subject in need thereof, comprising administering to the subject an effective amount of an artificial polypeptide of formula (I):

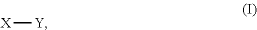

wherein X is a moiety comprising a sequence having at least 80% identity with SEQ ID NO: 1, and Y is a moiety comprising 10 to 50 amino acids, at least 50% of which are selected from I, V, L, F, C, M, and A:
wherein Y comprises a total number of Cysteine (C) of less than 5.

In some embodiments, X of formula (I) can be a hydrophilic moiety, at least 50% of which are selected from R, K, N, D, Q, E, and H. In some embodiments, X of formula (I) can be a hydrophilic moiety, at least 55% of which are selected from R, K, N, D, Q, E, and H. In some embodiments, X of formula (I) can be a hydrophilic moiety, at least 60% of which are selected from R, K, N, D, Q, E, and H. In some embodiments, X of formula (I) can be a hydrophilic moiety, at least 65% of which are selected from R, K, N, D, Q, E, and H. In some embodiments, X of formula (I) can be a hydrophilic moiety, at least 70% of which are selected from R, K, N, D, Q, E, and H. In some embodiments, X of formula (I) can be a hydrophilic moiety, at least 75% of which are selected from R. K, N, D, Q, E, and H. In some embodiments, X of formula (I) can be a hydrophilic moiety, at least 80% of which are selected from R, K, N, D, Q, E, and H. In some embodiments, X of formula (I) can be a hydrophilic moiety, at least 85% of which are selected from R. K, N, D, Q, E, and H. In some embodiments, X of formula (I) can be a hydrophilic moiety, at least 90% of which are selected from R, K, N, D, Q, E, and H.

In some embodiments, X of formula (I) can be a moiety comprising a sequence having at least 80% identity with SEQ ID NO: 1. In some embodiments, X is a moiety comprising a sequence having at least 85% identity with SEQ ID NO: 1. In some embodiments, X is a moiety comprising a sequence having at least 90% identity with SEQ ID NO: 1. In some embodiments, X is a moiety comprising a sequence having at least 95% identity with SEQ ID NO: 1. In some embodiments, X is a moiety comprising a sequence having at least 96%, at least 97%, at least 98%, or at least 99% identity with SEQ ID NO: 1. In some embodiments, X is a moiety comprising the sequence of SEQ ID NO: 1. In some embodiments, X is a moiety that is the sequence of SEQ ID NO: 1.

In some embodiments, Y of formula (I) can be a hydrophobic moiety, at least 50% of which are selected from I, V, L, F, C, M, and A. In some embodiments, Y of formula (I) can be a hydrophobic moiety, at least 55% of which are selected from I, V, L, F, C, M, and A. In some embodiments. Y of formula (I) can be a hydrophobic moiety, at least 60% of which are selected from I, V, L, F, C, M, and A. In some embodiments. Y of formula (I) can be a hydrophobic moiety, at least 65% of which are selected from I, V, L, F, C, M, and A. In some embodiments. Y of formula (I) can be a hydrophobic moiety, at least 70% of which are selected from I, V, L, F, C, M, and A. In some embodiments. Y of formula (I) can be a hydrophobic moiety, at least 75% of which are selected from I, V, L, F, C, M, and A. In some embodiments. Y of formula (I) can be a hydrophobic moiety, at least 80% of which are selected from I, V, L, F, C, M, and A. In some embodiments. Y of formula (I) can be a hydrophobic moiety, at least 85% of which are selected from I, V, L, F, C, M, and A. In some embodiments. Y of formula (I) can be a hydrophobic moiety, at least 90% of which are selected from I, V, L, F, C, M, and A.

In some embodiments. Y of formula (I) can be a moiety comprising 10 to 50 amino acids, and a total number of hydrophobic amino acids of Y is 5. In some embodiments. Y of formula (I) can be a moiety comprising 10 to 50 amino acids, and a total number of hydrophobic amino acids of Y is more than 5. In some embodiments. Y of formula (I) can be a moiety comprising 10 to 50 amino acids, and a total number of hydrophobic amino acids of Y is 6. In some embodiments. Y of formula (I) can be a moiety comprising 10 to 50 amino acids, and a total number of hydrophobic amino acids of Y is more than 6. In some embodiments. Y of formula (I) can be a moiety comprising 10 to 50 amino acids, and a total number of hydrophobic amino acids of Y is 7. In some embodiments. Y of formula (I) can be a moiety comprising 10 to 50 amino acids, and a total number of hydrophobic amino acids of Y is more than 7. In some embodiments. Y of formula (I) can be a moiety comprising 10 to 50 amino acids, and a total number of hydrophobic amino acids of Y is 8. In some embodiments. Y of formula (I) can be a moiety comprising 10 to 50 amino acids, and a total number of hydrophobic amino acids of Y is more than 8. In some embodiments. Y of formula (I) can be a moiety comprising 10 to 50 amino acids, and a total number of hydrophobic amino acids of Y is 9. In some embodiments. Y of formula (I) can be a moiety comprising 10 to 50 amino acids, and a total number of hydrophobic amino acids of Y is more than 9. In some embodiments. Y of formula (I) can be a moiety comprising 10 to 50 amino acids, and a total number of hydrophobic amino acids of Y is 10. In some embodiments. Y of formula (I) can be a moiety comprising 10 to 50 amino acids, and a total number of hydrophobic amino acids of Y is more than 10. In some embodiments. Y of formula (I) can be a moiety comprising 10 to 50 amino acids, and a total number of hydrophobic amino acids of Y is 11. In some embodiments. Y of formula (I) can be a moiety comprising 10 to 50 amino acids, and a total number of hydrophobic amino acids of Y is more than 11. In some embodiments. Y of formula (I) can be a moiety comprising 10 to 50 amino acids, and a total number of hydrophobic amino acids of Y is 12. In some embodiments. Y of formula (I) can be a moiety comprising 10 to 50 amino acids, and a total number of hydrophobic amino acids of Y is more than 12. In some embodiments. Y of formula (I) can be a moiety comprising 10 to 50 amino acids, and a total number of hydrophobic amino acids of Y is 13. In some embodiments. Y of formula (I) can be a moiety comprising 10 to 50 amino acids, and a total number of hydrophobic amino acids of Y is more than 13. In some embodiments. Y of formula (I) can be a moiety comprising 10 to 50 amino acids, and a total number of hydrophobic amino acids of Y is 14. In some embodiments. Y of formula (I) can be a moiety comprising 10 to 50 amino acids, and a total number of hydrophobic amino acids of Y is more than 14. In some embodiments. Y of formula (I) can be a moiety comprising 10 to 50 amino acids, and a total number of hydrophobic amino acids of Y is 15. In some embodiments. Y of formula (I) can be a moiety comprising 10 to 50 amino acids, and a total number of hydrophobic amino acids of Y is more than 15.

In some embodiments. Y of formula (I) can be a moiety comprising 10 to 50 amino acids, and a total number of I, V, L, F, C, M, and A of Y is 5. In some embodiments. Y of formula (I) can be a moiety comprising 10 to 50 amino acids, and a total number of I, V, L, F, C, M, and A of Y is more than 5. In some embodiments. Y of formula (I) can be a moiety comprising 10 to 50 amino acids, and a total number of I, V, L, F, C, M, and A of Y is 6. In some embodiments. Y of formula (I) can be a moiety comprising 10 to 50 amino acids, and a total number of I, V, L, F, C, M, and A of Y is more than 6. In some embodiments. Y of formula (I) can be a moiety comprising 10 to 50 amino acids, and a total number of I, V, L, F, C, M, and A of Y is 7. In some embodiments. Y of formula (I) can be a moiety comprising 10 to 50 amino acids, and a total number of I, V, L, F, C, M, and A of Y is more than 7. In some embodiments. Y of formula (I) can be a moiety comprising 10 to 50 amino acids, and a total number of I, V, L, F, C, M, and A of Y is 8. In some embodiments. Y of formula (I) can be a moiety comprising 10 to 50 amino acids, and a total number of I, V, L, F, C, M, and A of Y is more than 8. In some embodiments. Y of formula (I) can be a moiety comprising 10 to 50 amino acids, and a total number of I, V, L, F, C, M, and A of Y is 9. In some embodiments. Y of formula (I) can be a moiety comprising 10 to 50 amino acids, and a total number of I, V, L, F, C, M, and A of Y is more than 9. In some embodiments. Y of formula (I) can be a moiety comprising 10 to 50 amino acids, and a total number of I, V, L, F, C, M, and A of Y is 10. In some embodiments. Y of formula (I) can be a moiety comprising 10 to 50 amino acids, and a total number of I, V, L, F, C, M, and A of Y is more than 10. In some embodiments. Y of formula (I) can be a moiety comprising 10 to 50 amino acids, and a total number of I, V, L, F, C, M, and A of Y is 11. In some embodiments. Y of formula (I) can be a moiety comprising 10 to 50 amino acids, and a total number of I, V, L, F, C, M, and A of Y is more than 11. In some embodiments. Y of formula (I) can be a moiety comprising 10 to 50 amino acids, and a total number of I, V, L, F, C, M, and A of Y is 12. In some embodiments. Y of formula (I) can be a moiety comprising 10 to 50 amino acids, and a total number of I, V, L, F, C, M, and A of Y is more than 12. In some embodiments. Y of formula (I) can be a moiety comprising 10 to 50 amino acids, and a total number of I, V, L, F, C, M, and A of Y is 13. In some embodiments. Y of formula (I) can be a moiety comprising 10 to 50 amino acids, and a total number of I, V, L, F, C, M, and A of Y is more than 13. In some embodiments. Y of formula (I) can be a moiety comprising 10 to 50 amino acids, and a total number of I, V, L, F, C, M, and A of Y is 14. In some embodiments. Y of formula (I) can be a moiety comprising 10 to 50 amino acids, and a total number of I, V, L, F, C, M, and A of Y is more than 14. In some embodiments. Y of formula (I) can be a moiety comprising 10 to 50 amino acids, and a total number of I, V, L, F, C, M, and A of Y is 15. In some embodiments. Y of formula (I) can be a moiety comprising 10 to 50 amino acids, and a total number of I, V, L, F, C, M, and A of Y is more than 15.

In some embodiments. Y of formula (I) can be a moiety comprising 10 to 50 amino acids, and a total number of Cysteine (C) of Y is 5. In some embodiments. Y of formula (I) can be a moiety comprising 10 to 50 amino acids, and a total number of Cysteine (C) of Y is less than 5. In some embodiments. Y of formula (I) can be a moiety comprising 10 to 50 amino acids, and a total number of Cysteine (C) of Y is 4. In some embodiments. Y of formula (I) can be a moiety comprising 10 to 50 amino acids, and a total number of Cysteine (C) of Y is less than 4. In some embodiments. Y of formula (I) can be a moiety comprising 10 to 50 amino acids, and a total number of Cysteine (C) of Y is 3. In some embodiments. Y of formula (I) can be a moiety comprising 10 to 50 amino acids, and a total number of Cysteine (C) of Y is less than 3. In some embodiments. Y of formula (I) can be a moiety comprising 10 to 50 amino acids, and a total number of Cysteine (C) of Y is 2. In some embodiments. Y of formula (I) can be a moiety comprising 10 to 50 amino acids, and a total number of Cysteine (C) of Y is less than 2. In some embodiments. Y of formula (I) can be a moiety comprising 10 to 50 amino acids, and a total number of Cysteine (C) of Y is 1. In some embodiments. Y of formula (I) can be a moiety comprising 10 to 50 amino acids, and a total number of Cysteine (C) of Y is less than 1. In some embodiments. Y of formula (I) can be a moiety comprising 10 to 50 amino acids, and a total number of Cysteine (C) of Y is 0.

In some embodiments, a total number of hydrophilic amino acids in Y of formula (I) is 5. In some embodiments, a total number of hydrophilic amino acids in Y of formula (I) is no more than 5. In some embodiments, a total number of hydrophilic amino acids in Y of formula (I) is 4. In some embodiments, a total number of hydrophilic amino acids in Y of formula (I) is no more than 4. In some embodiments, a total number of hydrophilic amino acids in Y of formula (I) is 3. In some embodiments, a total number of hydrophilic amino acids in Y of formula (I) is no more than 3. In some embodiments, a total number of hydrophilic amino acids in Y of formula (I) is 2. In some embodiments, a total number of hydrophilic amino acids in Y of formula (I) is no more than 2. In some embodiments, a total number of hydrophilic amino acids in Y of formula (I) is 1. In some embodiments, a total number of hydrophilic amino acids in Y of formula (I) is no more than 1.

In some embodiments, a total number of R, K, N, D, Q, E, and H in Y of formula (I) is 5. In some embodiments, a total number of R, K, N, D, Q, E, and H in Y of formula (I) is no more than 5. In some embodiments, a total number of hydrophilic amino acids in Y of formula (I) is 4. In some embodiments, a total number of R, K, N, D, Q, E, and H in Y of formula (I) is no more than 4. In some embodiments, a total number of R, K, N, D, Q, E, and H in Y of formula (I) is 3. In some embodiments, a total number of R, K, N, D, Q, E, and H in Y of formula (I) is no more than 3. In some embodiments, a total number of R, K, N, D, Q, E, and H in Y of formula (I) is 2. In some embodiments, a total number of R, K, N, D, Q, E, and H in Y of formula (I) is no more than 2. In some embodiments, a total number of R, K, N, D, Q, E, and H in Y of formula (I) is 1. In some embodiments, a total number of R, K, N, D, Q, E, and H in Y of formula (I) is no more than 1.

In some embodiments of the artificial polypeptide. X of formula (I) comprises a sequence having at least 70% identity with any one selected from SEQ ID NOs: 81-83. In some embodiments of the artificial polypeptide. X of formula (I) comprises a sequence having at least 75% identity with any one selected from SEQ ID NOs: 81-83. In some embodiments of the artificial polypeptide. X of formula (I) comprises a sequence having at least 80% identity with any one selected from SEQ ID NOs: 81-83. In some embodiments of the artificial polypeptide. X of formula (I) comprises a sequence having at least 85% identity with any one selected from SEQ ID NOs: 81-83. In some embodiments of the artificial polypeptide. X of formula (I) comprises a sequence having at least 90% identity with any one selected from SEQ ID NOs: 81-83. In some embodiments of the artificial polypeptide. X of formula (I) comprises a sequence having at least 95% identity with any one selected from SEQ ID NOs: 81-83. In some embodiments of the artificial polypeptide. X of formula (I) comprises a sequence having at least 96%, at least 97%, at least 98%, or at least 99% identity with any onc selected from SEQ ID NOs: 81-83. In some embodiments of the artificial polypeptide. X of formula (I) comprises a sequence of any one selected from SEQ ID NOs: 81-83. In some embodiments of the artificial polypeptide. X of formula (I) is a sequence of any one selected from SEQ ID NOs: 81-83.

In some embodiments of the artificial polypeptide. Y of formula (I) comprises a sequence having at least 70% identity with any one selected from SEQ ID NOs: 3-15, 58-61 and 84-85. In some embodiments of the artificial polypeptide. Y of formula (I) comprises a sequence having at least 75% identity with any one selected from SEQ ID NOs: 3-15, 58-61 and 84-85. In some embodiments of the artificial polypeptide. Y of formula (I) comprises a sequence having at least 80% identity with any one selected from SEQ ID NOs: 3-15, 58-61 and 84-85. In some embodiments of the artificial polypeptide. Y of formula (I) comprises a sequence having at least 85% identity with any one selected from SEQ ID NOs: 3-15, 58-61 and 84-85. In some embodiments of the artificial polypeptide. Y of formula (I) comprises a sequence having at least 90% identity with any one selected from SEQ ID NOs: 3-15, 58-61 and 84-85. In some embodiments of the artificial polypeptide. Y of formula (I) comprises a sequence having at least 95% identity with any one selected from SEQ ID NOs: 3-15, 58-61 and 84-85. In some embodiments of the artificial polypeptide. Y of formula (I) comprises a sequence having at least 96%, at least 97%, at least 98%, or at least 99% identity with any one selected from SEQ ID NOs: 3-15, 58-61 and 84-85. In some embodiments of the artificial polypeptide. Y of formula (I) comprises a sequence of any one selected from SEQ ID NOs: 3-15, 58-61 and 84-85. In some embodiments of the artificial polypeptide. Y of formula (I) is a sequence of any one selected from SEQ ID NOs: 3-15, 58-61 and 84-85.

In some embodiments of the artificial polypeptide of formula (I), the polypeptide comprises a sequence having at least 70% identity with any one selected from SEQ ID NOs: 29-41, 54-57, 75 and 91-95. In some embodiments of the artificial polypeptide of formula (I), the polypeptide comprises a sequence having at least 75% identity with any one selected from SEQ ID NOs: 29-41, 54-57, 75 and 91-95. In some embodiments of the artificial polypeptide of formula (I), the polypeptide comprises a sequence having at least 80% identity with any one selected from SEQ ID NOs: 29-41, 54-57, 75 and 91-95. In some embodiments of the artificial polypeptide of formula (I), the polypeptide comprises a sequence having at least 85% identity with any one selected from SEQ ID NOs: 29-41, 54-57, 75 and 91-95. In some embodiments of the artificial polypeptide of formula (I), the polypeptide comprises a sequence having at least 90% identity with any one selected from SEQ ID NOs: 29-41, 54-57, 75 and 91-95. In some embodiments of the artificial polypeptide of formula (I), the polypeptide comprises a sequence having at least 95% identity with any one selected from SEQ ID NOs: 29-41, 54-57, 75 and 91-95. In some embodiments of the artificial polypeptide of formula (I), the polypeptide comprises a sequence having at least 96%, at least 97%, at least 98%, or at least 99% identity with any one selected from SEQ ID NOs: 29-41, 54-57, 75 and 91-95. In some embodiments of the artificial polypeptide of formula (I), the polypeptide comprises a sequence of any one of SEQ ID NOs: 29-41, 54-57, 75 and 91-95. In some embodiments of the artificial polypeptide of formula (I), the polypeptide is a sequence of any one of SEQ ID NOs: 29-41, 54-57, 75 and 91-95.

In another aspect, provided is a method for treating or preventing pain in a subject in need thereof, comprising administering to the subject an effective amount of an artificial polypeptide of formula (II):

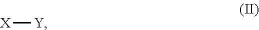

wherein X is a moiety comprising a sequence having at least 80% identity with SEQ ID NO: 1, and Y is a moiety comprising 10 to 50 amino acids, at least 50% of which are selected from I, V, L, F, C, M, and A:
wherein Y comprises a sequence having at most 90% identity with SEQ ID NO: 2.

In some embodiments. X of formula (II) can be a hydrophilic moiety, at least 50% of which are selected from R, K, N, D, Q, E, and H. In some embodiments. X of formula (II) can be a hydrophilic moiety, at least 55% of which are selected from R, K, N, D, Q, E, and H. In some embodiments. X of formula (I) can be a hydrophilic moiety, at least 60% of which are selected from R, K, N, D, Q, E, and H. In some embodiments. X of formula (II) can be a hydrophilic moiety, at least 65% of which are selected from R, K, N, D, Q, E, and H. In some embodiments. X of formula (II) can be a hydrophilic moiety, at least 70% of which are selected from R, K, N, D, Q, E, and H. In some embodiments. X of (II) can be a hydrophilic moiety, at least 75% of which are selected from R, K, N, D, Q, E, and H. In some embodiments. X of formula (II) can be a hydrophilic moiety, at least 80% of which are selected from R, K, N, D, Q, E, and H. In some embodiments. X of formula (II) can be a hydrophilic moiety, at least 85% of which are selected from R, K, N, D, Q, E, and H. In some embodiments. X of formula (II) can be a hydrophilic moiety, at least 90% of which are selected from R, K, N, D, Q, E, and H.

In some embodiments. X of formula (II) can be a hydrophilic moiety comprising one or more Arginine (R). For example. X of formula (II) can be a hydrophilic moiety comprising at least one, at least two, at least three, at least four, at least five, at least six, at least seven, at least eight, at least nine, or at least ten Arginine (R). In some embodiments. X of formula (II) can be a hydrophilic moiety comprising one or more Lysine (K). For example. X of formula (II) can be a hydrophilic moiety comprising at least one, at least two, at least three, at least four, at least five, at least six, at least seven, at least eight, at least nine, or at least ten Lysine (K). In some embodiments. X of formula (II) can be a hydrophilic moiety comprising one or more Asparaginc (N). For example. X of formula (II) can be a hydrophilic moiety comprising at least one, at least two, at least three, at least four, at least five, at least six, at least seven, at least eight, at least nine, or at least ten Asparagine (N). In some embodiments. X of formula (II) can be a hydrophilic moiety comprising one or more Aspartic Acid (D). For example. X of formula (II) can be a hydrophilic moiety comprising at least one, at least two, at least three, at least four, at least five, at least six, at least seven, at least eight, at least nine, or at least ten Aspartic Acid (D). In some embodiments. X of formula (II) can be a hydrophilic moiety comprising one or more Glutamine (Q). For example. X of formula (II) can be a hydrophilic moiety comprising at least one, at least two, at least three, at least four, at least five, at least six, at least seven, at least eight, at least nine, or at least ten Glutamine (Q). In some embodiments. X of formula (II) can be a hydrophilic moiety comprising one or more Glutamic Acid (E). For example. X of formula (II) can be a hydrophilic moiety comprising at least one, at least two, at least three, at least four, at least five, at least six, at least seven, at least eight, at least nine, or at least ten Glutamic Acid (E). In some embodiments. X of formula (II) can be a hydrophilic moiety comprising one or more Histidine (H). For example. X of formula (II) can be a hydrophilic moiety comprising at least one, at least two, at least three, at least four, at least five, at least six, at least seven, at least eight, at least nine, or at least ten Histidine (H).

In some embodiments. X of formula (II) can be a moiety comprising a sequence having at least 80% identity with SEQ ID NO: 1. In some embodiments. X is a moiety comprising a sequence having at least 85% identity with SEQ ID NO: 1. In some embodiments. X is a moiety comprising a sequence having at least 90% identity with SEQ ID NO: 1. In some embodiments. X is a moiety comprising a sequence having at least 95% identity with SEQ ID NO: 1. In some embodiments. X is a moiety comprising a sequence having at least 96%, at least 97%, at least 98%, or at least 99% identity with SEQ ID NO: 1. In some embodiments. X is a moiety comprising the sequence of SEQ ID NO: 1. In some embodiments. X is a moiety that is the sequence of SEQ ID NO: 1.

In some embodiments of the artificial polypeptide of formula (II). Y comprises a sequence having at most 50% identity with SEQ ID NO: 2. In some embodiments. Y comprises a sequence having at most 55% identity with SEQ ID NO: 2. In some embodiments. Y comprises a sequence having at most 60% identity with SEQ ID NO: 2. In some embodiments. Y comprises a sequence having at most 65% identity with SEQ ID NO: 2. In some embodiments. Y comprises a sequence having at most 70% identity with SEQ ID NO: 2. In some embodiments. Y comprises a sequence having at most 75% identity with SEQ ID NO: 2. In some embodiments. Y comprises a sequence having at most 80% identity with SEQ ID NO: 2. In some embodiments. Y comprises a sequence having at most 85% identity with SEQ ID NO: 2. In some embodiments. Y comprises a sequence having at most 90% identity with SEQ ID NO: 2.

In some embodiments of the artificial polypeptide of formula (II). Y comprises a sequence having at least 70% identity with any one selected from SEQ ID NOs: 16-18. In some embodiments of the artificial polypeptide. Y comprises a sequence having at least 75% identity with any one selected from SEQ ID NOs: 16-18. In some embodiments of the artificial polypeptide. Y comprises a sequence having at least 80% identity with any one selected from SEQ ID NOs: 16-18. In some embodiments of the artificial polypeptide. Y comprises a sequence having at least 85% identity with any one selected from SEQ ID NOs: 16-18. In some embodiments of the artificial polypeptide. Y comprises a sequence having at least 90% identity with any one selected from SEQ ID NOs: 16-18. In some embodiments of the artificial polypeptide. Y comprises a sequence having at least 95% identity with any one selected from SEQ ID NOs: 16-18. In some embodiments of the artificial polypeptide. Y comprises a sequence having at least 96%, at least 97%, at least 98%, or at least 99% identity with any one selected from SEQ ID NOs: 16-18. In some embodiments of the artificial polypeptide. Y comprises a sequence of any one selected from SEQ ID NOs: 16-18. In some embodiments of the artificial polypeptide. Y is a sequence of any one selected from SEQ ID NOs: 16-18.

In some embodiments of the artificial polypeptide of formula (II), the artificial polypeptide comprises a sequence having at least 70% identity with any one selected from SEQ ID NOs: 42-44. In some embodiments of the artificial polypeptide, the artificial polypeptide comprises a sequence having at least 75% identity with any one selected from SEQ ID NOs: 42-44. In some embodiments of the artificial polypeptide, the artificial polypeptide comprises a sequence having at least 80% identity with any one selected from SEQ ID NOs: 42-44. In some embodiments of the artificial polypeptide, the artificial polypeptide comprises a sequence having at least 85% identity with any one selected from SEQ ID NOs: 42-44. In some embodiments of the artificial polypeptide, the artificial polypeptide comprises a sequence having at least 90% identity with any one selected from SEQ ID NOs: 42-44. In some embodiments of the artificial polypeptide, the artificial polypeptide comprises a sequence having at least 95% identity with any one selected from SEQ ID NOs: 42-44. In some embodiments of the artificial polypeptide, the artificial polypeptide comprises a sequence having at least 96%, at least 97%, at least 98%, or at least 99% identity with any one selected from SEQ ID NOs: 42-44. In some embodiments of the artificial polypeptide, the artificial polypeptide comprises a sequence of any one selected from SEQ ID NOs: 42-44. In some embodiments of the artificial polypeptide, the artificial polypeptide is a sequence of any one selected from SEQ ID NOs: 42-44.

In another aspect, provided is a method for treating or preventing pain in a subject in need thereof, comprising administering to the subject an effective amount of an artificial polypeptide of formula (III):

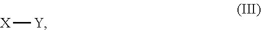

wherein X is a moiety comprising 40 to 65 amino acids, at least 50% of which are selected from H, R, K, D, Q, N and E; and
Y is a moiety comprising a sequence having at least 80% identity with SEQ ID NO: 2, wherein a total number of H, R, K, D, Q, N and E in X is less than 33.

In some embodiments. X of formula (III) is hydrophilic moiety, at least 50% of which are selected from H, R, K, D, Q, N and E. In some embodiments. X of formula (III) can be a hydrophilic moiety, at least 55% of which are selected from R, K, N, D, Q, E, and H. In some embodiments. X of formula (III) can be a hydrophilic moiety, at least 60% of which are selected from R, K, N, D, Q, E, and H. In some embodiments. X of formula (III) can be a hydrophilic moiety, at least 65% of which are selected from R, K, N, D, Q, E, and H. In some embodiments. X of formula (III) can be a hydrophilic moiety, at least 70% of which are selected from R, K, N, D, Q, E, and H. In some embodiments. X of formula (III) can be a hydrophilic moiety, at least 75% of which are selected from R, K, N, D, Q, E, and H. In some embodiments. X of formula (III) can be a hydrophilic moiety, at least 80% of which are selected from R, K, N, D, Q, E, and H. In some embodiments. X of formula (III) can be a hydrophilic moiety, at least 85% of which are selected from R, K, N, D, Q, E, and H. In some embodiments. X of formula (III) can be a hydrophilic moiety, at least 90% of which are selected from R, K, N, D, Q, E, and H.

In some embodiments. X of formula (III) is a moiety comprising 40 to 65 amino acids, and a total number of H, R, K, D, Q, N and E in X is less than 33. In some embodiments. X of formula (III) is a moiety comprising 40 to 65 amino acids, and a total number of H, R, K, D, Q, N and E in X is less than 32. In some embodiments. X of formula (III) is a moiety comprising 40 to 65 amino acids, and a total number of H, R, K, D, Q, N and E in X is less than 31. In some embodiments. X of formula (III) is a moiety comprising 40 to 65 amino acids, and a total number of H, R, K, D, Q, N and E in X is less than 30. In some embodiments. X of formula (III) is a moiety comprising 40 to 65 amino acids, and a total number of H, R, K, D, Q, N and E in X is less than 29. In some embodiments. X of formula (III) is a moiety comprising 40 to 65 amino acids, and a total number of H, R, K, D, Q, N and E in X is less than 28. In some embodiments. X of formula (III) is a moiety comprising 40 to 65 amino acids, and a total number of H, R, K, D, Q, N and E in X is less than 27. In some embodiments. X of formula (III) is a moiety comprising 40 to 65 amino acids, and a total number of H. R, K, D. Q, N and E in X is less than 26. In some embodiments. X of formula (III) is a moiety comprising 40 to 65 amino acids, and a total number of H, R, K, D, Q, N and E in X is less than 25. In some embodiments. X of formula (III) is a moiety comprising 40 to 65 amino acids, and a total number of H, R, K, D, Q, N and E in X is less than 24. In some embodiments. X of formula (III) is a moiety comprising 40 to 65 amino acids, and a total number of H, R, K, D, Q, N and E in X is less than 23. In some embodiments. X of formula (III) is a moiety comprising 40 to 65 amino acids, and a total number of H, R, K, D, Q, N and E in X is less than 22. In some embodiments. X of formula (III) is a moiety comprising 40 to 65 amino acids, and a total number of H. R, K, D. Q, N and E in X is less than 21. In some embodiments. X of formula (III) is a moiety comprising 40 to 65 amino acids, and a total number of H, R, K, D, Q, N and E in X is less than 20.

In some embodiments. X of formula (III) is a moiety comprising 40 to 65 amino acids, and a total number of hydrophilic amino acids in X is more than 10. In some embodiments. X of formula (III) is a moiety comprising 40 to 65 amino acids, and a total number of hydrophilic amino acids in X is more than 11. In some embodiments. X of formula (III) is a moiety comprising 40 to 65 amino acids, and a total number of hydrophilic amino acids in X is more than 12. In some embodiments. X of formula (III) is a moiety comprising 40 to 65 amino acids, and a total number of hydrophilic amino acids in X is more than 13. In some embodiments. X of formula (III) is a moiety comprising 40 to 65 amino acids, and a total number of hydrophilic amino acids in X is more than 14. In some embodiments. X of formula (III) is a moiety comprising 40 to 65 amino acids, and a total number of hydrophilic amino acids in X is more than 15. In some embodiments. X of formula (III) is a moiety comprising 40 to 65 amino acids, and a total number of hydrophilic amino acids in X is more than 16. In some embodiments. X of formula (III) is a moiety comprising 40 to 65 amino acids, and a total number of hydrophilic amino acids in X is more than 17. In some embodiments. X of formula (III) is a moiety comprising 40 to 65 amino acids, and a total number of hydrophilic amino acids in X is more than 18. In some embodiments. X of formula (III) is a moiety comprising 40 to 65 amino acids, and a total number of hydrophilic amino acids in X is more than 19. In some embodiments. X of formula (III) is a moiety comprising 40 to 65 amino acids, and a total number of hydrophilic amino acids in X is more than 20. In some embodiments. X of formula (III) is a moiety comprising 40 to 65 amino acids, and a total number of hydrophilic amino acids in X is more than 21. In some embodiments. X of formula (III) is a moiety comprising 40 to 65 amino acids, and a total number of hydrophilic amino acids in X is more than 22. In some embodiments. X of formula (III) is a moiety comprising 40 to 65 amino acids, and a total number of hydrophilic amino acids in X is more than 23. In some embodiments. X of formula (III) is a moiety comprising 40 to 65 amino acids, and a total number of hydrophilic amino acids in X is more than 24. In some embodiments. X of formula (III) is a moiety comprising 40 to 65 amino acids, and a total number of hydrophilic amino acids in X is more than 25.

In some embodiments. X of formula (III) is a moiety comprising 40 to 65 amino acids, and a total number of hydrophobic amino acids in X is no more than 15. In some embodiments. X of formula (III) is a moiety comprising 40 to 65 amino acids, and a total number of hydrophobic amino acids in X is no more than 14. In some embodiments. X of formula (III) is a moiety comprising 40 to 65 amino acids, and a total number of hydrophobic amino acids in X is no more than 13. In some embodiments. X of formula (III) is a moiety comprising 40 to 65 amino acids, and a total number of hydrophobic amino acids in X is no more than 12. In some embodiments. X of formula (III) is a moiety comprising 40 to 65 amino acids, and a total number of hydrophobic amino acids in X is no more than 11. In some embodiments. X of formula (III) is a moiety comprising 40 to 65 amino acids, and a total number of hydrophobic amino acids in X is no more than 10. In some embodiments. X of formula (III) is a moiety comprising 40 to 65 amino acids, and a total number of hydrophobic amino acids in X is no more than 5.

In some embodiments. X of formula (III) comprises a sequence having at least 70% identity with any one selected from SEQ ID NOs: 23-27. In some embodiments. X of formula (III) comprises a sequence having at least 75% identity with any one selected from SEQ ID NOs: 23-27. In some embodiments. X of formula (III) comprises a sequence having at least 80% identity with any one selected from SEQ ID NOs: 23-27. In some embodiments. X of formula (III) comprises a sequence having at least 85% identity with any one selected from SEQ ID NOs: 23-27. In some embodiments. X of formula (III) comprises a sequence having at least 90% identity with any one selected from SEQ ID NOs: 23-27. In some embodiments. X of formula (III) comprises a sequence having at least 95% identity with any one selected from SEQ ID NOs: 23-27. In some embodiments. X of formula (III) comprises a sequence having at least 96%, at least 97%, at least 98%, or at least 99% identity with any one selected from SEQ ID NOs: 23-27. In some embodiments. X of formula (III) comprises a sequence of any one selected from SEQ ID NOs: 23-27. In some embodiments. X of formula (III) is a sequence of any one selected from SEQ ID NOs: 23-27.

In some embodiments. Y of formula (III) is a moiety comprising a sequence having at least 70% identity with SEQ ID NO: 2. In some embodiments. Y of formula (III) is a moiety comprising a sequence having at least 75% identity with SEQ ID NO: 2. In some embodiments. Y of formula (III) is a moiety comprising a sequence having at least 80% identity with SEQ ID NO: 2. In some embodiments. Y of formula (III) is a moiety comprising a sequence having at least 85% identity with SEQ ID NO: 2. In some embodiments. Y of formula (III) is a moiety comprising a sequence having at least 90% identity with SEQ ID NO: 2. In some embodiments. Y of formula (III) is a moiety comprising a sequence having at least 95% identity with SEQ ID NO: 2. In some embodiments. Y of formula (III) is a moiety comprising a sequence having at least 96%, at least 97%, at least 98%, or at least 99% identity with SEQ ID NO: 2. In some embodiments. Y of formula (III) is a moiety comprising a sequence of SEQ ID NO: 2. In some embodiments. Y of formula (III) is a moiety that is a sequence of SEQ ID NO: 2.

In some embodiments. Y of formula (III) is hydrophobic moiety, at least 50% amino acids of Y are selected from I, V, L, F, C, M, and A. In some embodiments. Y of formula (III) is hydrophobic moiety, at least 55% amino acids of Y are selected from I, V, L, F, C, M, and A. In some embodiments. Y of formula (III) is hydrophobic moiety, at least 60% amino acids of Y are selected from I, V, L, F, C, M, and A. In some embodiments. Y of formula (III) is hydrophobic moiety, at least 65% amino acids of Y are selected from I, V, L, F, C, M, and A. In some embodiments. Y of formula (III) is hydrophobic moiety, at least 70% amino acids of Y are selected from I, V, L, F, C, M, and A. In some embodiments. Y of formula (III) is hydrophobic moiety, at least 75% amino acids of Y are selected from I, V, L, F, C, M, and A. In some embodiments. Y of formula (III) is hydrophobic moiety, at least 80% amino acids of Y are selected from I, V, L, F, C, M, and A. In some embodiments. Y of formula (III) is hydrophobic moiety, at least 85% amino acids of Y are selected from I, V, L, F, C, M, and A. In some embodiments. Y of formula (III) is hydrophobic moiety, at least 90% amino acids of Y are selected from I, V, L, F, C, M, and A.

In some embodiments, the artificial polypeptide of formula (III) comprises a sequence having at least 70% identity with any one selected from SEQ ID NOs: 49-53. In some embodiments, the artificial polypeptide of formula (III) comprises a sequence having at least 75% identity with any one selected from SEQ ID NOs: 49-53. In some embodiments, the artificial polypeptide of formula (III) comprises a sequence having at least 80% identity with any one selected from SEQ ID NOs: 49-53. In some embodiments, the artificial polypeptide of formula (III) comprises a sequence having at least 85% identity with any one selected from SEQ ID NOs: 49-53. In some embodiments, the artificial polypeptide of formula (III) comprises a sequence having at least 90% identity with any one selected from SEQ ID NOs: 49-53. In some embodiments, the artificial polypeptide of formula (III) comprises a sequence having at least 95% identity with any one selected from SEQ ID NOs: 49-53. In some embodiments, the artificial polypeptide of formula (III) comprises a sequence having at least 96%, at least 97%, at least 98%, or at least 99% identity with any one selected from SEQ ID NOs: 49-53. In some embodiments, the artificial polypeptide of formula (III) comprises a sequence of any one selected from SEQ ID NOs: 49-53. In some embodiments, the artificial polypeptide of formula (III) is a sequence of any one selected from SEQ ID NOs: 49-53.

In another aspect, provided is method for treating or preventing pain in a subject in need thereof, comprising administering to the subject an effective amount of an artificial polypeptide of formula (IV):

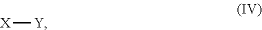

wherein X is a moiety comprising 40 to 65 amino acids, at least 50% of which are selected from H, R, K, D, Q, N and E; and
Y is a moiety comprising a sequence having at least 80% identity with SEQ ID NO: 2.
wherein X comprises a sequence having at most 90% identity with SEQ ID NO: 1.

In some embodiments. X of formula (IV) is a hydrophilic moiety, at least 50% of which are selected from H, R, K, D, Q, N and E. In some embodiments. X of formula (IV) can be a hydrophilic moiety, at least 55% of which are selected from R, K, N, D, Q, E, and H. In some embodiments. X of formula (IV) can be a hydrophilic moiety, at least 60% of which are selected from R, K, N, D, Q, E, and H. In some embodiments. X of formula (IV) can be a hydrophilic moiety, at least 65% of which are selected from R, K, N, D, Q, E, and H. In some embodiments. X of formula (IV) can be a hydrophilic moiety, at least 70% of which are selected from R, K, N, D, Q, E, and H. In some embodiments. X of formula (IV) can be a hydrophilic moiety, at least 75% of which are selected from R, K, N, D, Q, E, and H. In some embodiments. X of formula (IV) can be a hydrophilic moiety, at least 80% of which are selected from R, K, N, D, Q, E, and H. In some embodiments. X of formula (IV) can be a hydrophilic moiety, at least 85% of which are selected from R, K, N, D, Q, E, and H. In some embodiments. X of formula (IV) can be a hydrophilic moiety, at least 90% of which are selected from R, K, N, D, Q, E, and H.

In some embodiments. X of formula (IV) comprises 40 to 65 amino acids, and a total number of hydrophilic amino acids in X is more than 10. In some embodiments. X of formula (IV) comprises 40 to 65 amino acids, and a total number of hydrophilic amino acids in X is more than 15. In some embodiments. X of formula (IV) comprises 40 to 65 amino acids, and a total number of hydrophilic amino acids in X is more than 20. In some embodiments. X of formula (IV) comprises 40 to 65 amino acids, and a total number of hydrophilic amino acids in X is more than 25. In some embodiments. X of formula (IV) comprises 40 to 65 amino acids, and a total number of hydrophilic amino acids in X is more than 30. In some embodiments. X of formula (IV) comprises 40 to 65 amino acids, and a total number of hydrophilic amino acids in X is more than 35.

In some embodiments. X of formula (IV) comprises a sequence having at most 50% identity with SEQ ID NO: 1. In some embodiments. X of formula (IV) comprises a sequence having at most 55% identity with SEQ ID NO: 1. In some embodiments. X of formula (IV) comprises a sequence having at most 60% identity with SEQ ID NO: 1. In some embodiments. X of formula (IV) comprises a sequence having at most 65% identity with SEQ ID NO: 1. In some embodiments. X of formula (IV) comprises a sequence having at most 70% identity with SEQ ID NO: 1. In some embodiments. X of formula (IV) comprises a sequence having at most 75% identity with SEQ ID NO: 1. In some embodiments. X of formula (IV) comprises a sequence having at most 80% identity with SEQ ID NO: 1. In some embodiments. X of formula (IV) comprises a sequence having at most 85% identity with SEQ ID NO: 1. In some embodiments. X of formula (IV) comprises a sequence having at most 90% identity with SEQ ID NO: 1.

In some embodiments. Y of formula (IV) is a moiety comprising a sequence having at least 70% identity with SEQ ID NO: 2. In some embodiments. Y of formula (IV) is a moiety comprising a sequence having at least 75% identity with SEQ ID NO: 2. In some embodiments. Y of formula (IV) is a moiety comprising a sequence having at least 80% identity with SEQ ID NO: 2. In some embodiments. Y of formula (IV) is a moiety comprising a sequence having at least 85% identity with SEQ ID NO: 2. In some embodiments. Y of formula (IV) is a moiety comprising a sequence having at least 90% identity with SEQ ID NO: 2. In some embodiments. Y of formula (IV) is a moiety comprising a sequence having at least 95% identity with SEQ ID NO: 2. In some embodiments. Y of formula (IV) is a moiety comprising a sequence having at least 96%, at least 97%, at least 98%, or at least 99% identity with SEQ ID NO: 2. In some embodiments. Y of formula (IV) is a moiety comprising a sequence of SEQ ID NO: 2. In some embodiments. Y of formula (IV) is a moiety that is a sequence of SEQ ID NO: 2.

In some embodiments. Y of formula (IV) is hydrophobic moiety, at least 50% amino acids of Y are selected from I, V, L, F, C, M, and A. In some embodiments. Y of formula (IV) is hydrophobic moiety, at least 55% amino acids of Y are selected from I, V, L, F, C, M, and A. In some embodiments. Y of formula (IV) is hydrophobic moiety, at least 60% amino acids of Y are selected from I, V, L, F, C, M, and A. In some embodiments. Y of formula (IV) is hydrophobic moiety, at least 65% amino acids of Y are selected from I, V, L, F, C, M, and A. In some embodiments. Y of formula (IV) is hydrophobic moiety, at least 70% amino acids of Y are selected from I, V, L, F, C, M, and A. In some embodiments. Y of formula (IV) is hydrophobic moiety, at least 75% amino acids of Y are selected from I, V, L, F, C, M, and A. In some embodiments. Y of formula (IV) is hydrophobic moiety, at least 80% amino acids of Y are selected from I, V, L, F, C, M, and A. In some embodiments. Y of formula (IV) is hydrophobic moiety, at least 85% amino acids of Y are selected from I, V, L, F, C, M, and A. In some embodiments. Y of formula (IV) is hydrophobic moiety, at least 90% amino acids of Y are selected from I, V, L, F, C, M, and A.

In some embodiments. X of formula (IV) comprises a sequence having at least 70% identity with any one selected from SEQ IDs NO.19-22 and 76-79. In some embodiments. X of formula (IV) comprises a sequence having at least 75% identity with any one selected from SEQ IDs NO.19-22 and 76-79. In some embodiments. X of formula (IV) comprises a sequence having at least 80% identity with any one selected from SEQ IDs NO.19-22 and 76-79. In some embodiments. X of formula (IV) comprises a sequence having at least 85% identity with any one selected from SEQ IDs NO.19-22 and 76-79. In some embodiments. X of formula (IV) comprises a sequence having at least 90% identity with any one selected from SEQ IDs NO.19-22 and 76-79. In some embodiments. X of formula (IV) comprises a sequence having at least 95% identity with any one selected from SEQ IDs NO.19-22 and 76-79. In some embodiments. X of formula (IV) comprises a sequence having at least 96%, at least 97%, at least 98%, or at least 99% identity with any one selected from SEQ IDs NO.19-22 and 76-79. In some embodiments. X of formula (IV) comprises a sequence of any one selected from SEQ IDs NO.19-22 and 76-79. In some embodiments. X of formula (IV) is a sequence of any one selected from SEQ IDs NO. 19-22 and 76-79.

In some embodiments, the artificial polypeptide of formula (IV) comprises a sequence having at least 70% identity with any one selected from SEQ ID NOs: 45-48 and 86-89. In some embodiments, the artificial polypeptide of formula (IV) comprises a sequence having at least 75% identity with any one selected from SEQ ID NOs: 45-48 and 86-89. In some embodiments, the artificial polypeptide of formula (IV) comprises a sequence having at least 80% identity with any one selected from SEQ ID NOs: 45-48 and 86-89. In some embodiments, the artificial polypeptide of formula (IV) comprises a sequence having at least 85% identity with any one selected from SEQ ID NOs: 45-48 and 86-89. In some embodiments, the artificial polypeptide of formula (IV) comprises a sequence having at least 90% identity with any one selected from SEQ ID NOs: 45-48 and 86-89. In some embodiments, the artificial polypeptide of formula (IV) comprises a sequence having at least 95% identity with any one selected from SEQ ID NOs: 45-48 and 86-89. In some embodiments, the artificial polypeptide of formula (IV) comprises a sequence having at least 96%, at least 97%, at least 98%, or at least 99% identity with any one selected from SEQ ID NOs: 45-48 and 86-89. In some embodiments, the artificial polypeptide of formula (IV) comprises a sequence of any one selected from SEQ ID NOs: 45-48 and 86-89. In some embodiments, the artificial polypeptide of formula (IV) is a sequence of any one selected from SEQ ID NOs: 45-48 and 86-89.

In another aspect, provided is method for treating or preventing pain in a subject in need thereof, comprising administering to the subject an effective amount of an artificial polypeptide of formula (V):

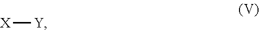

wherein X is a moiety comprising a sequence having at least 80% identity with SEQ ID NO: 1, and
Y is a moiety comprising 10 to 30 amino acids, wherein Y comprises a sequence having at least 10 continuous AAs of SEQ ID NO: 2.

In some embodiments. Y of formula (V) comprises 10 to 25 amino acids. In some embodiments. Y comprises 10 to 20 amino acids. In some embodiments. Y comprises 10 to 15 amino acids. In some embodiments. Y comprises 10, 11, 12, 13, 14, 15, 16, 17, 18, 19, 20, 21, 22, 23, 24, 25, 26, 27, 28, 29 or 30 amino acids.

In some embodiments. X of formula (V) can be a moiety comprising a sequence having at least 80% identity with SEQ ID NO: 1. In some embodiments. X is a moiety comprising a sequence having at least 85% identity with SEQ ID NO: 1. In some embodiments. X is a moiety comprising a sequence having at least 90% identity with SEQ ID NO: 1. In some embodiments. X is a moiety comprising a sequence having at least 95% identity with SEQ ID NO: 1. In some embodiments. X is a moiety comprising a sequence having at least 96%, at least 97%, at least 98%, or at least 99% identity with SEQ ID NO: 1. In some embodiments. X is a moiety comprising the sequence of SEQ ID NO: 1. In some embodiments. X is a moiety that is the sequence of SEQ ID NO: 1.

In some embodiments of the artificial polypeptide of formula (V), at least 50% amino acids of X are selected from R, K, N, D, Q, E, and H. In some embodiments, at least 55% amino acids of X are selected from R, K, N, D, Q, E, and H. In some embodiments, at least 60% amino acids of X are selected from R, K, N, D. Q, E, and H. In some embodiments, at least 65% amino acids of X are selected from R, K, N, D, Q, E, and H. In some embodiments, at least 70% amino acids of X are selected from R, K, N, D, Q, E, and H. In some embodiments, at least 75% amino acids of X are selected from R, K, N, D, Q, E, and H. In some embodiments, at least 80% amino acids of X are selected from R, K, N, D, Q, E, and H. In some embodiments, at least 85% amino acids of X are selected from R, K, N, D, Q, E, and H. In some embodiments, at least 90% amino acids of X are selected from R, K, N, D, Q, E, and H.

In some embodiments of the artificial polypeptide of formula (V). X comprises a sequence having at least 70% identity with SEQ ID NO: 96. In some embodiments of the artificial polypeptide of formula (V). X comprises a sequence having at least 75% identity with SEQ ID NO: 96. In some embodiments of the artificial polypeptide of formula (V). X comprises a sequence having at least 80% identity with SEQ ID NO: 96. In some embodiments. X comprises a sequence having at least 85% identity with SEQ ID NO: 96. In some embodiments. X comprises a sequence having at least 90% identity with SEQ ID NO: 96. In some embodiments. X comprises a sequence having at least 95% identity with SEQ ID NO: 96. In some embodiments. X comprises a sequence having at least 96%, at least 97%, at least 98%, or at least 99% identity with SEQ ID NO: 96. In some embodiments. X comprises a sequence of SEQ ID NO: 96. In some embodiments. X is a sequence of SEQ ID NO: 96.

In some embodiments of the artificial polypeptide of formula (V). Y comprises a sequence having at most 50% identity with SEQ ID NO: 2. In some embodiments. Y comprises a sequence having at most 55% identity with SEQ ID NO: 2. In some embodiments. Y comprises a sequence having at most 60% identity with SEQ ID NO: 2. In some embodiments. Y comprises a sequence having at most 65% identity with SEQ ID NO: 2. In some embodiments. Y comprises a sequence having at most 70% identity with SEQ ID NO: 2. In some embodiments. Y comprises a sequence having at most 75% identity with SEQ ID NO: 2. In some embodiments. Y comprises a sequence having at most 80% identity with SEQ ID NO: 2. In some embodiments. Y comprises a sequence having at most 85% identity with SEQ ID NO: 2. In some embodiments. Y comprises a sequence having at most 90% identity with SEQ ID NO: 2.

In some embodiments of the artificial polypeptide of formula (V). Y comprises a sequence having at least 70% identity with any one selected from SEQ ID NOs: 97-103. In some embodiments of the artificial polypeptide of formula (V). Y comprises a sequence having at least 75% identity with any one selected from SEQ ID NOs: 97-103. In some embodiments of the artificial polypeptide of formula (V). Y comprises a sequence having at least 80% identity with any one selected from SEQ ID NOs: 97-103. In some embodiments. Y comprises a sequence having at least 85% identity with any one selected from SEQ ID NOs: 97-103. In some embodiments. Y comprises a sequence having at least 90% identity with any one selected from SEQ ID NOs: 97-103. In some embodiments. Y comprises a sequence having at least 95% identity with any one selected from SEQ ID NOs: 97-103. In some embodiments. Y comprises a sequence having at least 96%, at least 97%, at least 98%, or at least 99% identity with any one selected from SEQ ID NOs: 97-103. In some embodiments. Y comprises a sequence that is any one selected from SEQ ID NOs: 97-103. In some embodiments. Y is a sequence that is any one selected from SEQ ID NOs: 97-103.

In some embodiments, the artificial polypeptide of formula (V) comprises a sequence having at least 70% identity with any one selected from SEQ ID NOs: 53-56 and 104-110. In some embodiments, the artificial polypeptide of formula (V) comprises a sequence having at least 75% identity with any one selected from SEQ ID NOs: 53-56 and 104-110. In some embodiments, the artificial polypeptide of formula (V) comprises a sequence having at least 80% identity with any one selected from SEQ ID NOs: 53-56 and 104-110. In some embodiments, the artificial polypeptide of formula (V) comprises a sequence having at least 85% identity with any one selected from SEQ ID NOs: 53-56 and 104-110. In some embodiments, the artificial polypeptide of formula (V) comprises a sequence having at least 90% identity with any one selected from SEQ ID NOs: 53-56 and 104-110. In some embodiments, the artificial polypeptide of formula (V) comprises a sequence having at least 95% identity with any one selected from SEQ ID NOs: 53-56 and 104-110. In some embodiments, the artificial polypeptide of formula (V) comprises a sequence having at least 96%, at least 97%, at least 98%, or at least 99% identity with any one selected from SEQ ID NOs: 53-56 and 104-110. In some embodiments, the artificial polypeptide of formula (V) comprises a sequence of any one selected from SEQ ID NOs: 53-56 and 104-110. In some embodiments, the artificial polypeptide of formula (V) is a sequence of any one selected from SEQ ID NOs: 53-56 and 104-110.

In another aspect, provided is a method for treating or preventing pain in a subject in need thereof, comprising administering to the subject an effective amount of an artificial polypeptide of formula (VI) or (VII), or a mutant of artificial polypeptide of formula (I), (II), (III), (IV) or (V). The exemplary embodiments of the artificial polypeptide of formula (VI) or (VII), or the mutant of artificial polypeptide of formula (I), (II), (III), (IV) or (V) are as described hereinbefore in the section entitled “ANALGESIC POLYPEPTIDE”.

In another aspect, provided is a method for treating or preventing peripheral neuropathic pain in a subject in need thereof, comprising administering to the subject an effective amount of a polypeptide having at least 70% identity with any one selected from SEQ ID NO: 28. SEQ ID NOs: 57 and 62-74 or a fragment or variant thereof.

In some embodiments, the polypeptide has at least 70% identity with any one selected from SEQ ID NO: 28. SEQ ID NOs: 57 and 62-74, or a fragment or variant thereof. In some embodiments, the polypeptide has at least 75% identity with any one selected from SEQ ID NO: 28. SEQ ID NOs: 57 and 62-74, or a fragment or variant thereof. In some embodiments, the polypeptide has at least 80% identity with any one selected from SEQ ID NO: 28. SEQ ID NOs: 57 and 62-74, or a fragment or variant thereof. In some embodiments, the polypeptide has at least 85% identity with any one selected from SEQ ID NO: 28. SEQ ID NOs: 57 and 62-74, or a fragment or variant thereof. In some embodiments, the polypeptide has at least 90% identity with any one selected from SEQ ID NO: 28. SEQ ID NOs: 57 and 62-74, or a fragment or variant thereof. In some embodiments, the polypeptide has at least 95% identity with any one selected from SEQ ID NO: 28. SEQ ID NOs: 57 and 62-74, or a fragment or variant thereof. In some embodiments, the polypeptide has at least 96%, at least 97%, at least 98%, or at least 99% identity with any one selected from SEQ ID NO: 28. SEQ ID NOs: 57 and 62-74, or a fragment or variant thereof. In some embodiments, the polypeptide comprises or is an amino acid sequence of any one selected from SEQ ID NO: 28. SEQ ID NOs: 57 and 62-74, or a fragment or variant thereof. In some embodiments, the polypeptide is a polypeptide of any one of SEQ ID NO: 28. SEQ ID NOs: 57 and 62-74. In some embodiments, the polypeptide is a polypeptide of any one of SEQ ID NOs: 28 and 62-74. In some embodiments, the polypeptide is a polypeptide of SEQ ID NO: 28.

In another aspect, provided is an artificial polypeptide of formula (VI) or (VII), or a mutant of artificial polypeptide of (I), (II), (III), (IV) or (V), for use in therapy.

In another aspect, provided is an artificial polypeptide of formula (I), (II), (III), (IV), (V). (VI), or (VII), or a mutant of artificial polypeptide of (I), (II), (III), (IV) or (V), for use in treating or preventing pain in a subject in need thereof.

In another aspect, provided is a polypeptide having at least 70% identity with any one selected from SEQ ID NO: 28. SEQ ID NOs: 57 and 62-74 or a fragment or variant thereof, for use in treating or preventing peripheral neuropathic pain in a subject in need thereof.

In another aspect, provided is use of an artificial polypeptide of formula (I), (II), (III), (IV), (V). (VI), or (VII), or a mutant of artificial polypeptide of (I), (II), (III), (IV) or (V) for the preparation of a medicament for treating or preventing pain in a subject in need thereof.

In another aspect, provided is use of a polypeptide having at least 70% identity with any one selected from SEQ ID NO: 28. SEQ ID NOs: 57 and 62-74 or a fragment or variant thereof for the preparation of a medicament for treating or preventing peripheral neuropathic pain in a subject in need thereof.

In some embodiments, the medicament is a medicament for use in human.

In some embodiments, the medicament is a veterinary medicament.

In some embodiments, the polypeptide or the mutant of the present disclosure, e.g., an artificial polypeptide of formula (I), (II), (III), (IV), (V), (VI) or (VII), or a mutant of an artificial polypeptide of formula (I), (II), (III), (IV) or (V), or a polypeptide having at least 70% identity (e.g., at least 80% identity) with any one selected from SEQ ID NO: 28. SEQ ID NOs: 57 and 62-74 or a fragment or variant thereof can exhibit superior analgesic effects and/or significantly improve the pain threshold of animals, as evidenced from the multiple pain related models (i.e., HFD (high-fat diet) plus STZ (streptozocin) model. VZV (varicella-zoster virus) model. SNL (spinal nerve ligation) model, bone pain model induced by Walker 256 breast cancer cells and cancer pain associated with chemotherapy-induced peripheral neuropathy model) provided in the Examples.

In some embodiments, the treatment with the polypeptide or the mutant of the present disclosure, e.g., an artificial polypeptide of formula (I), (II), (III), (IV), (V). (VI) or (VII), or a mutant of an artificial polypeptide of formula (I), (II), (III), (IV) or (V), or a polypeptide having at least 70% identity (e.g., at least 80% identity) with any one selected from SEQ ID NO: 28. SEQ ID NOs: 57 and 62-74 or a fragment or variant thereof results in alleviation or amelioration of pain in a subject. In some embodiment, the pain of the subject is reduced in severity or duration by about 2%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100% following administration of an effective amount of the polypeptide or the mutant of the present disclosure to the subject. In some embodiments, the pain of the subject is reduced in severity or duration by about 2-fold, about 5-fold, about 10-fold, about 15-fold, about 20-fold, about 25-fold, about 30-fold, about 35-fold, about 40-fold, about 45-fold, about 50-fold, about 55-fold, about 60-fold, about 65-fold, about 70-fold, about 75-fold, about 80-fold, about 85-fold, about 90-fold, about 95-fold, about 100-fold, or more, following administration of an effective amount of the polypeptide or the mutant of the present disclosure to the subject.

In some embodiments, the treatment with the polypeptide or the mutant of the present disclosure, e.g., an artificial polypeptide of formula (I), (II), (III), (IV), (V). (VI) or (VII), or a mutant of an artificial polypeptide of formula (I), (II), (III), (IV) or (V), or a polypeptide having at least 70% identity (e.g., at least 80% identity) with any one selected from SEQ ID NO: 28. SEQ ID NOs: 57 and 62-74 or a fragment or variant thereof results in increase or enhancement of pain threshold in a subject. In some embodiment, the pain threshold of the subject is increased or enhanced by about 2%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100% following administration of an effective amount of the polypeptide or the mutant of the present disclosure to the subject. In some embodiments, the pain threshold of the subject is increased or enhanced by about 2-fold, about 5-fold, about 10-fold, about 15-fold, about 20-fold, about 25-fold, about 30-fold, about 35-fold, about 40-fold, about 45-fold, about 50-fold, about 55-fold, about 60-fold, about 65-fold, about 70-fold, about 75-fold, about 80-fold, about 85-fold, about 90-fold, about 95-fold, about 100-fold, or more, following administration of an effective amount of the polypeptide or the mutant of the present disclosure to the subject.

In some embodiments, the treatment comprising administrating an effective amount of the polypeptide or the mutant of the present disclosure to the subject. In some embodiments, an effective amount of the polypeptide or the mutant of the present disclosure is from about 0.01 μg to about 1000 mg. In some embodiments, an effective amount of the polypeptide or the mutant of the present disclosure is at least 0.01 μg, 0.02μ, 0.05μ, 0.1μ, 0.2μ, 0.5μ, 1μ, 2μ, 5μ, 10μ, 50μ, 100μ, 200μ, 500μ, 1 mg, 2 mg, 5 mg, 10 mg, 20 mg, 50 mg, 100 mg, 200 mg, 300 mg, 400 mg, 500 mg, 600 mg, 700 mg, 800 mg, 900 mg or 1000 mg. In some embodiments, an effective amount of the polypeptide or the mutant of the present disclosure is at most 0.01 μg, 0.02 μg, 0.05 μg, 0.1 μg, 0.2 μg, 0.5 μg, 1 μg, 2 μg, 5 μg, 10 μg, 50 μg, 100 μg, 200 μg, 500 μg, 1 mg, 2 mg, 5 mg, 10 mg, 20 mg, 50 mg, 100 mg, 200 mg, 300 mg, 400 mg, 500 mg, 600 mg, 700 mg, 800 mg, 900 mg or 1000 mg. In some embodiments, an effective amount of the polypeptide or the mutant of the present disclosure is from about 0.01 μg/kg to about 100 mg/kg. In some embodiments, an effective amount of the polypeptide or the mutant of the present disclosure is at least 0.01 μg/kg, 0.02 μg/kg, 0.05 μg/kg, 0.1 μg/kg, 0.2 μg/kg, 0.5 μg/kg, 1 μg/kg, 2 μg/kg, 5 μg/kg, 10 μg/kg, 50 μg/kg, 100 μg/kg, 200 μg/kg, 500 μg/kg. 1 mg/kg, 2 mg/kg, 5 mg/kg, 10 mg/kg, 20 mg/kg, 50 mg/kg or 100 mg/kg. In some embodiments, an effective amount of the polypeptide or the mutant of the present disclosure is at most 0.01 μg/kg, 0.02 μg/kg, 0.05 μg/kg. 0.1 μg/kg, 0.2 μg/kg, 0.5 μg/kg, 1 μg/kg, 2 μg/kg, 5 μg/kg, 10 μg/kg, 50 μg/kg, 100 μg/kg, 200 μg/kg, 500 μg/kg, 1 mg/kg, 2 mg/kg, 5 mg/kg, 10 mg/kg, 20 mg/kg, 50 mg/kg or 100 mg/kg.

In some embodiments, the method comprises administrating an effective amount of the polypeptide or the mutant of the present disclosure to the subject once daily. In some embodiments, the method comprises administrating an effective amount of polypeptide or the mutant of the present disclosure to the subject twice daily. In some embodiments, the method comprises administrating an effective amount of the polypeptide or the mutant of the present disclosure to the subject three or more times daily. In some embodiments, the method comprises administrating an effective amount of the polypeptide or the mutant of the present disclosure to the subject every two days. In some embodiments, the method comprises administrating an effective amount of the polypeptide or the mutant of the present disclosure to the subject every three days. In some embodiments. the method comprises administrating an effective amount of the polypeptide or the mutant of the present disclosure to the subject weekly. In some embodiments, the method comprises administrating an effective amount of the polypeptide or the mutant of the present disclosure to the subject biweekly. In some embodiments, the method comprises administrating an effective amount of the polypeptide or the mutant of the present disclosure to the subject monthly.

In some embodiments, the pain is at least one selected from the group consisting of acute pain, chronic pain and transitional pain.

In some embodiments, the acute pain is at least one selected from the group consisting of pain associated with acute injuries, acute inflammatory pain and headache.

In some embodiments, the pain associated with acute injuries is acute postoperative pain.

In some embodiments, the chronic pain is at least one selected from the group consisting of nociceptive pain, neuropathic pain and mixed pain.

In some embodiments, the nociceptive pain is at least one selected from the group consisting of abdominal pain, anal fissure pain, bladder pain, complex regional pain syndrome, mastalgia, inhntenterospasm, bladder pain syndrome, arthralgia, musculoskeletal pain, muscle pain, myofascial pain syndrome, nociceptive bone pain, pain associated with pancreatitis, polymyalgia rheumatica, chronic postoperative pain, renal pain, somatic pain, teinodynia, desmalgia, chronic traumatic pain, pain associated with fracture and visceral pain. In some embodiments, the mastalgia is cyclic mastalgia.

In some embodiments, the renal pain is renal colic.

In some embodiments, the pain associated with fracture is pain associated with spine fracture.

In some embodiments, the mixed pain is at least one selected from the group consisting of arthritis pain, back pain, cancer pain, toothache, fibromyalgia, chronic inflammatory pain, lumbago, cervicodynia and eye pain.

In some embodiments, the arthritis pain is at least one selected from the group consisting of osteoarthritis pain, rheumatoid arthritis pain and gout pain.

In some embodiments, the cancer pain is cancer pain associated with tumor.

In some embodiments, the cancer pain associated with tumor is at least one selected from the group consisting of nerve pain associated with cancer, bone pain associated with cancer, soft tissue pain associated with cancer and referred pain associated with cancer.

In some embodiments, the bone pain associated with cancer is at least one selected from the group consisting of bone pain associated with bone cancers and bone pain associated with bone metastatic tumors.

In some embodiments, the bone pain associated with bone cancers is at least one selected from the group consisting of bone pain associated with chondrosarcoma, bone pain associated with Ewing's sarcoma, bone pain associated with malignant fibrous histiocytoma of bone, bone pain associated with osteosarcoma and bone pain associated with fibrocartilaginous mesenchymoma of bone.

In some embodiments, the cancer pain is cancer pain associated with diagnostic and/or therapeutic procedures.

In some embodiments, the cancer pain associated with diagnostic and/or therapeutic procedures is phantom pain associated with cancer. In some embodiments, the cancer pain associated with diagnostic and/or therapeutic procedures is cancer pain associated with chemotherapy. In some embodiments, the cancer pain associated with chemotherapy is cancer pain associated with chemotherapy-induced peripheral neuropathy (CIPN). In some embodiments, the chemotherapy is performed by administration of a cytotoxic agent to a subject in need thereof. In some embodiments, the cytotoxic agent is at least one selected from the group consisting of platinum anticancer agents (e.g., cisplatin, carboplatin, nedaplatin and oxaliplatin), vinca alkaloids (e.g., vinblastine, vinorelbine, vincristine and vindesine), taxanes (e.g., paclitaxel and docetaxel) and proteasome or angiogenesis inhibitors (e.g., bortezomib, carfilzomib and thalidomide).

In some embodiments, the eye pain is at least one selected from the group consisting of ocular nociceptive pain and ocular neuropathic pain.

In some embodiments, the ocular nociceptive pain is at least one selected from the group consisting of eye pain associated with injury, eye pain associated with surgery, eye pain associated with contact lenses wearing and eye pain associated with irritation of foreign bodies.

In some embodiments, the ocular neuropathic pain is at least one selected from the group consisting of eye pain associated with allergies, eye pain associated with infection, eye pain associated with inflammation, eye pain associated with chronic ocular surface disease, post-surgical ocular neuropathic pain, eye pain associated with toxic keratopathy, eye pain associated with radiation, eye pain associated with ultraviolet light exposure, eye pain associated with a systemic neuropathy, traumatic ocular neuropathic pain, eye pain with trigeminal neuralgia and eye pain associated with fibromyalgia. In some embodiments, the infection is at least one selected from the group consisting of infection caused by herpes simplex keratitis and infection caused by herpes zoster keratitis. In some embodiments, the chronic ocular surface disease is at least one selected from the group consisting of dry eye syndrome, recurrent corneal erosions, chemical burns and ocular surface neoplasia. In some embodiments, the surgery which causes post-surgical ocular neuropathic pain is at least one selected from the group consisting of kerato-refractive surgery, cataract surgery and corneal transplant surgery. In some embodiments, the toxic keratopathy is a topical toxic keratopathy, such as topical toxic keratopathy associated with preservatives containing benzalokium chloride, or a systemic toxic keratopathy, such as systemic toxic keratopathy associated with isotretinoin. In some embodiments, the systemic neuropathy is at least one selected from the group consisting of diabetes, small fiber neuropathy and multiple sclerosis.

In some embodiments, the ocular neuropathic pain is at least one selected from the group consisting of corneal neuralgia, conjunctival neuralgia, optic nerve neuralgia, extraocular muscle neuralgia, orbital neuralgia and eyelid neuralgia.

In some embodiments, the neuropathic pain is at least one selected from the group consisting of central neuropathic pain and peripheral neuropathic pain.

In some embodiments, the central neuropathic pain is at least one selected from the group consisting of post-stroke neuropathic pain, syringomyelia pain, neuropathic pain associated with ischemic myelopathy, neuropathic pain associated with oppressed myelopathy, neuropathic pain associated with radiation myelopathy, neuropathic pain associated with spinal cord injury, neuropathic pain associated with multiple sclerosis, neuropathic pain associated with Parkinson's disease, phantom limb pain and neuropathic pain associated with myelitis.

In some embodiments, the peripheral neuropathic pain is at least one selected from the group consisting of peripheral neuropathic pain associated with metabolic disorder or ischemia, peripheral neuropathic pain associated with infection, peripheral neuropathic pain associated with nerve or nerve root compression, peripheral neuropathic pain associated with chemotherapy, peripheral neuropathic pain associated with radiotherapy, stump pain, peripheral neuropathic pain associated with neuropathy due to tumor compression or infiltration, peripheral neuropathic pain associated with alcoholic polyneuropathy, peripheral neuropathic pain associated with dystrophic neuropathy, peripheral neuropathic pain associated with toxic peripheral neuropathy and peripheral neuropathic pain associated with immune neuropathy.

In some embodiments, the peripheral neuropathic pain associated with metabolic disorder or ischemia is at least one selected from the group consisting of diabetic peripheral neuropathic pain and ischemic peripheral neuropathic pain.

In some embodiments, the peripheral neuropathic pain associated with infection is at least one selected from the group consisting of peripheral neuropathic pain associated with viral infection and peripheral neuropathic pain associated with spirochetal infection.

In some embodiments, the peripheral neuropathic pain associated with viral infection is at least one selected from the group consisting of post-herpetic neuralgia and peripheral neuropathic pain associated with HIV infection.

In some embodiments, the peripheral neuropathic pain associated with spirochetal infection is peripheral neuropathic pain associated with syphilis infection.

In some embodiments, the peripheral neuropathic pain associated with nerve or nerve root compression is at least one selected from the group consisting of sciatica, trigeminal neuralgia, glossopharyngeal neuralgia, neuropathic pain associated with radiculoneuropathy and neuropathic pain associated with entrapment neuropathy.

In some embodiments, the subject is a mammal. In some embodiments, the subject is any one selected from the group consisting of a human, primate, rodent, canine, feline, equine, ovine and porcine. In some embodiments, the subject is a human. In some embodiments, the subject is an infant, a toddler, a child, an adolescent, an adult, or an elderly. In some embodiments, the subject is a man or a woman. In some embodiments, the subject is a pet. In some embodiments, the subject is any one selected from the group consisting of fish, frog, salamander, reptile (e.g., turtle, lizard, snake and iguana), bird (e.g., a parrot), mice, rat, guinea pig, gerbil, hamster, chinchilla, rabbit, ferret, cat, dog and pig.

In some embodiments, the polypeptide or the mutant of the present disclosure, e.g., an artificial polypeptide of formula (I), (II), (III), (IV), (V), (VI) or (VII), or a mutant of an artificial polypeptide of formula (I), (II), (III), (IV) or (V), or a polypeptide having at least 70% identity (e.g., at least 80% identity) with any one selected from SEQ ID NO: 28, SEQ ID NOs: 57 and 62-74 or a fragment or variant thereof is administered in the form of a formulation comprising the polypeptide or the mutant of the present disclosure and a pharmaceutically acceptable excipient.

In some embodiments, the formulation is in a form suitable for oral administration. In some embodiments, the formulation is in a form of a tablet, capsule, pill, powder, granule, solution, suspension or emulsion.

In some embodiments, the formulation is in a form suitable for parenteral administration. In some embodiments, the formulation is in a form of a sterile solution, suspension, emulsion, gel (e.g., injectable hydrogel) or a sterile powder for injection.

In some embodiments, the formulation is in a form suitable for topical administration. In some embodiments, the formulation is in a form of a spray, aerosol, powder for inhalation, ointment, cream, patch, suppository, paste, pellicle or gel.

In some embodiments, the formulation is in a form of sustained, controlled or delayed release formulation. In some embodiments, the formulation is in a form of a matrix tablet, compressed tablet with mixed particles having various rate of release, osmotic pump tablet, sustained or controlled release capsule or microcapsule, sustained or controlled release implant, sustained or controlled release particle, microparticle or microsphere, enteric coated capsule or tablet, colon-located formulation, gel (e.g., hydrogel), liposome, ion-exchange resin, floating formulation, bio-adhesive formulation, stimuli inducing release formulation and pulsatile drug delivery system.

In some embodiments, the formulation is a formulation for use in human.

In some embodiments, the formulation is a veterinary formulation.

More details of these formulations are as described hereinbefore in the section entitled “FORMULATION”.

### Preparation of the Polypeptide

The polypeptide or the mutant of the present disclosure, e.g., an artificial polypeptide of formula (I), (II), (III), (IV). (V). (VI) or (VII), or a mutant of an artificial polypeptide of formula (I), (II), (III), (IV) or (V), or a polypeptide having at least 70% identity (e.g., at least 80% identity) with any one selected from SEQ ID NO: 28, SEQ ID NOs: 57 and 62-74 or a fragment or variant thereof can be prepared by any suitable method. including but not limited to molecular cloning techniques and synthetic procedures. Standard molecular cloning techniques are well known in the art and are described by Sambrook. J., Fritsch, E. F, and Maniatis. T. Molecular Cloning: A Laboratory Manual: Cold Spring Harbor Laboratory Press: Cold Spring Harbor. (1989) (Maniatis) and by T. J. Silhavy. M. L. Bennan, and L. W. Enquist. Experiments with Gene Fusions. Cold Spring Harbor Laboratory. Cold Spring Harbor. N.Y. (1984) and by Ausubel. F. M, et al., Current Protocols in Molecular Biology, pub, by Greene Publishing Assoc, and Wiley-Interscience (1987).

In some embodiments, the polypeptide or the mutant of the present disclosure is prepared by reference to the fermentation-based manufacturing method as disclosed in Chinese patent application No. 201711320516.4 (Publication No.: CN109913483A), which is herein incorporated by reference in its entirety. In some embodiments, the method for preparing the polypeptide or the mutant of the present disclosure comprises the steps of: integrating a target gene fragment into an expression plasmid by means of genetic engineering, with the integrated target gene fragment comprising at least one purification tag: transforming the expression plasmid into a corresponding expression host to construct a recombinant engineered cell which highly expresses the target polypeptide: subjecting the recombinant engineered cell to fermentation, induced expression, and then crude purification to obtain a crude polypeptide: subjecting the crude polypeptide to refined purification to obtain highly purified polypeptide.

In some embodiments, the target gene fragment is any one selected from the group consisting of gene fragments which are capable of encoding the polypeptide or the mutant of the present disclosure, e.g., an artificial polypeptide of formula (I), (II), (III), (IV), (V). (VI) or (VII), or a mutant of an artificial polypeptide of formula (I), (II), (III), (IV) or (V), or a polypeptide having at least 70% identity (e.g., at least 80% identity) with any one selected from SEQ ID NO: 28. SEQ ID NOs: 57 and 62-74 or a fragment or variant thereof. In some embodiments, the target gene fragment is a gene fragment which is capable of encoding an artificial polypeptide of formula (I), (II), (III), (IV), (V). (VI) or (VII). In some embodiments, the target gene fragment is a gene fragment which is capable of encoding a mutant of an artificial polypeptide of formula (I), (II), (III), (IV) or (V). In some embodiments, the target gene fragment is a gene fragment which is capable of encoding a polypeptide having at least 70% identity (e.g., at least 80% identity) with any one selected from SEQ ID NO: 28. SEQ ID NOs: 57 and 62-74 or a fragment or variant thereof. The target gene fragment can be prepared by any suitable method, including but not limited to enzymatic synthesis, i.e., using RNA as a template to synthesize cDNA by reverse transcription, and chemical synthesis method, i.e., using a chemical method or chemical method combined with enzymatic method to synthesize target gene. The preparation of target gene fragment may also be commercially conducted by a contract research organization (CRO) in case that the sequence of target gene fragment is provided.

In some embodiments, the purification tag is a ST sequence tag (an amino acid sequence that helps the polypeptide or the mutant of the present disclosure to form inclusion bodies) or a His tag.

In some embodiments, the expression host is a host cell. The host cell includes but is not limited to an individual cell, cell culture, or cell line. In some embodiments, the host cells include progeny of a single host cell. In some embodiments, a host cell can be transfected with a heterologous sequence including vectors encoding the polypeptide or the mutant of the present disclosure. In some embodiments, said host cells may be prokaryotic cells, such as bacterial cells. In some embodiments, said host cells may be eukaryotic cells, such as yeast cells, animal cells, insect cells, plant cells and the like.

Examples of bacterial host cells that can be used to produce the polypeptide or the mutant of the present disclosure include microorganisms belonging to the genus Escherichia. Serratia. Bacillus. Brevibacterium. Corynebacterium. Microbacterium. Pseudomonas and the like. For example, bacterial host cells may include, but not be limited to, Escherichia coli XLI-Blue. XL2-Blue. DHI. MC 1000. KY3276. W1485. JM109. HB101. No. 49, i W3110. NY49. G1698. BL21, or TBI. Other bacterial host cells may include, but not be limited to, Serratia ficaria. Serratia fonticola. Serratia liquefaciens. Serratia marcescens. Bacillus subtilis. Bacillus amyloliquefaciens. Brevibacterium ammoniagenes. Brevibacterium immariophilum ATCC 14068.

Brevibacterium saccharolyticum ATCC 14066. Brevibacterium flavum ATCC 14067. Brevibacterium lactofermentum ATCC 13869. Corynebacterium glutamicum ATCC 13032. Corynebacterium glutamicum ATCC 13869. Corynebacterium acetoacidophilum ATCC 13870. Microbacterium ammoniaphilum ATCC 15354. Pseudomonas putida. Pseudomonas sp. D-0110 and the like.

Examples of yeast cells that can be used to produce the polypeptide or the mutant of the present disclosure include microorganisms belonging to the genus Kluyveromyces. Trichosporon. Saccharomyces. Schizosaccharomyces. Schwanniomyces. Pichia. Candida and the like, such as Saccharomyces cerevisiae. Schizosaccharomyces pombe. Kluyveromyces lactis. Trichosporon pullulans. Schwanniomyces alluvius. Candida utilis and the like.

Examples of animal cells that can be used to produce the polypeptide or the mutant of the present disclosure include mammalian cells, for example, Chinese hamster ovary cells (CHO) or monkey cells, such as COS cells, HepG2 cells, A549 cells, and any other cells available through ATCC or other depositories.

In some embodiments, the expression host is an Escherichia Coli host cell. In some embodiments, the formula of the fermentation medium used in the fermentation process is: yeast extract powder 10-50 g/L, peptone 10-30 g/L, ammonium sulfate 2-10 g/L, sodium chloride 2-10 g/L, potassium dihydrogen phosphate 0-10 g/L, dipotassium hydrogen phosphate 2-15 g/L, defoamer 0.01-0.1% (v/v), FeSO+7H₂O 0-0.1 g/L, ZnSO+7H₂O 0-0.02 g/L, CuSO+5H₂O 0-0.1 g/L, MnSO+5H₂O 0-0.05 g/L, CaCl,7H₂O 0-0.01 g/L, CoCl₂.·6H₂O 0-0.01 g/L. NaMoO+2HO 0-0.01 g/L, H₃BO₃ 0-0.0005 g/L, and Biotin 0-0.005 g/L. In some embodiments, the fermentation process is performed at 37° C.

In some embodiments, the induced expression is realized by addition of isopropyl-beta-D-thiogalactopyranoside (IPTG) during the fermentation process. In some embodiments, the IPTG is added 0.5, 1, 2, 3, 4 or 5 hours after the initiation of the fermentation process. In some embodiments, the IPTG is added at a final concentration of 0.25, 0.5, 1, 2 or 4 mM. In some embodiments, the induced expression is performed at 37° C.

The crude purification of the present disclosure is a process during which the culture produced by the fermentation process is preliminary treated. In some embodiments, the crude purification comprises the steps of: collecting the cells and isolating the inclusion body proteins and/or cytoplasmic proteins after lysing the cells: subjecting the inclusion body proteins and/or cytoplasmic proteins to denaturation, renaturation and enzymatic digestion to obtain a crude product containing the crude polypeptide. In some embodiments, the crude purification comprises the steps of: collecting the culture medium, removing the cells and impurities, and obtaining the supernatant, i.e., a crude product containing the crude polypeptide.

The refined purification of the present disclosure is a process during which the crude product containing the crude polypeptide is purified with a chromatographic method. Examples of chromatographic methods that can be used to purify the polypeptide or the mutant of the present disclosure include: ion exchange chromatography with a strong anion exchange resin, a weak anion exchange resin or a multimodal anion exchange resin: affinity chromatography: reversed phase chromatography with reversed phase packing materials: molecular sieve chromatography with size exclusion packing materials; and hydrophobic chromatography with hydrophobic packing materials.

More detailed information for preparing the polypeptide of the present disclosure may be found throughout of the disclosure of CN109913483A, e.g., Examples 1-4.

### Kit

The present disclosure also provides a kit for treating or preventing pain, e.g., peripheral neuropathic pain in a subject in need thereof. In some embodiments, the kit comprises one or more of the polypeptide or the mutant of the present disclosure, and an instruction for using the kit. In some embodiments, the kit comprises one or more formulations described herein, and an instruction for using the kit.

The kits can comprise one or more containers that contain one or more of the polypeptide or the mutant of the present disclosure or a formulation comprising the same. The polypeptide or the mutant of the present disclosure can be present in the container as a prepared formulation, or alternatively, the polypeptide or the mutant of the present disclosure can be unformulated. In some embodiments, the kit can include the unformulated polypeptide or the mutant of the present disclosure in a container that is separate from the pharmaceutically acceptable excipients. Prior to use, the polypeptide of the present disclosure is diluted or otherwise mixed with the pharmaceutically acceptable excipients.

In some embodiments, the kit provided herein also comprises instructions which describe the method for administering the formulations. In some embodiments, the instructions also describe the procedure for mixing the polypeptide or the mutant of the present disclosure contained in the kit with pharmaceutically acceptable excipients.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

### EXAMPLES

The present disclosure will now be described with reference to the following Examples, which are intended to illustrate, but not limit, the invention.

Unless otherwise specified, if the specific conditions are not indicated in the Examples, it is carried out in accordance with the conventional conditions or the conditions suggested by the manufacturer. The reagents or instruments used without the manufacturer's indication are conventional products that commercially available. Those skilled in the art would understand that the Examples described herein are not intended to limit the scope of the invention as claimed.

### Example 1: Effect of the Polypeptide of the Present Disclosure Administered Via Subcutaneous Route on the Treatment of VZV-Induced Pain Model in Rats

In this Example, the effect of an exemplary polypeptide of the present disclosure, i.e. SEQ ID NO: 29 administered via subcutaneous (s.c.) route on the treatment of pain was investigated in VZV (varicella-zoster virus)-induced pain model in rats.

### Modeling

A summary of the method for inducing pain model in rats by means of subcutaneous VZV inoculation is provided hereinafter. Briefly, male Sprague Dawley (SD) rats were deeply anesthetized by intraperitoneal injection of a Zoletil R: 50 (30 mg/kg)/xylazine (6 mg/kg) anesthetic at an injection volume of 6 mL/kg, and then the virus inoculation was performed by subcutaneous injection at the footpad area. The infection dose was 22800 TCID50 per animal, and the infection volume was 150 μL for all rats.

### Grouping and Administration

The rats were randomly divided into 2 groups, with 8 rats in the placebo group (Group 1, administered with vehicle (normal saline)) and 8 rats in the treatment group (Group 2, administered with SEQ ID NO: 29).

Administration was performed on alternate days. Briefly, the test articles were administered by subcutaneous injection at the footpad area on Day 1 and Day 3, respectively. As for Group 2, the administration of SEQ ID NO: 29 was initiated from the first concentration. Namely, the first concentration (10 μg/mL) was administered on Day 1, and the second concentration (20 μg/mL) was administered on Day 3. Pain was measured once after each administration.

More detailed information of the study is shown in Table 1.

TABLE 1
Experimental details of the study in Example 1

| | | | Concentration | Volume | | Test |
|---|---|---|---|---|---|---|
| Group | Treatment | N | (μg/mL) | (μL) | Route | time |
| 1 | Vehicle | 8 | 0 | 50 | s.c. (footpad) | 1 h |
| 2 | SEQ ID | 8 | 10/20 | 50 | s.c. (footpad) | 1 h |
| | NO: 29 | | | | | |

### Pain Measurements

Mechanical allodynia tests (also called “von Frey tests”) for pain measurements were performed on the rats one hour after administration of the test articles on Day 1 and Day 3, respectively. In the behavioral test for rats, mechanical allodynia is expressed as the 50% paw withdrawal threshold (PWT), which is calculated according to the following formula:

$$50 \% PWT ( g ) = ( 10 ⋀ ( Xf + k δ ) ) / 10 , 000$$

wherein
Xf=final test fiber value used in the test
k=tabular value for the pattern of positive/negative responses (Chaplan et al. 1994, page 62)
δ=mean difference.

Briefly, the rats were placed individually in plexiglass boxes with a grid at the bottom to ensure that the rats' feet could be tested. The rats were acclimated to the environment for 15 minutes prior to the test. After the acclimation was completed, pain (mechanical allodynia) was tested on the center of the sole of the left hind foot of the rats using test fibers. The test fibers included 8 test strengths: 3.61 (0.4 g), 3.84 (0.6 g), 4.08 (lg), 4.31 (2 g), 4.56 (4 g), 4.74 (6 g), 4.93 (8 g) and 5.18 (15 g). During the test, the test fibers were pressed vertically against the skin and forces were applied to bend the fibers for 6-8 seconds with a 5-second interval between each test. During the test, a pain response was recorded if the animals quickly withdrew their feet. The pain response was also recorded if the animal withdrew their feet as the test fibers left the animal's skin. The pain response was not recorded if the animal moved or ambulated, and the test should be repeated. The test was started with 4.31 (2 g). If the animal had pain response, the next test should be performed with a test fiber with a lower strength: if the animal has no pain response, the next test should be performed with a test fiber with a higher strength. The maximum strength of the tested fibers was 5.18 (15 g). The test results were recorded in a table, wherein the presence of pain response was recorded as “x”, and the absence of pain response was recorded as “0”. An exemplary table for use in the mechanical allodynia tests is shown in Table 2 hereinafter.

TABLE 2
An exemplary table for use in the mechanical allodynia tests

| | | | |
|---|---|---|---|
| 3.61 | | | |
| 3.84 | | | |
| 4.08 | | | |
| 4.31 | ∘ | | |
| 4.56 | ∘ | ∘ | ∘ |
| 4.74 | x | x | x |
| 4.93 | | | |
| 5.18 | | | |

### Results

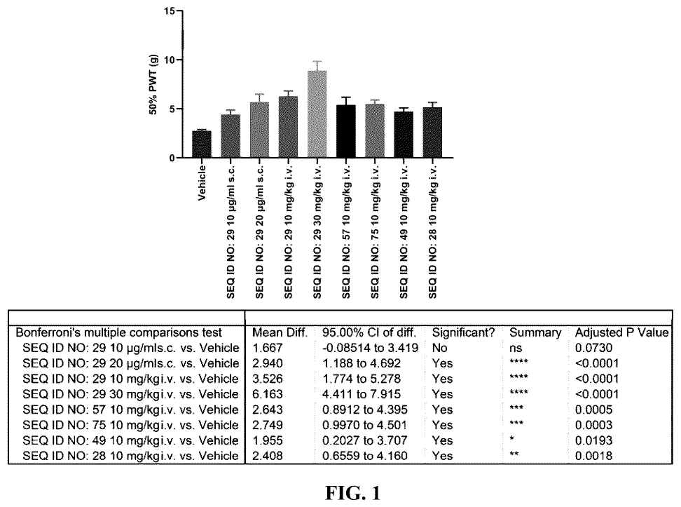

FIG. 1 shows the 50% PWT values of the rats from both placebo group and treatment groups (various concentrations of SEQ ID NO: 29, s.c., single administration) obtained from the mechanical allodynia tests of Example 1. It can be seen from FIG. 1 that the polypeptide of the present disclosure administered via s.c, route displayed apparent analgetic effect on VZV-induced pain model in rats: subcutaneous administration of the polypeptide SEQ ID NO: 29 increased the 50% PWT values of the rats in a dose-dependent manner.

### Example 2: Effect of the Polypeptide of the Present Disclosure Administered Via Intravenous Route on the Treatment of VZV-Induced Pain Model in Rats

In this Example, the effect of an exemplary polypeptide of the present disclosure, i.e. SEQ ID NO: 29 administered via intravenous (i.v.) route on the treatment of pain was investigated in VZV-induced pain model in rats.

### Modeling

The modeling was performed according to the method as recited in Example 1.

### Grouping and Administration

The rats were randomly divided into 2 groups, with 8 rats in the placebo group (Group 1, administered with vehicle (normal saline)) and 8 rats in the treatment group (Group 2, administered with 30 mg SEQ ID NO: 29/kg). Briefly, the test articles were administered to the rats on a single day by intravenous injection.

### Pain Measurements

Mechanical allodynia tests were performed on the rats 0.5 h after administration of the test articles according to the method as recited in Example 1, respectively.

### Results

FIG. 1 also shows the 50% PWT values of the rats from both placebo group and treatment group (30 mg SEQ ID NO: 29/kg, i.v., single administration) obtained from the mechanical allodynia tests of Example 2. It can be seen from FIG. 1 that the polypeptide of the present disclosure administered via i.v, route displayed apparent analgetic effect on VZV-induced pain model in rats: intravenous administration of the polypeptide SEQ ID NO: 29 significantly increased the 50% PWT values of the rats.

### Example 3: Effect of Various Polypeptides of the Present Disclosure Administered Via Intravenous Route on the Treatment of VZV-Induced Pain Model in Rats

In this Example, the effects of various polypeptides of the present disclosure administered via i.v, route on the treatment of pain were investigated in VZV-induced pain model in rats.

### Modeling

The modeling was performed according to the method as recited in Example 1.

### Grouping and Administration

The rats were randomly divided into 3 groups, with 8 rats in the placebo group (Group 1, administered with vehicle (normal saline)) and 16 rats in the treatment groups (Groups 2 and 3, 8 rats/group, administered with various polypeptides). Briefly, vehicle (normal saline) was administered to the rats from Group 1 and various polypeptides were administered to the rats from Group 2 (SEQ ID: 29, SEQ ID: 57 and SEQ ID: 49; 10 mg polypeptide/kg) and 3 (SEQ ID: 75 and SEQ ID: 28: 10 mg polypeptide/kg) on a single day by intravenous injection. More detailed information of the study is shown in Table 3.

TABLE 3
Experimental details of the study in Example 3

| | | | Dose | | |
|---|---|---|---|---|---|
| Group | Treatment | N | (mg/kg) | Route | Test time |
| 1 | Vehicle | 8 | 0 | i.v. | 0.5 h |
| 2 | Various polypeptides | 8 | 10 | i.v. | 0.5 h |
| 3 | Various polypeptides | 8 | 10 | i.v. | 0.5 h |

Note:
as for the animals in Groups 2 and 3, the administrations of different polypeptides were separated by a washout period (an approximate 2 to 3-day interval), respectively.

### Pain Measurements

Mechanical allodynia tests were performed on the rats 0.5 h after administration of the test articles according to the method as recited in Example 1, respectively.

### Results

FIG. 1 also shows the 50% PWT values of the rats from both placebo group and treatment groups (10 mg polypeptide/kg, i.v., single administration) obtained from the mechanical allodynia tests of Example 3. It can be seen from FIG. 1 that various polypeptides of the present disclosure, including SEQ ID NO: 28, SEQ ID NO: 29, SEQ ID NO: 49, SEQ ID NO: 57 and SEQ ID NO: 75 administered via i.v, route displayed apparent analgetic effect on VZV-induced pain model in rats.

### Example 4: Effect of Various Polypeptides of the Present Disclosure Administered Via Intravenous Route on the Treatment of HFD Plus STZ-Induced Pain Model in Mice

In this Example, the effects of various polypeptides of the present disclosure administered via i.v, route on the treatment of pain were investigated in high-fat diet (HFD) plus STZ (streptozocin)-induced pain model in mice.

### Modeling

A summary of the method for inducing pain model in mice by means of high-fat diet plus STZ injection is provided hereinafter. Briefly, C57BL/6 mice were fed with high-fat diet (HFD) for 4 weeks, STZ (100 mg/kg) was injected once intraperitoneally after 4 weeks of HFD feeding, followed by continuous HFD feeding for another 4 weeks. Fasting was required for 6 hours before STZ injection and 2 hours after STZ injection, respectively. Blood glucose test was performed 1 week after STZ injection (more than 12 hours of fasting was required before the test). Blood glucose test was performed 4 weeks after the injection of STZ, and then the mice were grouped.

### Grouping, Administration and Pain Measurements

Briefly, after modeling, 20 model mice in total were randomly divided into 2 groups (10 rats/group) according to their blood glucose levels. Various polypeptides of the present disclosure (SEQ ID NOs: 28, 29, 57, 86, 89, 90, 91, 93, 94, 95, 104, 106, 107, 108, 109 and 110) were administered to the mice (20 mg/kg, i.v., single administration) according to following protocol: Group 1: SEQ ID NO: 28, SEQ ID NO: 57, SEQ ID NO: 104, SEQ ID NO: 106, SEQ ID NO: 107, SEQ ID NO: 108, SEQ ID NO: 109 and SEQ ID NO: 110; and Group 2: SEQ ID NO: 29, SEQ ID NO: 86, SEQ ID NO: 89, SEQ ID NO: 90, SEQ ID NO: 91, SEQ ID NO: 93, SEQ ID NO: 94 and SEQ ID NO: 95. The mice were subjected to mechanical allodynia tests before and 1 h after administration according to a method similar to the one as recited in Example 1 but with slight modifications (the test strengths of the test fibers were 2.36 (0.02 g), 2.44 (0.04 g), 2.83 (0.07 g), 3.22 (0.16 g), 3.61 (0.4 g), 3.84 (0.6 g), 4.08 (1 g), 4.17 (1.4 g) instead, and the starting test strength was 3.22 (0.16 g)). More detailed information of the study is shown in Table 4.

TABLE 4
Experimental details of the study in Example 4

| | | | | | | Route |
|---|---|---|---|---|---|---|
| | | | | Dose | Fre- | (10 |
| Group | Model | Compound | N | (mg/kg) | quency | mL/kg) |
| 1 | HFD + STZ | Various | 10 | 20 | Once | i.v. |
| | | polypeptides | | | | |
| 2 | HFD + STZ | Various | 10 | 20 | Once | i.v. |
| | | polypeptides | | | | |

Note:
as for the animals in Groups 1 and 2, the administrations of different polypeptides were separated by a washout period (an approximate 3 to 5-day interval), respectively.

### Results

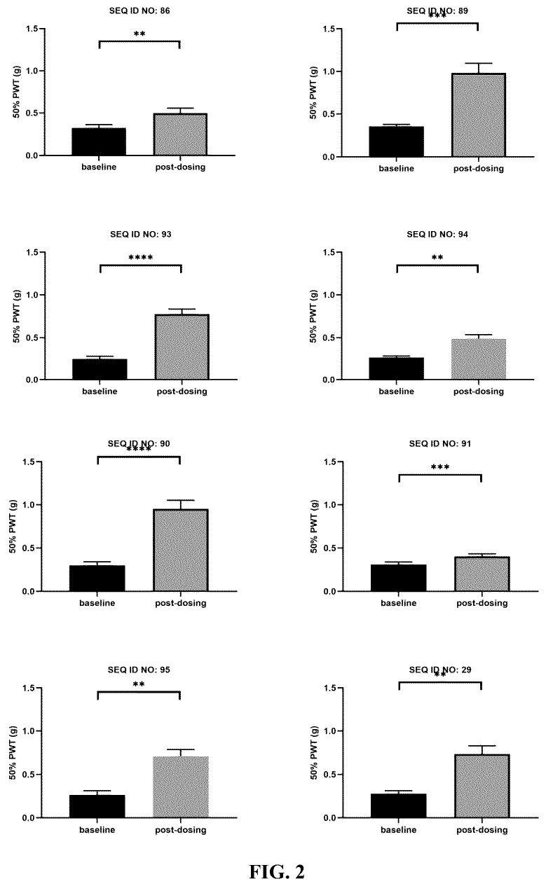

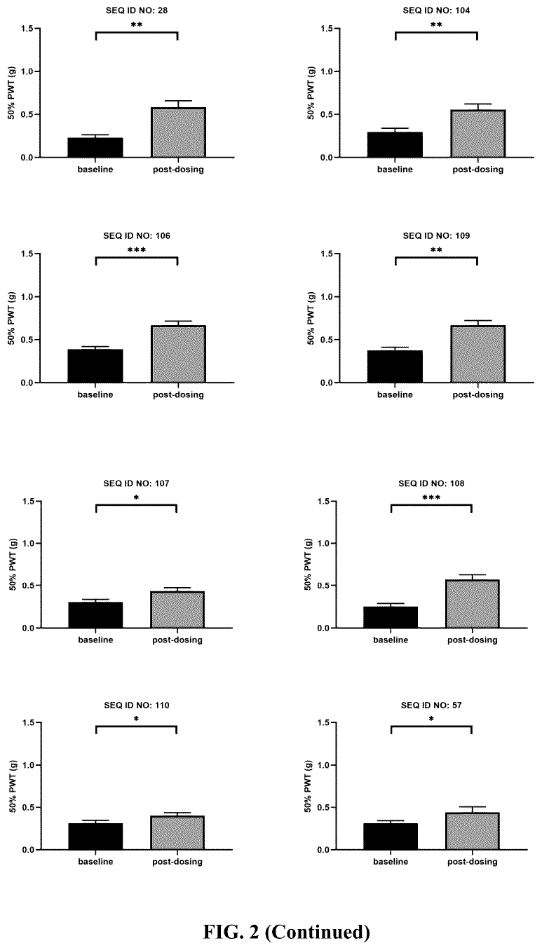

FIG. 2 shows the 50% PWT values of the mice from various treatment groups (20 mg polypeptide/kg, i.v., single administration) obtained from the mechanical allodynia tests of Example 4. It can be seen from FIG. 2 that various polypeptides of the present disclosure being tested in the study, including SEQ ID NO: 28, SEQ ID NO: 29, SEQ ID NO: 57, SEQ ID NO: 104, SEQ ID NO: 106, SEQ ID NO: 107, SEQ ID NO: 108, SEQ ID NO: 109, SEQ ID NO: 110, SEQ ID NO: 86, SEQ ID NO: 89, SEQ ID NO: 90, SEQ ID NO: 91, SEQ ID NO: 93, SEQ ID NO: 94 and SEQ ID NO: 95 displayed similar analgetic effect on HFD plus STZ-induced pain model in mice when administrated intravenously.

### Example 5: Effect of Various Polypeptides of the Present Disclosure Administered Via Intravenous Route on the Treatment of SNL Pain Model in Rats

In this Example, the effects of various polypeptides of the present disclosure on the treatment of pain were investigated in SNL (spinal nerve ligation) pain model in rats.

### Modeling

A summary of the method for inducing pain model in rats by means of spinal nerve ligation is provided hereinafter. Briefly, male SD rats were anesthetized with Zoletil R 50+xylazine hydrochloride (20 mg/kg+8 mg/kg, intraperitoneal injection). The surgical areas on the waist regions of the animals were shaved, and the skin was disinfected with iodophor and 70% v/v ethanol for three times. The left spinal nerves L5 and L6 of the animals were isolated, tightly ligated with 6-0 silk suture, and the wounds were sutured.

On the 11th day post operation, the animals were acclimated to the experimental environment, 15 minutes/day, for 3 consecutive days. On the 13th day post operation, the basal values of mechanical allodynia of the model rats were measured, and the animals were randomly divided into various groups after excluding those without exhibiting mechanical allodynia (with a 50% PWT value of greater than 5 g).

### Grouping, Administration and Pain Measurements

The study was divided into 2 parts, wherein a first group of 34 animals were used in part I of the study, and a second group of another 30 animals were used in part II of the study. The experiments of Part I and part II were performed separately.

Part I: evaluation of the effect of single intravenous administration of various doses of an exemplary polypeptide of the present disclosure, i.e. SEQ ID NO: 29 on the treatment of SNL pain model in rats

Part I of the study was performed in accordance with the experimental details shown in Table 5.

TABLE 5
Experimental details of Part I of the study in Example 5

| | | | Dose | | | | Test |
|---|---|---|---|---|---|---|---|
| Group | Model | Treatment | (mg/kg) | Formulation | Route | N | time |
| 1 | Sham | NA | NA | NA | NA | 10 | 1 h |
| 2 | SNL | Vehicle | NA | Saline | i.v. | 8 | 1 h |
| 3 | SNL | SEQ ID | 30 | Saline | i.v. | 8 | 1 h |
| | | NO: 29 | | | | | |
| 4 | SNL | SEQ ID | 60 | Saline | i.v. | 8 | 1 h |
| | | NO: 29 | | | | | |

Briefly, after SNL modeling, 24 animals were randomly divided into 3 groups (Groups 2, 3 and 4, N=8/group). Group 1 (a Sham group, for which the 10 animals received essentially the same operation procedure as other groups but their left spinal nerves L5 and L6 were neither isolated nor ligated) was used as the control group. The test articles were administrated to the animals according to the protocol shown in Table 5, and mechanical allodynia tests were respectively performed 1h after administration according to the method as recited in Example 1.

Part II: evaluation of the effects of single intravenous administration of various polypeptides of the present disclosure on the treatment of SNL pain model in rats

Part II of the study was performed in accordance with the experimental details shown in Table 6.

TABLE 6
Experimental details of Part II of the study in Example 5

| Group | Model | Treatment | Dose (mg/kg) | N | Route |
|---|---|---|---|---|---|
| 1 | SNL | Sham | NA | 6 | NA |
| 2 | SNL | SEQ ID NO: 90 | 60 | 12 | i.v. |
| 3 | SNL | SEQ ID NO: 93 | 60 | 12 | i.v. |

Briefly, after SNL modeling, 30 animals were randomly divided into 3 groups (Group 1, N=6/group; Groups 2 and 3, N=12/group). Various polypeptides of the present disclosure were administered to the animals from Groups 2 and 3, and the animals were subjected to mechanical allodynia tests 1 h after administration according to the method as recited in Example 1.

### Results

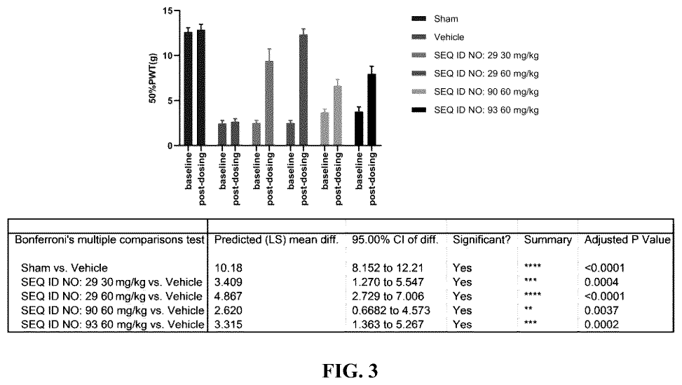

FIG. 3 shows the 50% PWT values of the mice from the Sham group, the vehicle group and various treatment groups (SEQ ID NOs: 29, 90 and 93, i.v., single administration) obtained from the mechanical allodynia tests of Example 5. It can be seen from FIG. 3 that various polypeptides of the present disclosure being tested in the study, including SEQ ID NO: 29, SEQ ID NO: 90 and SEQ ID NO: 93 have certain analgetic effect on SNL pain model in rats when administrated intravenously. Moreover, it is shown that the analgetic effect produced from the dose of 60 mg SEQ ID NO: 29/kg were much higher than that produced from the dose of 30 mg SEQ ID NO: 29/kg, suggesting a dose-dependent manner of the analgetic effect of the polypeptide of the present disclosure.

### Example 6: Effect of the Polypeptide of the Present Disclosure Administered Via Intravenous Route on the Treatment of Bone Pain Model Induced by Walker 256 Breast Cancer Cells in Rats

Bone metastasis is believed to be one of the most common causes of pain in cancer patients. Upon implantation in rats, Walker 256 cells may trigger significant bone resorption and increase skeletal fragility at the site of implantation (Kurth et al., (2000). Treatment with ibandronate preserves bone in experimental tumour-induced bone loss. J. Bone Joint Surg. Br. 82, 126-130.), being representative of the phenotype observed in breast cancer patients bearing bone metastasis (Shih et al., (2004). Bone resorption activity of osteolytic metastatic lung and breast cancers. J. Orthop. Res. 22, 1161-1167). Therefore, in this Example, the effect of an exemplary polypeptide of the present disclosure, i.e. SEQ ID NO: 29 administered via intravenous (i.v.) route on the treatment of pain was investigated in a bone pain model induced by Walker 256 breast cancer cells in rats.

### Grouping

28 female Wistar rats in total were randomly divided into 3 groups based on body weights, with 8 rats in the Sham group (Group 1, administered with vehicle (normal saline)), 10 rats in the model group (Group 2, administered with vehicle (normal saline)) and 10 rats in the treatment group (Group 3, administered with 60 mg SEQ ID NO: 29/kg).

### Modeling

A summary of the method for inducing bone pain model in rats by means of intratibial injection of a suspension of Walker 256 breast cancer cells is provided hereinafter. Briefly, the rats were anesthetized, then 4 μL of a suspension of Walker 256 cancer cells (containing about 4× 105 cells) was injected into the tibia cavity of the right hind limb of the rats (day 0). The rats in Sham group were injected with an equal volume of normal saline.

### Administration

After injection of cancer cells (day 0), administration was started on the next day (day 1) and performed once a day for a consecutive period of 14 days (day 1, day 2 . . , day 14, respectively). More detailed information of the study is shown in Table 7.

TABLE 7
Experimental details of the study in Example 6

| Group | Description | Treatment | Route | Dose (mg/kg) | N |
|---|---|---|---|---|---|
| 1 | Sham | Vehicle | i.v. | 0 | 8 |
| 2 | Model | Vehicle | i.v. | 0 | 10 |
| 3 | Treatment | SEQ ID NO: 29 | i.v. | 60 | 10 |

### Pain Measurements

The rats were subjected to mechanical allodynia tests 1 h before and after administration of the test articles according to the method as recited in Example 1 on day 7 (D7) and day 14 (D14), respectively.

### Results

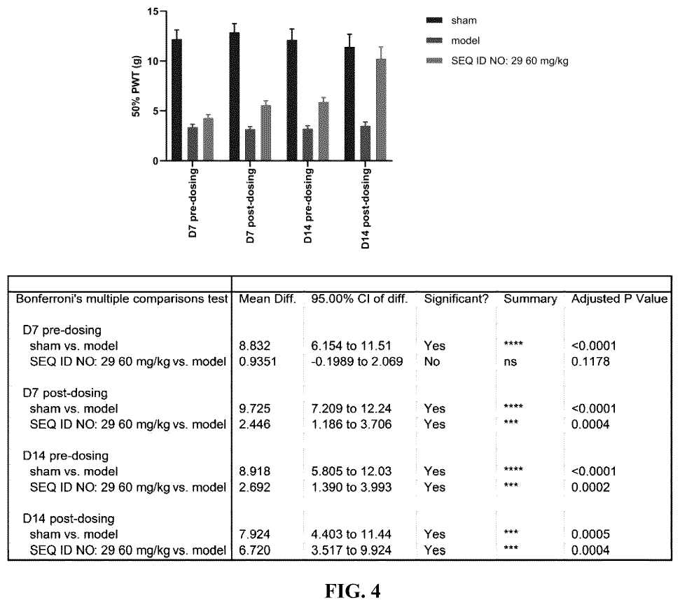

FIG. 4 shows the 50% PWT values of the rats from the Sham group, the model group and the treatment group (SEQ ID NO: 29, i.v., continuous administration) obtained from the mechanical allodynia tests of Example 6. It can be seen from FIG. 4 that before administration on day 7 (D7), compared with the Sham group, the pain thresholds of rats in the model group decreased significantly, and there was no significant difference between the pain thresholds of rats in the treatment group and those of rats in the model group: 1 h after administration on day 7 (D7), compared with the model group, the pain thresholds of rats in the treatment group increased significantly: before administration on day 14 (D14), compared with the model group, the pain thresholds of rats in the treatment group increased significantly; and 1 h after administration on day 14 (D14), compared with the model group, the pain thresholds of rats in the treatment group increased significantly. In summary, the data in FIG. 4 suggests that compared with the Sham group, the pain thresholds of rats in the model group decreased significantly, indicating that the modeling was successful. In addition, it is also shown that compared with the model group, the pain thresholds of rats in the treatment group increased gradually with the increase of the number of administrations, indicating a significant analgesic effect.

### Example 7: Effect of the Polypeptide of the Present Disclosure Administered Via Intraperitoneal Route on the Treatment of Cancer Pain Associated with Chemotherapy-Induced Peripheral Neuropathy Model Induced by Cisplatin in Mice

Administration of chemotherapeutic agents may cause damage to peripheral nerves, leading to chemotherapy-induced peripheral neuropathy (CIPN). Cancer pain associated with CIPN is one of the typical symptoms of CIPN. The pain may be described as a burning, tingling or pins-and-needles sensation, leading to difficulty of the patients in controlling fine motor skills. The pain is very common in cancer patients undergoing chemotherapy, especially those treated with cytotoxic agents (e.g, platinum anticancer agents, vinca alkaloids, taxanes, proteasome and angiogenesis inhibitors). Therefore, in this Example, the effect of an exemplary polypeptide of the present disclosure, i.e., SEQ ID NO: 29 administered via intraperitoneal (i.p.) route on the treatment of pain was investigated in a cancer pain associated with chemotherapy-induced peripheral neuropathy model induced by cisplatin in mice.

### General Experimental Protocol

The experiments in this Example were divided into two parts. In the first part (Part I), the administration of SEQ ID NO: 29 was performed in parallel with modeling to explore the preventive effect of the polypeptide of the present disclosure on cancer pain associated with CIPN, in particular CIPN induced by cisplatin. In the second part (Part II), after elution of SEQ ID NO: 29 from the mice's bodies, mechanical allodynia tests were performed on the mice to confirm that the animals still had the cancer pain associated with CIPN, and to explore the therapeutic effect of the polypeptide of the present disclosure on cancer pain associated with CIPN after modeling.

### Grouping and Administration

44 male C57BL/6 mice weighting 20 to 25 g were randomly divided into 4 groups, with 8 mice in the control group (Group 1, administered with vehicle (normal saline)), 12 mice in the model group (Group 2, administered with vehicle (normal saline) and cisplatin (2.3 mg/kg, i.p., qd)), 12 mice in the treatment group (qd) (Group 3, administered with SEQ ID NO: 29 (15 mg/kg, i.p., qd) and cisplatin (2.3 mg/kg, i.p., qd)) and 12 mice in the treatment group (bid) (Group 4, administered with SEQ ID NO: 29 (15 mg/kg, i.p., bid) and cisplatin (2.3 mg/kg, i.p., qd)).

Administration of SEQ ID NO: 29 was performed on scheduled dates. Briefly, SEQ ID NO: 29 was administered qd or bid by intraperitoneal injection on each day from Day 1 to Day 11, followed by a washing-out period ranging from Day 12 to Day 23. Afterwards, the administration of SEQ ID NO: 29 was resumed on the basis of the same regimen as above, lasting from Day 24 to Day 30.

More detailed information of the study is shown in Table 8.

TABLE 8
Experimental details of the study in Example 7

| | | | | | | |
|---|---|---|---|---|---|---|
| | | Com- | Dose | | | |
| Group | Model | pound | (mg/kg) | Route | Dosing regimen | N |

Part 1:

| | | | | | | |
|---|---|---|---|---|---|---|
| 1 | — | Saline | — | i.p. | Normal saline was | 8 |
| | | | | | administrated i.p. in | |
| | | | | | the morning, once a | |
| | | | | | day from Day 1 to | |
| | | | | | Day 11 | |
| 2 | CIPN | Saline | — | i.p. | Normal saline was | 12 |
| | | | | | administrated i.p. in | |
| | | | | | the morning, once a | |
| | | | | | day; 2 hours after | |
| | | | | | administration of | |
| | | | | | normal saline, 2.3 | |
| | | | | | mg/kg of cisplatin was | |
| | | | | | administrated i.p.; | |
| | | | | | normal saline was | |
| | | | | | administrated from | |
| | | | | | Day 1 to Day 11, and | |
| | | | | | cisplatin was | |
| | | | | | administrated from | |
| | | | | | Day 1 to Day 5 and | |
| | | | | | Day 11 | |
| 3 | CIPN | SEQ ID | 15 | i.p. | SEQ ID NO: 29 was | 12 |
| | | NO: 29 | | | administrated i.p. in | |
| | | | | | the morning, once a | |
| | | | | | day; 2 hours after | |
| | | | | | administration of SEQ | |
| | | | | | ID NO: 29, 2.3 mg/kg | |
| | | | | | of cisplatin was | |
| | | | | | administrated i.p.; | |
| | | | | | SEQ ID NO: 29 was | |
| | | | | | administrated from | |
| | | | | | Day 1 to Day 11, and | |
| | | | | | cisplatin was | |
| | | | | | administrated from | |
| | | | | | Day 1 to Day 5 and | |
| | | | | | Day 11 | |
| 4 | CIPN | SEQ ID | 15 | i.p. | SEQ ID NO: 29 was | 12 |
| | | NO: 29 | | | administrated i.p. in | |
| | | | | | the morning for the | |
| | | | | | first time; 2 hours | |
| | | | | | after the first | |
| | | | | | administration of SEQ | |
| | | | | | ID NO: 29, 2.3 mg/kg | |
| | | | | | of cisplatin was | |
| | | | | | administrated i.p.; and | |
| | | | | | 6 hours after the first | |
| | | | | | administration of SEQ | |
| | | | | | ID NO: 29, SEQ ID | |
| | | | | | NO: 29 was | |
| | | | | | administrated i.p. | |
| | | | | | again (i.e., SEQ ID | |
| | | | | | NO: 29 was | |
| | | | | | administrated twice a | |
| | | | | | day); SEQ ID NO: 29 | |
| | | | | | was administrated | |
| | | | | | from Day 1 to Day 11, | |
| | | | | | and cisplatin was | |
| | | | | | administrated from | |
| | | | | | Day 1 to Day 5 and | |
| | | | | | Day 11 | |

Part 2:

| | | | | | | |
|---|---|---|---|---|---|---|
| 1 | — | Saline | — | i.p. | Normal saline was | 8 |
| | | | | | administrated i.p. in | |
| | | | | | the morning, once a | |
| | | | | | day, from Day 24 to | |
| | | | | | Day 30 | |
| 2 | CIPN | Saline | — | i.p. | Normal saline was | 12 |
| | | | | | administrated i.p. in | |
| | | | | | the morning, once a | |
| | | | | | day, from Day 24 to | |
| | | | | | Day 30 | |
| 3 | CIPN | SEQ ID | 15 | i.p. | SEQ ID NO: 29 was | 12 |
| | | NO: 29 | | | administrated i.p. in | |
| | | | | | the morning, once a | |
| | | | | | day from Day 24 to | |
| | | | | | Day 30 | |
| 4 | CIPN | SEQ ID | 15 | i.p. | SEQ ID NO: 29 was | 12 |
| | | NO: 29 | | | administrated i.p. in | |
| | | | | | the morning for the | |
| | | | | | first time; 6 hours | |
| | | | | | after the first | |
| | | | | | administration of SEQ | |
| | | | | | ID NO: 29, SEQ ID | |
| | | | | | NO: 29 was | |
| | | | | | administrated i.p. | |
| | | | | | again (i.e., SEQ ID | |
| | | | | | NO: 29 was | |
| | | | | | administrated twice a | |
| | | | | | day), from Day 24 to | |
| | | | | | Day 30; | |

### Pain Measurements

The mice were subjected to mechanical allodynia tests 0 h before administration and 2 h after the first administration of the test articles (normal saline or SEQ ID NO: 29) according to the method as recited in Example 1 on Day 6, Day 10, Day 23 and Day 30, respectively.

### Results

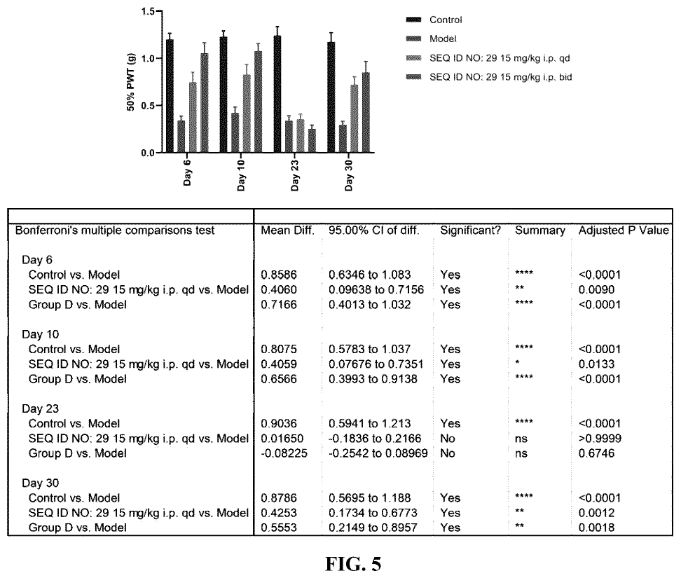

FIG. 5 shows the 50% PWT values of the mice from the control group, the model group, the treatment group (qd) (SEQ ID NO: 29, i.p., qd) and the treatment group (bid) (SEQ ID NO: 29, i.p., bid) obtained from the mechanical allodynia tests of Example 7. It can be seen from FIG. 5 that the polypeptide of the present disclosure has a preventive effect on cancer pain associated with CIPN. Specifically, the results of pain measurements on Day 6 and Day 10 showed that the pain thresholds of the mice from the treatment groups (close to those of the mice from the control group) were significantly higher than those of the mice from the model group. It was also found that the levels of pain thresholds of the mice were different under different dosing frequency (i.e., were dose-related). It can also be seen from FIG. 5 that the polypeptide of the present disclosure has a therapeutic effect on cancer pain associated with CIPN. In particular, the results of pain measurements on Day 30 showed that the pain thresholds of the mice from the treatment groups were significantly increased after 7 days of continuous administration of SEQ ID NO: 29 (see the changes in the pain thresholds of the mice from the treatment groups on Day 30 vs Day 23).

### Example 8: Effects of the Polypeptide of the Present Disclosure Administered Via Ophthalmic Route on the Treatment of Eye Pain Model Induced by Benzalkonium Chloride in Mice

Eye pain, also called pain in the eye, may be described as a sharp, aching or throbbing in one eye or both eyes. Eye pain can be largely classified as ocular nociceptive pain and ocular neuropathic pain. Ocular nociceptive pain is often caused by the various insults present at the front of the eye, such as injury, surgery, contact lenses and foreign bodies. In contrast, ocular neuropathic pain refers to the heightened perception of eye pain in response to normally non-painful stimuli. This condition might arise from repeated direct damage to corneal nerves, wherein aberrant regeneration of corneal nerves and upregulation of nociceptors which are responsible for processing of painful stimuli bring about increased perception of pain in response to even normally unpainful stimuli. In this Example, the effect of an exemplary polypeptide of the present disclosure, i.e. SEQ ID NO: 29 administered via ophthalmic route (as eye drops) on the treatment of pain was investigated in an eye pain (ocular neuropathic pain) model induced by benzalkonium chloride in mice.

### Modeling

C57BL/6JNifdc mice were used in the experiment. Briefly, each of the mice was fixed with one hand, with one eye prop opened with the other hand (and fixed with another lab technician). The modeling drug, i.e., 0.1% benzalkonium chloride was dripped into one eye (twice a day (9:00, 17:00), 10 μL each time) and maintained for 30 seconds. The operation was repeated for six days.

In order to evaluate the pathological changes at the front of the mice's eyes, slit lamp photos were taken on the third day since the beginning of modeling. According to the photographic results, the mice's eyes were scored from three viewing angles, including corneal opacity (0: clear, 1: visible opacity, 2: difficult to find, 3: pupil invisible), conjunctival edema (0: absent, 1: slight edema, 2: edema, 3: severe edema) and congestion (0: absent, 1: slight congestion, 2: congestion, 3: severe congestion, 4: hemorrhage), to access and evaluate the damages at the front of the mice's eyes. The mice were then randomized into groups.

### Grouping and Administration

18 model mice were randomly divided into 2 groups based on their scores in the front-eye evaluation, with 9 mice in the model group and 9 mice in the test substance group. Another 9 mice that had not undergone modeling constituted the control group.

For the test substance group, the polypeptide of the present disclosure was administered according to the following regimen. Specifically, each of the mice was fixed with one hand, with the modelling eye prop opened with the other hand. The test substance (SEQ ID NO: 29) was dripped into the modelling eye with a pipette (3 times a day (9:00, 13:00, 17:00), 10 μL each time, at a concentration of 30 μg/ml) and maintained for 30 seconds. The operation was repeated for two days. A scratching behavior test was then performed to investigate the analgesic effect of the test substance on eye pain of the mice.

More detailed information of grouping and administration is shown in Table 9.

TABLE 9
Experimental details of the study in Example 8

| Group | Description | N | Route | Dose |
|---|---|---|---|---|
| 1 | Control | 9 | \ | \ |
| 2 | Model | 9 | \ | \ |
| 3 | Model + compound | 9 | Eye drops, TID | 0.3 μg/eye/each time |
| | (test substance) | | | |

### Pain Measurements

Scratching behavior test: The eyes were administered for two consecutive days. After the first administration on the morning of the third day, the mice were released into the test cage to accommodate to the environment. After the mice had accommodated for 20 minutes, 2M NaCl hypertonic saline was dripped into their eyes (7 μL/mouse). After acting for 5 seconds, the mice were released back into the cage, and the numbers of wipes over the eyes within 30 seconds were counted.

### Results

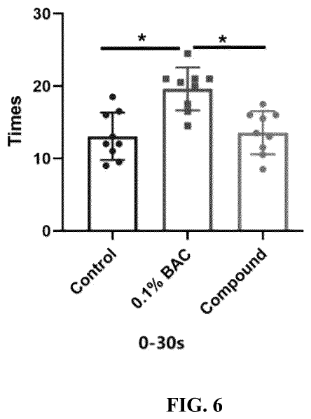

FIG. 6 shows the numbers of wipes over the eyes of the mice from respective groups in the scratching behavior test. During the modeling process, the cornea and conjunctiva of the mice were damaged due to consecutive administration of 0.1% benzalkonium chloride, causing symptoms such as eye pain and itching. Therefore, the scratching behavior test was performed to observe the occasions of wiping over the eyes with the front paws of the mice after 2 days of treatment. It was found that, the mice in the model group exhibited significantly increased numbers of wiping over the eyes (P<0.05) compared with the mice in the control group; in contrast, the mice in test substance group exhibited significantly decreased numbers of wiping over the eyes (P<0.01) compared with the mice in the model group after administration of the test substance. The results showed that the test substance can significantly improve the eye pain symptoms of the mice.

1.-52. (canceled)

53. A method for treating or preventing pain in a subject in need thereof, the method comprising:
administering to the subject an effective amount of an artificial polypeptide of formula (I): 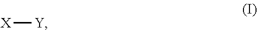
wherein:
X is a moiety comprising an amino acid sequence having at least 80% sequence identity with SEQ ID NO: 1, and
Y is a moiety comprising 10 to 50 amino acids, at least 50% of which are selected from the group consisting of: isoleucine (I), valine (V), leucine (L), phenylalanine (F), cysteine (C), methionine (M), and alanine (A); and
wherein Y comprises a total number of C of less than 5.

54. The method of claim 53, wherein X comprises an amino acid sequence having at least 90% sequence identity with SEQ ID NO: 1.

55. The method of claim 53, wherein at least 50% amino acids of X are selected from the group consisting of: arginine (R), lysine (K), asparagine (N), aspartic acid (D), glutamine (Q), glutamic acid (E), and histidine (H).

56. The method of claim 53, wherein Y comprises a total number of C of less than 4.

57. The method of claim 53, wherein a total number of hydrophobic amino acids in Y is more than 8 and/or a total number of hydrophilic amino acids in Y is no more than 5.

58. The method of claim 53, wherein X comprises an amino acid sequence having at least 90% sequence identity with any one of SEQ ID NOs: 81-83.

59. The method of claim 53, wherein Y comprises an amino acid sequence having at least 90% sequence identity with any one of SEQ ID NOs: 3-18, 58-61, 84, or 85.

60. The method of claim 53, wherein the artificial polypeptide comprises an amino acid sequence having at least 90% sequence identity with any one of SEQ ID NOs: 29-41, 54-57, 75, or 91-95.

61.-90. (canceled)

91. The method of claim 53, wherein the pain is at least one selected from the group consisting of: acute pain, chronic pain, and transitional pain.

92. The method of claim 91, wherein the acute pain is at least one selected from the group consisting of pain associated with acute injuries, acute inflammatory pain, and headache; or
wherein the chronic pain is at least one selected from the group consisting of: nociceptive pain, neuropathic pain, and mixed pain.

93. The method of claim 92, wherein the pain associated with acute injuries is acute postoperative pain;
wherein the nociceptive pain is at least one selected from the group consisting of:
abdominal pain, anal fissure pain, bladder pain, complex regional pain syndrome, mastalgia, enterospasm, bladder pain syndrome, arthralgia, musculoskeletal pain, muscle pain, myofascial pain syndrome, nociceptive bone pain, pain associated with pancreatitis, polymyalgia rheumatica, chronic postoperative pain, renal pain, somatic pain, tenodynia, desmalgia, chronic traumatic pain, pain associated with fracture, and visceral pain;
wherein the mixed pain is at least one selected from the group consisting of;
arthritis pain, back pain, cancer pain, toothache, fibromyalgia, chronic inflammatory pain, lumbago, cervicodynia, and eye pain; or
wherein the neuropathic pain is at least one selected from the group consisting of: central neuropathic pain and peripheral neuropathic pain.

94. (canceled)

95. (canceled)

96. The method of claim 93, wherein the mastalgia is cyclic mastalgia;
wherein the renal pain is renal colic;
wherein the pain associated with fracture is pain associated with spine fracture;
wherein the arthritis pain is at least one selected from the group consisting of: osteoarthritis pain, rheumatoid arthritis pain, and gout pain;
wherein the cancer pain is at least one selected from the group consisting of: cancer pain associated with tumor, cancer pain associated with diagnostic procedures, and cancer pain associated with therapeutic procedures;
wherein the eye pain is at least one selected from the group consisting of: ocular nociceptive pain and ocular neuropathic pain;
wherein the central neuropathic pain is at least one selected from the group consisting of: post-stroke neuropathic pain, syringomyelia pain, neuropathic pain associated with ischemic myelopathy, neuropathic pain associated with oppressed myelopathy, neuropathic pain associated with radiation myelopathy, neuropathic pain associated with spinal cord injury, neuropathic pain associated with multiple sclerosis, neuropathic pain associated with Parkinson's disease, phantom limb pain, and neuropathic pain associated with myelitis; or
wherein the peripheral neuropathic pain is at least one selected from the group consisting of: peripheral neuropathic pain associated with metabolic disorder or ischemia, peripheral neuropathic pain associated with infection, peripheral neuropathic pain associated with nerve or in nerve root compression, peripheral neuropathic pain associated with chemotherapy, peripheral neuropathic pain associated with radiotherapy, stump pain, peripheral neuropathic pain associated with neuropathy due to tumor compression or infiltration, peripheral neuropathic pain associated with alcoholic polyneuropathy, peripheral neuropathic pain associated with dystrophic neuropathy, peripheral neuropathic pain associated with toxic peripheral neuropathy, and peripheral neuropathic pain associated with immune neuropathy.

97.-101. (canceled)

102. The method of claim 96,
wherein the cancer pain associated with tumor is at least one selected from the group consisting of: nerve pain associated with cancer, bone pain associated with cancer, soft tissue pain associated with cancer, and referred pain associated with cancer;
wherein the cancer pain associated with therapeutic procedures is at least one selected from the group consisting of: phantom pain associated with cancer and cancer pain associated with chemotherapy;
wherein the ocular nociceptive pain is at least one selected from the group consisting of: eye pain associated with injury, eye pain associated with surgery, eye pain associated with contact lenses wearing, and eye pain associated with irritation of foreign bodies;
wherein the ocular neuropathic pain is at least one selected from the group consisting of: eye pain associated with allergies, eye pain associated with infection, eye pain associated with inflammation, eye pain associated with chronic ocular surface disease, post-surgical ocular neuropathic pain, eye pain associated with toxic keratopathy, eye pain associated with radiation, eye pain associated with ultraviolet light exposure, eye pain associated with a systemic neuropathy, traumatic ocular neuropathic pain, eye pain with trigeminal neuralgia, eye pain associated with fibromyalgia, corneal neuralgia, conjunctival neuralgia, optic nerve neuralgia, extraocular muscle neuralgia, orbital neuralgia, and eyelid neuralgia;
wherein the peripheral neuropathic pain associated with metabolic disorder or ischemia is at least one selected from the group consisting of: diabetic peripheral neuropathic pain and ischemic peripheral neuropathic pain;
wherein the peripheral neuropathic pain associated with infection is at least one selected from the group consisting of: peripheral neuropathic pain associated with viral infection and peripheral neuropathic pain associated with spirochetal infection; or
wherein the peripheral neuropathic pain associated with nerve or nerve root compression is at least one selected from the group consisting of: sciatica, trigeminal neuralgia, glossopharyngeal neuralgia, neuropathic pain associated with radiculoneuropathy, and neuropathic pain associated with entrapment neuropathy.

103. The method of claim 102, wherein the bone pain associated with cancer is at least one selected from the group consisting of: bone pain associated with bone cancers, and bone pain associated with bone metastatic tumors;
wherein the cancer pain associated with chemotherapy is cancer pain associated with chemotherapy-induced peripheral neuropathy;
wherein the peripheral neuropathic pain associated with viral infection is at least one selected from the group consisting of: post-herpetic neuralgia and peripheral neuropathic pain associated with HIV infection; or
wherein the peripheral neuropathic pain associated with spirochetal infection is peripheral neuropathic pain associated with syphilis infection.

104. The method of claim 103, wherein the bone pain associated with bone cancers is at least one selected from the group consisting of: bone pain associated with chondrosarcoma, bone pain associated with Ewing's sarcoma, bone pain associated with malignant fibrous histiocytoma of bone, bone pain associated with osteosarcoma, and bone pain associated with fibrocartilaginous mesenchymoma of bone.

105.-108. (canceled)

109. The method of claim 102, wherein the cancer pain associated with chemotherapy is cancer pain associated with administration of a cytotoxic agent to the subject.

110. The method of claim 109, wherein the cytotoxic agent is at least one selected from the group consisting of: a platinum anticancer agent, a vinca alkaloid, a taxane, and a proteasome or angiogenesis inhibitor.

111.-130. (canceled)

131. The method of claim 53, wherein the subject is a mammal.

132. The method of claim 53, wherein the subject is a pet.

133. The method of claim 53, wherein the artificial polypeptide is administered in the form of a formulation comprising the artificial polypeptide and a pharmaceutically acceptable excipient.

134. The method of claim 133, wherein the formulation is in a form suitable for oral administration.

135. The method of claim 133, wherein the formulation is in a form suitable for parenteral administration.

136. The method of claim 133, wherein the formulation is in a form suitable for topical administration.

137. The method of claim 133, wherein the formulation is in a form of sustained, controlled, or delayed release formulation.

138. The method of claim 133, wherein the formulation is a formulation for use in human.

139. The method of claim 133, wherein the formulation is a veterinary formulation.


SEQUENCE LISTING (110 sequences)

SEQ ID NO: 1 - protein, 57 aa, synthetic construct
SEKKQPVDLGLLEEDDEFEEFPAEDWAGLDEDEDAHVWEDNWDDDNVEDDFSNQLRA

SEQ ID NO: 2 - protein, 31 aa, synthetic construct
TVLLMILVCETPYGCYVLHQKGRMCSAFLCC

SEQ ID NO: 3 - protein, 31 aa, synthetic construct
TVLLMILVAETPYGAYVLHQKGRMASAFLAA

SEQ ID NO: 4 - protein, 31 aa, synthetic construct
TVLLMILVCETPYGCYVLHQKGRMCSAFLCA

SEQ ID NO: 5 - protein, 31 aa, synthetic construct
TVLLMILVVETPYGVYVLHQKGRMVSAFLVV

SEQ ID NO: 6 - protein, 31 aa, synthetic construct
TVLLMILVAETPYGAYVLHQKGRMVSAFLVV

SEQ ID NO: 7 - protein, 31 aa, synthetic construct
TVLLMILVAETPYGVYVLHQKGRMASAFLVV

SEQ ID NO: 8 - protein, 31 aa, synthetic construct
TVLLMILVAETPYGVYVLHQKGRMVSAFLAV

SEQ ID NO: 9 - protein, 30 aa, synthetic construct
VLLMILVAETPYGVYVLHQKGRMVSAFLVA

SEQ ID NO: 10 - protein, 31 aa, synthetic construct
TVLLMILVVETPYGAYVLHQKGRMASAFLVV

SEQ ID NO: 11 - protein, 31 aa, synthetic construct
TVLLMILVVETPYGAYVLHQKGRMVSAFLAV

SEQ ID NO: 12 - protein, 31 aa, synthetic construct
TVLLMILVVETPYGAYVLHQKGRMVSAFLVA

SEQ ID NO: 13 - protein, 31 aa, synthetic construct
TVLLMILVVETPYGVYVLHQKGRMASAFLAV

SEQ ID NO: 14 - protein, 31 aa, synthetic construct; identical to SEQ ID NO: 13

SEQ ID NO: 15 - protein, 31 aa, synthetic construct
TVLLMILVVETPYGVYVLHQKGRMVSAFLAA

SEQ ID NO: 16 - protein, 31 aa, synthetic construct
TVLLVILVAETPYGAYVLHQKGRVASAFLAA

SEQ ID NO: 17 - protein, 31 aa, synthetic construct
GLCALCLMVRQPCFCLEIYMGVKHTCLYVST

SEQ ID NO: 18 - protein, 31 aa, synthetic construct
SLLCVFPTIECCRYKMGLLYHCVACTQGMLV

SEQ ID NO: 19 - protein, 57 aa, synthetic construct
SEFEDANKEQGEVPNDDVSDELPKLLDDDEDELRGALFFQWHDWEEAEDNDWDVEDA

SEQ ID NO: 20 - protein, 57 aa, synthetic construct
SELDFWDNWKDRQADLPAASDDEDFENDLAEVDHEDPLVDGNEWDGDEVEFELEEQK

SEQ ID NO: 21 - protein, 57 aa, synthetic construct
SDADEFDDDNSVWGWEDDEVEPNDAFQPLGDVRANEELDEKDHKLDEEADLQWEFEL

SEQ ID NO: 22 - protein, 57 aa, synthetic construct
SQWAPLDHLESDEWGAFEDEDFNNGDVEELDPEERDDEDLFQKDVLVAKADDEEWDN

SEQ ID NO: 23 - protein, 57 aa, synthetic construct
STKKQPVDLGLLETDSEFTEFPATSWAGLSESESAHVWTSNWDSSNVTSDFSNQLRA

SEQ ID NO: 24 - protein, 57 aa, synthetic construct
STKKQPVDLGLLETDDEFEEFPATSWAGLSEDESAHVWESNWDDSNVTSDFSNQLRA

SEQ ID NO: 25 - protein, 57 aa, synthetic construct
STKKQPVDLGLLEEDDEFEEFPAESWAGLDEDESAHVWEDNWDDDNVTSDFSNQLRA

SEQ ID NO: 26 - protein, 57 aa, synthetic construct
SEKKQPVDLGLLEEDDEFEEFPAEDWAGLDEDESAHVWEDNWDDDNVTSDFSNQLRA

SEQ ID NO: 27 - protein, 57 aa, synthetic construct
SEKKQPVDLGLLEEDDEFEEFPAEDWAGLDEDEDAHVWEDNWDDDNVTSDFSNQLRA

SEQ ID NO: 28 - protein, 88 aa, synthetic construct
SEKKQPVDLGLLEEDDEFEEFPAEDWAGLDEDEDAHVWEDNWDDDNVEDDFSNQLRATVLLMILVCETPYGCYVLHQKGRMCSAFLCC

SEQ ID NO: 29 - protein, 88 aa, synthetic construct
SEKKQPVDLGLLEEDDEFEEFPAEDWAGLDEDEDAHVWEDNWDDDNVEDDFSNQLRATVLLMILVAETPYGAYVLHQKGRMASAFLAA

SEQ ID NO: 30 - protein, 88 aa, synthetic construct
SEKKQPVDLGLLEEDDEFEEFPAEDWAGLDEDEDAHVWEDNWDDDNVEDDFSNQLRATVLLMILVCETPYGCYVLHQKGRMCSAFLCA

SEQ ID NO: 31 - protein, 88 aa, synthetic construct
SEKKQPVDLGLLEEDDEFEEFPAEDWAGLDEDEDAHVWEDNWDDDNVEDDFSNQLRATVLLMILVVETPYGVYVLHQKGRMVSAFLVV

SEQ ID NO: 32 - protein, 88 aa, synthetic construct
SEKKQPVDLGLLEEDDEFEEFPAEDWAGLDEDEDAHVWEDNWDDDNVEDDFSNQLRATVLLMILVAETPYGAYVLHQKGRMVSAFLVV

SEQ ID NO: 33 - protein, 88 aa, synthetic construct
SEKKQPVDLGLLEEDDEFEEFPAEDWAGLDEDEDAHVWEDNWDDDNVEDDFSNQLRATVLLMILVAETPYGVYVLHQKGRMASAFLVV

SEQ ID NO: 34 - protein, 88 aa, synthetic construct
SEKKQPVDLGLLEEDDEFEEFPAEDWAGLDEDEDAHVWEDNWDDDNVEDDFSNQLRATVLLMILVAETPYGVYVLHQKGRMVSAFLAV

SEQ ID NO: 35 - protein, 88 aa, synthetic construct
SEKKQPVDLGLLEEDDEFEEFPAEDWAGLDEDEDAHVWEDNWDDDNVEDDFSNQLRATVLLMILVAETPYGVYVLHQKGRMVSAFLVA

SEQ ID NO: 36 - protein, 88 aa, synthetic construct
SEKKQPVDLGLLEEDDEFEEFPAEDWAGLDEDEDAHVWEDNWDDDNVEDDFSNQLRATVLLMILVVETPYGAYVLHQKGRMASAFLVV

SEQ ID NO: 37 - protein, 88 aa, synthetic construct
SEKKQPVDLGLLEEDDEFEEFPAEDWAGLDEDEDAHVWEDNWDDDNVEDDFSNQLRATVLLMILVVETPYGAYVLHQKGRMVSAFLAV

SEQ ID NO: 38 - protein, 88 aa, synthetic construct
SEKKQPVDLGLLEEDDEFEEFPAEDWAGLDEDEDAHVWEDNWDDDNVEDDFSNQLRATVLLMILVVETPYGAYVLHQKGRMVSAFLVA

SEQ ID NO: 39 - protein, 88 aa, synthetic construct
SEKKQPVDLGLLEEDDEFEEFPAEDWAGLDEDEDAHVWEDNWDDDNVEDDFSNQLRATVLLMILVVETPYGVYVLHQKGRMASAFLAV

SEQ ID NO: 40 - protein, 88 aa, synthetic construct
SEKKQPVDLGLLEEDDEFEEFPAEDWAGLDEDEDAHVWEDNWDDDNVEDDFSNQLRATVLLMILVVETPYGVYVLHQKGRMASAFLVA

SEQ ID NO: 41 - protein, 88 aa, synthetic construct
SEKKQPVDLGLLEEDDEFEEFPAEDWAGLDEDEDAHVWEDNWDDDNVEDDFSNQLRATVLLMILVVETPYGVYVLHQKGRMVSAFLAA

SEQ ID NO: 42 - protein, 88 aa, synthetic construct
SEKKQPVDLGLLEEDDEFEEFPAEDWAGLDEDEDAHVWEDNWDDDNVEDDFSNQLRAGLCALCLMVRQPCFCLEIYMGVKHTCLYVST

SEQ ID NO: 43 - protein, 88 aa, synthetic construct
SEKKQPVDLGLLEEDDEFEEFPAEDWAGLDEDEDAHVWEDNWDDDNVEDDFSNQLRASLLCVFPTIECCRYKMGLLYHCVACTQGMLV

SEQ ID NO: 44 - protein, 88 aa, synthetic construct
SEKKQPVDLGLLEEDDEFEEFPAEDWAGLDEDEDAHVWEDNWDDDNVEDDFSNQLRATVLLVILVAETPYGAYVLHQKGRVASAFLAA

SEQ ID NO: 45 - protein, 88 aa, synthetic construct
SEFEDANKEQGEVPNDDVSDELPKLLDDDEDELRGALFFQWHDWEEAEDNDWDVEDATVLLMILVCETPYGCYVLHQKGRMCSAFLCC

SEQ ID NO: 46 - protein, 88 aa, synthetic construct
SELDFWDNWKDRQADLPAASDDEDFENDLAEVDHEDPLVDGNEWDGDEVEFELEEQKTVLLMILVCETPYGCYVLHQKGRMCSAFLCC

SEQ ID NO: 47 - protein, 88 aa, synthetic construct
SDADEFDDDNSVWGWEDDEVEPNDAFQPLGDVRANEELDEKDHKLDEEADLQWEFELTVLLMILVCETPYGCYVLHQKGRMCSAFLCC

SEQ ID NO: 48 - protein, 88 aa, synthetic construct
SQWAPLDHLESDEWGAFEDEDFNNGDVEELDPEERDDEDLFQKDVLVAKADDEEWDNTVLLMILVCETPYGCYVLHQKGRMCSAFLCC

SEQ ID NO: 49 - protein, 88 aa, synthetic construct
STKKQPVDLGLLETDSEFTEFPATSWAGLSESESAHVWTSNWDSSNVTSDFSNQLRATVLLMILVCETPYGCYVLHQKGRMCSAFLCC

SEQ ID NO: 50 - protein, 88 aa, synthetic construct
STKKQPVDLGLLETDDEFEEFPATSWAGLSEDESAHVWESNWDDSNVTSDFSNQLRATVLLMILVCETPYGCYVLHQKGRMCSAFLCC

SEQ ID NO: 51 - protein, 88 aa, synthetic construct
STKKQPVDLGLLEEDDEFEEFPAESWAGLDEDESAHVWEDNWDDDNVTSDFSNQLRATVLLMILVCETPYGCYVLHQKGRMCSAFLCC

SEQ ID NO: 52 - protein, 88 aa, synthetic construct
SEKKQPVDLGLLEEDDEFEEFPAEDWAGLDEDESAHVWEDNWDDDNVTSDFSNQLRATVLLMILVCETPYGCYVLHQKGRMCSAFLCC

SEQ ID NO: 53 - protein, 88 aa, synthetic construct
SEKKQPVDLGLLEEDDEFEEFPAEDWAGLDEDEDAHVWEDNWDDDNVTSDFSNQLRATVLLMILVCETPYGCYVLHQKGRMCSAFLCC

SEQ ID NO: 54 - protein, 83 aa, synthetic construct
SEKKQPVDLGLLEEDDEFEEFPAEDWAGLDEDEDAHVWEDNWDDDNVTSDFSNQLRATVLLMILVCETPYGCYVLHQKGRMCS

SEQ ID NO: 55 - protein, 78 aa, synthetic construct
SEKKQPVDLGLLEEDDEFEEFPAEDWAGLDEDEDAHVWEDNWDDDNVTSDFSNQLRATVLLMILVCETPYGCYVLHQK

SEQ ID NO: 56 - protein, 68 aa, synthetic construct
SEKKQPVDLGLLEEDDEFEEFPAEDWAGLDEDEDAHVWEDNWDDDNVTSDFSNQLRATVLLMILVCET

SEQ ID NO: 57 - protein, 63 aa, synthetic construct
SEKKQPVDLGLLEEDDEFEEFPAEDWAGLDEDEDAHVWEDNWDDDNVTSDFSNQLRATVLLMI

SEQ ID NO: 58 - protein, 57 aa, synthetic construct; identical to SEQ ID NO: 27

SEQ ID NO: 59 - protein, 57 aa, synthetic construct; identical to SEQ ID NO: 27

SEQ ID NO: 60 - protein, 57 aa, synthetic construct; identical to SEQ ID NO: 27

SEQ ID NO: 61 - protein, 57 aa, synthetic construct; identical to SEQ ID NO: 27

SEQ ID NO: 62 - protein, 69 aa, synthetic construct
SEKKQPVDLGLLEEDDEFEEFPAEDWAGLDEDEDAHVWEDNWDDDNVEDDFSNQLRAELEKHGYKMETS

SEQ ID NO: 63 - protein, 89 aa, synthetic construct
MSEKKQPVDLGLLEEDDEFEEFPAEDWAGLDEDEDAHVWEDNWDDDNVEDDFSNQLRATVLLMILVCETPYGCYVLHQKGRMCSAFLCC

SEQ ID NO: 64 - protein, 89 aa, synthetic construct; identical to SEQ ID NO: 63

SEQ ID NO: 65 - protein, 89 aa, synthetic construct
MSEKKQPVDLGLLEEDDEFEEFPAEDWAGLDEDEDAHVWEDNWDDDNVEDDFSNQLRVTVLLMILVCETLYGCYVLHQKGRMCSAFLCC

SEQ ID NO: 66 - protein, 89 aa, synthetic construct
MSEKKQPVDLGLLEEDDEFEEFPAEDWAGLDEDEDAHVWEDNWDDDNVEDDFSNQLRATILLMILVCETPYGCYVLHQKGRMCSAFLCC

SEQ ID NO: 67 - protein, 89 aa, synthetic construct
MSEKKQPVDLGLLEEDDEFEEFPAEDWAGLDEDEDAHVWEDNWDDDNVEDDFSNQLRATVLLMVLVCETPYGCYVLHQKERMCSAFLCC

SEQ ID NO: 68 - protein, 89 aa, synthetic construct
MSEKKQPVDLGLLEEDDEFEEFPAEDWAGLDEDEDAHVWEDNWDDDNVEDDFSNQLRATVLLMIKVYETPYGCYILHQKGRMCSAFLCC

SEQ ID NO: 69 - protein, 89 aa, synthetic construct; identical to SEQ ID NO: 68

SEQ ID NO: 70 - protein, 89 aa, synthetic construct; identical to SEQ ID NO: 68

SEQ ID NO: 71 - protein, 89 aa, synthetic construct
MSEKKQPVDLGLLEEDDEFEEFPAEDWAGLDEDEDAHVWEDNWDDDNVEDDFSNQLRATVLLMKKVYETPYGCYILHQKGRMCSAFLCC

SEQ ID NO: 72 - protein, 89 aa, synthetic construct; identical to SEQ ID NO: 68

SEQ ID NO: 73 - protein, 89 aa, synthetic construct; identical to SEQ ID NO: 68

SEQ ID NO: 74 - protein, 89 aa, synthetic construct; identical to SEQ ID NO: 68

SEQ ID NO: 75 - protein, 74 aa, synthetic construct
SEKKQPVDLGLLEEDDEFEEFPAEDWAGLDEDEDAHVWEDNWDDDNVEDDFSNQLRATVLLMILVAETPYGAYV

SEQ ID NO: 76 - protein, 57 aa, synthetic construct; identical to SEQ ID NO: 19

SEQ ID NO: 77 - protein, 57 aa, synthetic construct; identical to SEQ ID NO: 20

SEQ ID NO: 78 - protein, 57 aa, synthetic construct; identical to SEQ ID NO: 21

SEQ ID NO: 79 - protein, 57 aa, synthetic construct; identical to SEQ ID NO: 22

SEQ ID NO: 80 - protein, 57 aa, synthetic construct; identical to SEQ ID NO: 23

SEQ ID NO: 81 - protein, 57 aa, synthetic construct; identical to SEQ ID NO: 24

SEQ ID NO: 82 - protein, 57 aa, synthetic construct; identical to SEQ ID NO: 25

SEQ ID NO: 83 - protein, 57 aa, synthetic construct; identical to SEQ ID NO: 26

SEQ ID NO: 84 - protein, 31 aa, synthetic construct
GLAALALMVRQPAFALEIYMGVKHTALYVST

SEQ ID NO: 85 - protein, 31 aa, synthetic construct
SLLAVFPTIEAARYKMGLLYHAVAATQGMLV

SEQ ID NO: 86 - protein, 88 aa, synthetic construct
SEFEDANKEQGEVPNDDVSDELPKLLDDDEDELRGALFFQWHDWEEAEDNDWDVEDATVLLMILVAETPYGAYVLHQKGRMASAFLAA

SEQ ID NO: 87 - protein, 88 aa, synthetic construct
SELDFWDNWKDRQADLPAASDDEDFENDLAEVDHEDPLVDGNEWDGDEVEFELEEQKTVLLMILVAETPYGAYVLHQKGRMASAFLAA

SEQ ID NO: 88 - protein, 88 aa, synthetic construct
SDADEFDDDNSVWGWEDDEVEPNDAFQPLGDVRANEELDEKDHKLDEEADLQWEFELTVLLMILVAETPYGAYVLHQKGRMASAFLAA

SEQ ID NO: 89 - protein, 88 aa, synthetic construct
SQWAPLDHLESDEWGAFEDEDFNNGDVEELDPEERDDEDLFQKDVLVAKADDEEWDNTVLLMILVAETPYGAYVLHQKGRMASAFLAA

SEQ ID NO: 90 - protein, 88 aa, synthetic construct
STKKQPVDLGLLETDSEFTEFPATSWAGLSESESAHVWTSNWDSSNVTSDFSNQLRATVLLMILVAETPYGAYVLHQKGRMASAFLAA

SEQ ID NO: 91 - protein, 88 aa, synthetic construct
STKKQPVDLGLLETDDEFEEFPATSWAGLSEDESAHVWESNWDDSNVTSDFSNQLRATVLLMILVAETPYGAYVLHQKGRMASAFLAA

SEQ ID NO: 92 - protein, 88 aa, synthetic construct
STKKQPVDLGLLEEDDEFEEFPAESWAGLDEDESAHVWEDNWDDDNVTSDFSNQLRATVLLMILVAETPYGAYVLHQKGRMASAFLAA

SEQ ID NO: 93 - protein, 88 aa, synthetic construct
SEKKQPVDLGLLEEDDEFEEFPAEDWAGLDEDESAHVWEDNWDDDNVTSDFSNQLRATVLLMILVAETPYGAYVLHQKGRMASAFLAA

SEQ ID NO: 94 - protein, 88 aa, synthetic construct
SEKKQPVDLGLLEEDDEFEEFPAEDWAGLDEDEDAHVWEDNWDDDNVEDDFSNQLRAGLAALALMVRQPAFALEIYMGVKHTALYVST

SEQ ID NO: 95 - protein, 88 aa, synthetic construct
SEKKQPVDLGLLEEDDEFEEFPAEDWAGLDEDEDAHVWEDNWDDDNVEDDFSNQLRASLLAVFPTIEAARYKMGLLYHAVAATQGMLV

SEQ ID NO: 96 - protein, 58 aa, synthetic construct
MSEKKQPVDLGLLEEDDEFEEFPAEDWAGLDEDEDAHVWEDNWDDDNVTSDFSNQLRA

SEQ ID NO: 97 - protein, 25 aa, synthetic construct
TVLLMILVCETPYGCYVLHQKGRMC

SEQ ID NO: 98 - protein, 24 aa, synthetic construct
TVLLMILVCETPYGCYVLHQKGRM

SEQ ID NO: 99 - protein, 23 aa, synthetic construct
TVLLMILVCETPYGCYVLHQKGR

SEQ ID NO: 100 - protein, 22 aa, synthetic construct
TVLLMILVCETPYGCYVLHQKG

SEQ ID NO: 101 - protein, 19 aa, synthetic construct
TVLLMILVCETPYGCYVLH

SEQ ID NO: 102 - protein, 18 aa, synthetic construct
TVLLMILVCETPYGCYVL

SEQ ID NO: 103 - protein, 16 aa, synthetic construct
TVLLMILVCETPYGCY

SEQ ID NO: 104 - protein, 83 aa, synthetic construct
MSEKKQPVDLGLLEEDDEFEEFPAEDWAGLDEDEDAHVWEDNWDDDNVTSDFSNQLRATVLLMILVCETPYGCYVLHQKGRMC

SEQ ID NO: 105 - protein, 82 aa, synthetic construct
MSEKKQPVDLGLLEEDDEFEEFPAEDWAGLDEDEDAHVWEDNWDDDNVTSDFSNQLRATVLLMILVCETPYGCYVLHQKGRM

SEQ ID NO: 106 - protein, 81 aa, synthetic construct
MSEKKQPVDLGLLEEDDEFEEFPAEDWAGLDEDEDAHVWEDNWDDDNVTSDFSNQLRATVLLMILVCETPYGCYVLHQKGR

SEQ ID NO: 107 - protein, 80 aa, synthetic construct
MSEKKQPVDLGLLEEDDEFEEFPAEDWAGLDEDEDAHVWEDNWDDDNVTSDFSNQLRATVLLMILVCETPYGCYVLHQKG

SEQ ID NO: 108 - protein, 77 aa, synthetic construct
MSEKKQPVDLGLLEEDDEFEEFPAEDWAGLDEDEDAHVWEDNWDDDNVTSDFSNQLRATVLLMILVCETPYGCYVLH

SEQ ID NO: 109 - protein, 76 aa, synthetic construct
MSEKKQPVDLGLLEEDDEFEEFPAEDWAGLDEDEDAHVWEDNWDDDNVTSDFSNQLRATVLLMILVCETPYGCYVL

SEQ ID NO: 110 - protein, 74 aa, synthetic construct
MSEKKQPVDLGLLEEDDEFEEFPAEDWAGLDEDEDAHVWEDNWDDDNVTSDFSNQLRATVLLMILVCETPYGCY